SEQUENCE LISTING (30 sequences)

SEQ ID NO: 1 - DNA, 1869 bp, Artificial Sequence
  Artificially Synthesized
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagagatctggcagcggagagggcagaggaagtcttctaacatgcggtgacgtggaggagaatcccggccctagaatggccttaccagtgaccgccttgctcctgccgctggccttgctgctccacgccgccaggccgggatccggaagcggcgatagcgccacccacatcaagttcagcaagcgggacgaggacggcaaagagctggctggcgccaccatggaactgcgggacagcagcggcaagaccatcagcacctggatcagcgacggccaggtcaaagacttctacctgtaccccggcaagtacaccttcgtggaaacagccgcccctgacggctacgaggtggccacagccatcaccttcaccgtgaacgagcagggacaggtcacagtgaacggcggcggcagcggcggcagcggcggcagcggcggcagcggcggcagcggcggcagcggcggcagcgctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccatcgatagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 2 - protein, 528 aa, Artificial Sequence
  Artificially Synthesized
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKRSGSGEGRGSLLTCGDVEENPGPRMALPVTALLLPLALLLHAARPGSAHIVMVDAYKPTKASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSIDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - DNA, 1650 bp, Artificial Sequence
  Artificially Synthesized
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagagatctggcagcggagagggcagaggaagtcttctaacatgcggtgacgtggaggagaatcccggccctagaatggccttaccagtgaccgccttgctcctgccgctggccttgctgctccacgccgccaggccgggatccgcccacatcgtgatggtggacgcctacaagcccaccaagggcggcagcggcggcagcggcggcagcggcggcagcggcggcagcggcggcagcggcggcagcgctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccatcgatagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 4 - protein, 549 aa, Artificial Sequence
  Artificially Synthesized
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKRSGSGEGRGSLLTCGDVEENPGPRMALPVTALLLPLALLLHAARPGSAHIVMVDAYKPTKGGSGGSGGSGGSGGSGGSGGSASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSIDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - DNA, 1017 bp, Artificial Sequence
  Artificially Synthesized
atggccttaccagtgaccgccttgctcctgccgctggccttgctgctccacgccgccaggccgggatccggaagcggagatagcgccacccacatcaagttcagcaagcgggacgaggacggcaaagagctggctggcgccaccatggaactgcgggatagcagcggcaagaccatcagcacctggatcagcgacggccaggtcaaagacttctacctgtaccccggcaagtacaccttcgtggaaacagccgcccctgacggctatgaggtggccacagccatcaccttcaccgtgaacgagcagggacaggtcacagtgaacggcgctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccatcgatagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 6 - protein, 338 aa, Artificial Sequence
  Artificially Synthesized
MALPVTALLLPLALLLHAARPGSGSGDSATHIKFSKRDEDGKELAGATMELRDSSGKTISTWISDGQVKDFYLYPGKYTFVETAAPDGYEVATAITFTVNEQGQVTVNGASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSIDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - DNA, 1080 bp, Artificial Sequence
  Artificially Synthesized
atggccttaccagtgaccgccttgctcctgccgctggccttgctgctccacgccgccaggccgggatccggaagcggcgatagcgccacccacatcaagttcagcaagcgggacgaggacggcaaagagctggctggcgccaccatggaactgcgggacagcagcggcaagaccatcagcacctggatcagcgacggccaggtcaaagacttctacctgtaccccggcaagtacaccttcgtggaaacagccgcccctgacggctacgaggtggccacagccatcaccttcaccgtgaacgagcagggacaggtcacagtgaacggcggcggcagcggcggcagcggcggcagcggcggcagcggcggcagcggcggcagcggcggcagcgctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccatcgatagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 8 - protein, 359 aa, Artificial Sequence
  Artificially Synthesized
MALPVTALLLPLALLLHAARPGSGSGDSATHIKFSKRDEDGKELAGATMELRDSSGKTISTWISDGQVKDFYLYPGKYTFVETAAPDGYEVATAITFTVNEQGQVTVNGGGSGGSGGSGGSGGSGGSGGSASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSIDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - DNA, 798 bp, Artificial Sequence
  Artificially Synthesized
atggccttaccagtgaccgccttgctcctgccgctggccttgctgctccacgccgccaggccgggatccgcccacattgtgatggtggacgcctacaagcccaccaaggctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccatcgatagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 10 - protein, 265 aa, Artificial Sequence
  Artificially Synthesized
MALPVTALLLPLALLLHAARPGSAHIVMVDAYKPTKASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSIDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 861 bp, Artificial Sequence
  Artificially Synthesized
atggccttaccagtgaccgccttgctcctgccgctggccttgctgctccacgccgccaggccgggatccgcccacatcgtgatggtggacgcctacaagcccaccaagggcggcagcggcggcagcggcggcagcggcggcagcggcggcagcggcggcagcggcggcagcgctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccatcgatagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 12 - protein, 286 aa, Artificial Sequence
  Artificially Synthesized
MALPVTALLLPLALLLHAARPGSAHIVMVDAYKPTKGGSGGSGGSGGSGGSGGSGGSASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSIDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - DNA, 1806 bp, Artificial Sequence
  Artificially Synthesized
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagagatctggcagcggagagggcagaggaagtcttctaacatgcggtgacgtggaggagaatcccggccctagaatggccttaccagtgaccgccttgctcctgccgctggccttgctgctccacgccgccaggccgggatccggaagcggagatagcgccacccacatcaagttcagcaagcgggacgaggacggcaaagagctggctggcgccaccatggaactgcgggatagcagcggcaagaccatcagcacctggatcagcgacggccaggtcaaagacttctacctgtaccccggcaagtacaccttcgtggaaacagccgcccctgacggctatgaggtggccacagccatcaccttcaccgtgaacgagcagggacaggtcacagtgaacggcgctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccatcgatagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 14 - protein, 601 aa, Artificial Sequence
  Artificially Synthesized
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKRSGSGEGRGSLLTCGDVEENPGPRMALPVTALLLPLALLLHAARPGSGSGDSATHIKFSKRDEDGKELAGATMELRDSSGKTISTWISDGQVKDFYLYPGKYTFVETAAPDGYEVATAITFTVNEQGQVTVNGASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSIDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - DNA, 1869 bp, Artificial Sequence; identical to SEQ ID NO: 1
  Artificially Synthesized

SEQ ID NO: 16 - protein, 622 aa, Artificial Sequence
  Artificially Synthesized
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKRSGSGEGRGSLLTCGDVEENPGPRMALPVTALLLPLALLLHAARPGSGSGDSATHIKFSKRDEDGKELAGATMELRDSSGKTISTWISDGQVKDFYLYPGKYTFVETAAPDGYEVATAITFTVNEQGQVTVNGGGSGGSGGSGGSGGSGGSGGSASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSIDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - DNA, 609 bp, Artificial Sequence
  Artificially Synthesized
atggaacagaaactgattagcgaagaggatctgggttctgatctgggtaaaaaactgctggaagcagcacgtgcaggtcaggatgatgaagttcgtattctgatggcaaatggtgcagatgttaacgccaaagatttttatggtattacaccgctgcatctggcagcagcctatggtcatctggaaattgttgaagttctgctgaaacatggtgccgatgtgaatgcacatgattggaatggttggaccccgttacacctggcagcaaaatatggccatttagaaatcgtggaagtgctgttaaaacatggcgcagacgttaatgcaattgataatgcaggtaaaacccctctgcatttagcagccgcacatggccacctggaaatcgtagaggttttactgaaatacggtgcggatgtaaacgcgcaggataaatttggtaaaaccgccttcgatattagcatcgataatggtaatgaggacctggcagaaattctgcaagaggcatgcggtggtggtggtagtggtggcggtggttcagcaagcgcacatattgttatggttgatgcatataaaccgaccaaactggaacatcatcatcaccattaa

SEQ ID NO: 18 - protein, 202 aa, Artificial Sequence
  Artificially Synthesized
MEQKLISEEDLGSDLGKKLLEAARAGQDDEVRILMANGADVNAKDFYGITPLHLAAAYGHLEIVEVLLKHGADVNAHDWNGWTPLHLAAKYGHLEIVEVLLKHGADVNAIDNAGKTPLHLAAAHGHLEIVEVLLKYGADVNAQDKFGKTAFDISIDNGNEDLAEILQEACGGGGSGGGGSASAHIVMVDAYKPTKLEHHHHH

SEQ ID NO: 19 - DNA, 1164 bp, Artificial Sequence
  Artificially Synthesized
atggaacagaaactgatcagcgaagaagatctgggttctgatctgggtaaaaaactgctggaagcagcacgtgcaggtcaggatgatgaagttcgtattctgatggcaaatggtgcagatgttaatgcagatgatacctggggttggacaccgctgcatctggcagcatatcagggtcatctggaaattgttgaagtgctgctgaaaaacggtgccgatgtgaatgcctatgattatattggttggacccctctgcacttagcagcagatggccatttagaaatcgtggaagttttactgaaaaatggcgcagacgtgaacgcaagcgattatatcggtgataccccgttacacctggcagcacataatggccacctggaaatcgtagaggttctgttaaaacatggtgcggacgtgaatgcacaggataaatttggtaaaaccgccttcgatatcagcattgataatggcaatgaggatctggcagaaattctgcaagaagcatgtctcgagggtggtagtggtggctctggcggttctggtggcagtggcggtagcggtggtagtggtggcagtagaggcgtgccacacatcgtgatggtggacgcctacaagcggtacaagggcggttctggtggcagtctgccggaaaccggtggtggtagtggtggctctggcggttctggtggcagtggcggtagccaagctaaacctcaaattccgaaagataaatcaaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagccaacagatgtagaagttctagatgaacaaaaaggtaaagataaacaattaacattaattacttgtgatgattacaatgaaaagacaggcgtttgggaaaaacgtaaaatctttgtagctacagaagtcaaacatcaccaccatcatcactaa

SEQ ID NO: 20 - protein, 387 aa, Artificial Sequence
  Artificially Synthesized
MEQKLISEEDLGSDLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQEACLEGGSGGSGGSGGSGGSGGSGGSRGVPHIVMVDAYKRYKGGSGGSLPETGGGSGGSGGSGGSGGSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVEVLDEQKGKDKQLTLITCDDYNEKTGVWEKRKIFVATEVKHHHHHH

SEQ ID NO: 21 - DNA, 1164 bp, Artificial Sequence
  Artificially Synthesized
atggaacagaaactgatcagcgaagaagatctgggttctgatctgggtaaaaaactgctggaagcagcacgtgcaggtcaggatgatgaagttcgtattctgatggcaaatggtgcagatgttaacgccaaagatttttatggtattacaccgctgcatctggcagcagcctatggtcatctggaaattgttgaagttctgctgaaacatggtgccgatgtgaatgcacatgattggaatggttggaccccgttacacctggcagcaaaatatggccatttagaaatcgtggaagtgctgttaaaacatggcgcagacgttaatgcaattgataatgcaggtaaaacccctctgcatttagcagccgcacatggccacctggaaatcgtagaggttttactgaaatacggtgcggatgtaaacgcgcaggataaatttggtaaaaccgccttcgatattagcatcgataatggtaatgaggacctggcagaaattctgcaagaggcactcgagggtggtagtggtggctctggcggttctggtggcagtggcggtagcggtggtagtggtggcagtagaggcgtgccacacatcgtgatggtggacgcctacaagcggtacaagggcggttctggtggcagtctgccggaaaccggtggtggtagtggtggctctggcggttctggtggcagtggcggtagccaagctaaacctcaaattccgaaagataaatcaaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagccaacagatgtagaagttctagatgaacaaaaaggtaaagataaacaattaacattaattacttgtgatgattacaatgaaaagacaggcgtttgggaaaaacgtaaaatctttgtagctacagaagtcaaacatcaccaccatcatcactaa

SEQ ID NO: 22 - protein, 387 aa, Artificial Sequence
  Artificially Synthesized
MEQKLISEEDLGSDLGKKLLEAARAGQDDEVRILMANGADVNAKDFYGITPLHLAAAYGHLEIVEVLLKHGADVNAHDWNGWTPLHLAAKYGHLEIVEVLLKHGADVNAIDNAGKTPLHLAAAHGHLEIVEVLLKYGADVNAQDKFGKTAFDISIDNGNEDLAEILQEALEGGSGGSGGSGGSGGSGGSGGSRGVPHIVMVDAYKRYKGGSGGSLPETGGGSGGSGGSGGSGGSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVEVLDEQKGKDKQLTLITCDDYNEKTGVWEKRKIFVATEVKHHHHHH

SEQ ID NO: 23 - DNA, 825 bp, Artificial Sequence
  Artificially Synthesized
atgacctttaaactgattattaacggcaaaaccctgaaaggcgaaattaccattgaagcggtggatgcgtaggaagcggaaaaaatttttaaacagtatgcgaacgattatggcattgatggcgaatggacctatgatgatgcgaccaaaacctttaccgtgaccgaactcgagggtggtagtggtggctctggcggttctggtggcagtggcggtagcggtggtagtggtggcagtagaggcgtgccacacatcgtgatggtggacgcctacaagcggtacaagggcggttctggtggcagtctgccggaaaccggtggtggtagtggtggctctggcggttctggtggcagtggcggtagccaagctaaacctcaaattccgaaagataaatcaaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagccaacagatgtagaagttctagatgaacaaaaaggtaaagataaacaattaacattaattacttgtgatgattacaatgaaaagacaggcgtttgggaaaaacgtaaaatctttgtagctacagaagtcaaacatcaccaccatcatcactaa

SEQ ID NO: 24 - protein, 273 aa, Artificial Sequence
  Artificially Synthesized
MTFKLIINGKTLKGEITIEAVDAEAEKIFKQYANDYGIDGEWTYDDATKTFTVTELEGGSGGSGGSGGSGGSGGSGGSRGVPHIVMVDAYKRYKGGSGGSLPETGGGSGGSGGSGGSGGSQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVEVLDEQKGKDKQLTLITCDDYNEKTGVWEKRKIFVATEVKHHHHHH

SEQ ID NO: 25 - DNA, 1086 bp, Artificial Sequence
  Artificially Synthesized
atggccttaccagtgaccgccttgctcctgccgctggccttgctgctccacgccgccaggccgggatccgtgacaacactgagcggactgtctggcgagcaaggcccttctggcgatatgaccaccgaagaggatagcgccacacacatcaagttcagcaagcgcgacgaggacggcagagaacttgctggcgctaccatggaactgagagacagcagcggcaagaccatcagcacctggatctctgacggccacgtgaaggacttctatctgtaccccggcaagtacaccttcgtggaaaccgccgctcctgacggctacgaagtggccacacctatcgagttcaccgtgaacgaggatggccaagtgaccgtggatggcgaagctacagaaggcgacgcccatacagctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 26 - protein, 361 aa, Artificial Sequence
  Artificially Synthesized
MALPVTALLLPLALLLHAARPGSVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 1098 bp, Artificial Sequence
  Artificially Synthesized
atggccttaccagtgaccgccttgctcctgccgctggccttgctgctccacgccgccaggccgggatccgtgacaacactgagcggactgtctggcgagcaaggcccttctggcgatatgaccaccgaagaggatagcgccacacacatcaagttcagcaagcgcgacgaggacggcagagaacttgctggcgctaccatggaactgagagacagcagcggcaagaccatcagcacctggatctctgacggccacgtgaaggacttctatctgtaccccggcaagtacaccttcgtggaaaccgccgctcctgacggctacgaagtggccacacctatcgagttcaccgtgaacgaggatggccaagtgaccgtggatggcgaagctacagaaggcgacgcccatacagctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccatcgatagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 28 - protein, 365 aa, Artificial Sequence
  Artificially Synthesized
MALPVTALLLPLALLLHAARPGSVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSIDRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - DNA, 648 bp, Artificial Sequence
  Artificially Synthesized
atggccttaccagtgaccgccttgctcctgccgctggccttgctgctccacgccgccaggccgggatccgtgacaacactgagcggactgtctggcgagcaaggcccttctggcgatatgaccaccgaagaggatagcgccacacacatcaagttcagcaagcgcgacgaggacggcagagaacttgctggcgctaccatggaactgagagacagcagcggcaagaccatcagcacctggatctctgacggccacgtgaaggacttctatctgtaccccggcaagtacaccttcgtggaaaccgccgctcctgacggctacgaagtggccacacctatcgagttcaccgtgaacgaggatggccaagtgaccgtggatggcgaagctacagaaggcgacgcccatacagctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagctaa

SEQ ID NO: 30 - protein, 215 aa, Artificial Sequence
  Artificially Synthesized
MALPVTALLLPLALLLHAARPGSVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTASTTTPAPRPPTPAPTIASQPLSLRPEACRPAAGGAVHTRGLDFACDFWVLVVVGGVLACYSLLVTVAFIIFWVRSKRS

(19) United States
(12) Patent Application Publication
(10) Pub. No.: US 2023/0103327 A1
(43) Pub. Date: Apr. 6, 2023

(54) QUANTITATIVE CONTROL OF ACTIVITY OF ENGINEERED CELLS EXPRESSING SPYCATCHER AND SPYTAG UNIVERSAL IMMUNE RECEPTORS

(71) Applicant: The Trustees of The University of Pennsylvania, Philadelphia, PA (US)

(72) Inventors: Daniel J. Powell, Jr., Bala Cynwyd, PA (US); Andrew Tsourkas, Bryn Mawr, PA (US); Nicholas Minutolo, Philadelphia, PA (US)

(21) Appl. No.: 17/794,571

(22) PCT Filed: Jan. 23, 2021
(86) PCT No.: PCT/US2021/014811

§ 371 Date: Jul. 21, 2022

Related U.S. Application Data
(60) Provisional application No. 62/965,593, filed on Jan. 24, 2020.

(51) Int. Cl. A61K 35/17; A61P 35/00; C07K 14/725; A61K 41/00; C12N 5/10; C07K 14/705; C07K 14/47; C07K 14/315; A61K 47/68; C07K 16/32; C07K 16/28

(52) U.S. Cl. CPC A61K 35/17; A61P 35/00; C07K 14/7051; A61K 41/00; C12N 5/10; C07K 14/70578; C07K 14/47; C07K 14/315; A61K 47/6851; A61K 47/6811; C07K 16/32; C07K 16/2863; C07K 2319/02; C07K 2319/03; C07K 2319/33; A61K 38/00

(57) ABSTRACT

The invention provides methods for stimulating a universal immune receptor-mediated response in a mammal using cells engineered to express a universal immune receptor that comprises an adaptor molecule, such as a SpyCatcher or a SpyTag moiety, a transmembrane domain, and an intracellular domain for T cell activation.

### CROSS-REFERENCE TO RELATED APPLICATION

The present application is entitled to priority under 35 U.S.C. § 119(e) to U.S. Provisional Patent Application No. 62/965,593 filed Jan. 24, 2020, which is hereby incorporated by reference in its entirety herein.

### STATEMENT REGARDING FEDERALLY SPONSORED RESEARCH OR DEVELOPMENT

This invention was made with government support under CA168900 and CA016520 awarded by the National Institutes of Health. The government has certain rights in the invention.

### BACKGROUND OF THE INVENTION

Chimeric antigen receptor (CAR) T cells can mediate dramatic responses in the treatment of certain hematological malignancies leading to the FDA approval of two CD19-targeting CART cell products, tisagenlecleucel for the treatment of relapse/refractory (r/r) B cell acute lymphoblastic leukemia (B-ALL) and diffuse large B-cell lymphoma (DLBCL), and axicabtagene ciloleucel for the treatment of (r/r) large B cell lymphoma. Due to high remission rates and prolonged tumor free survival of CD19 CAR T cell treated patients, the field has expanded their use to other malignancies. Clinical trials of CAR T cells targeting other B cell specific antigens, such as BCMA, CD20, and CD22, have produced encouraging results, but several challenges, including those related to unique toxicities and subsequent relapses, need to be addressed before widespread success of CAR T cell therapy is achieved in hematologic malignancies and solid tumors.

CARs are composed of an extracellular antigen targeting domain, such as an scFv, attached to intracellular T cell signaling and costimulatory domains (e.g. 41BB and/or CD28 in tandem with CD3ζ), allowing for antigen-specific, MHC-independent T cell targeting. This design, though effective for use in single antigen targeting, presents inherent limitations to broadening the use of CAR T cells across multiple tumor types, as well as the potential for serious adverse events and toxicities.

While most drugs allow dose adjustment and follow predictable pharmacokinetics and pharmacodynamics, conventional CAR T cell therapies are living drugs that cannot be easily controlled following their infusion. Upon recognition of target antigen, the administered CAR T cells can rapidly proliferate to large numbers in the recipient and release proinflammatory cytokines, in some cases, leading to severe and sometimes fatal side effects such as cytokine release syndrome (CRS), neurotoxicity, or cerebral edema, which require medical management. In some cases, CAR T cells target and destroy non-malignant tissues that also express the targeted antigen, leading to potentially fatal on-target, off-tumor toxicity.

In addition to these challenges, the rigid CAR architecture also restricts targeting to a single tumor associated antigen (TAA). Though this approach can be effective when targeting an ubiquitous pan-B cell marker such as CD19, its effectiveness is compromised when targeting tumors with heterogeneous TAA expression or in the setting of relapsed antigen-negative tumor. About 35% of tisagenlecleucel CD19 CAR T cell recipients relapse after treatment, and more than half of relapsed disease is associated with a genetic mechanism of CD19 antigen loss due to protein truncation with a nonfunctional or absent transmembrane domain. Alternative mechanisms of antigen loss include the emergence of antigen splice variants lacking the targeted antigenic epitope, tumor cell lineage switching, trogocytosis of the CD19 antigen, and, in one rare case, accidental introduction of the CAR gene into a leukemic B cell. Single antigen targeting is also problematic in the treatment of solid tumors, which are often comprised of tumor cells with varying antigen expression patterns. In this case, selective targeting of a single antigen can cause incomplete clearance and adaptive resistance, as has been reported in the targeting of EGFRvIII.

There is a clear need in the art for compositions and methods for immune therapies for targeting tumors that minimize side effects and off-tumor toxicity. The present invention addresses this unmet need in the art.

### SUMMARY OF THE INVENTION

As described herein, the present invention relates to methods for stimulating a universal immune receptor-mediated response in a mammal using cells engineered to express a universal immune receptor that comprises an adaptor molecule, such as a SpyCatcher or a SpyTag moiety, a transmembrane domain, and an intracellular domain for T cell activation.

In one aspect, the invention provides a method for stimulating a universal immune receptor-mediated immune response to a tumor in a mammal, wherein the tumor co-expresses at least two different antigens, the method comprising (a) administering to the mammal a cell genetically modified to express an immune receptor comprising a T cell receptor intracellular signaling domain, a transmembrane domain, and an extracellular domain comprising an adaptor molecule, (b) administering to the mammal a first agent linked to a reciprocal adaptor molecule, wherein the first agent specifically binds a first antigen expressed by the tumor, and (c) administering to the mammal a second agent linked to a reciprocal adaptor molecule, wherein the second agent specifically binds a second antigen expressed by the tumor and wherein the first antigen and the second antigen are different antigens.

In another aspect, the invention provides a method of treating a cancer in a mammal in need thereof, wherein the cancer co-expresses at least two different antigens, the method comprising (a) administering to the mammal an effective amount of a cell genetically modified to express an immune receptor comprising a T cell receptor intracellular signaling domain, a transmembrane domain, and an extracellular domain comprising an adaptor molecule, (b) administering to the mammal an effective amount of a first agent linked to a reciprocal adaptor molecule, wherein the first agent specifically binds a first antigen expressed by the cancer, and (c) administering to the mammal an effective amount of a second agent linked to a reciprocal adaptor molecule, wherein the second agent specifically binds a second antigen expressed by the cancer and wherein the first antigen and the second antigen are different antigens.

In certain embodiments, the extracellular domain comprises a SpyCatcher or SpyTag.

In certain embodiments, the first and/or second agent is linked to a SpyTag or SpyCatcher.

In certain embodiments, the first and/or second agent is an antibody, an antibody fragment, a scFv, or a DARPin.

In certain embodiments, the first and/or second agent is an antibody and is human IgG.

In certain embodiments, the reciprocal adaptor molecule, SpyTag, or SpyCatcher is linked to the first and/or second agent via light activated site-specific conjugation (LASIC).

In certain embodiments, the cell is a T cell, a Natural Killer (NK) cell, a cytotoxic T lymphocyte (CTL), a macrophage, a stem cell, or a regulatory T cell.

In certain embodiments, the cell is an autologous cell.

In certain embodiments, the immune receptor further comprises an intracellular domain of a costimulatory molecule.

In certain embodiments, the costimulatory molecule is 4-1BB, CD28, CD27, CD2, CD3, OX40, CD30, CD40, PD-1, ICOS, lymphocyte function-associated antigen-1 (LFA-1), CD7, LIGHT, NKG2C, B7-H3, or a ligand that specifically binds with CD83.

In certain embodiments, the cell is contacted with the first and/or second agent prior to administration to the mammal to generate a pre-armed cell and the pre-armed cell is subsequently administered to the mammal.

In certain embodiments, the cell is administered to the mammal prior to administering the first and/or second agent to the mammal.

In another aspect, the invention provides a method of generating a level of lytic activity against a tumor, the method comprising (a) contacting an amount of cells with an amount of an agent linked to a reciprocal adaptor molecule, wherein the cells are genetically modified to express an immune receptor comprising a T cell receptor intracellular signaling domain, a transmembrane domain, and an extracellular domain wherein the extracellular domain comprises an adaptor molecule, wherein the agent specifically binds an antigen expressed by the tumor, and wherein the amount of the agent and/or the amount of cells is selected to generate the level of lytic activity against the tumor.

In another aspect, the invention provides a method of treating a cancer in a mammal in need thereof, the method comprising (a) administering to the mammal an amount of cells genetically modified to express an immune receptor comprising a T cell receptor intracellular signaling domain, a transmembrane domain, and an extracellular domain comprising an adaptor molecule, and (b) administering to the mammal an amount of an agent linked to a reciprocal adaptor molecule, wherein the agent specifically binds an antigen expressed by the cancer, wherein the amount of the cells and/or the amount of the agent is selected to provide a level of lytic activity against the cancer.

In certain embodiments, increasing the amount of agent relative to the amount of the cells increases the level of lytic activity and decreasing the amount of agent relative to the amount of the cells decreases the level of lytic activity.

In certain embodiments, the extracellular domain comprises a SpyCatcher or SpyTag.

In certain embodiments, the agent is linked to a SpyTag or SpyCatcher.

In certain embodiments, the agent is an antibody, an antibody fragment, a scFv, or a DARPin.

In certain embodiments, the agent is an antibody and is human IgG.

In certain embodiments, the reciprocal adaptor molecule, SpyTag or SpyCatcher is linked to the agent via light activated site-specific conjugation (LASIC).

In certain embodiments, the cell is a T cell, a Natural Killer (NK) cell, a cytotoxic T lymphocyte (CTL), a macrophage, a stem cell, or a regulatory T cell.

In certain embodiments, the cell is an autologous cell.

In certain embodiments, the immune receptor further comprises an intracellular domain of a costimulatory molecule.

In certain embodiments, the costimulatory molecule is 4-1BB, CD28, CD27, CD2, CD3, OX40, CD30, CD40, PD-1, ICOS, lymphocyte function-associated antigen-1 (LFA-1), CD7, LIGHT, NKG2C, B7-H3, or a ligand that specifically binds with CD83.

In certain embodiments, the cell is contacted with the agent prior to administration to the mammal to generate a pre-armed cell and the pre-armed cell is subsequently administered to the mammal.

In certain embodiments, the cell is administered to the mammal prior to administering the agent to the mammal.

In another aspect, the invention provides a method of stimulating a universal immune receptor-mediated immune response to a tumor in a mammal in need thereof, the method comprising (a) administering to the mammal a cell genetically modified to express an immune receptor comprising a T cell receptor intracellular signaling domain, a transmembrane domain, and an extracellular domain comprising an adaptor molecule, and (b) subsequently administering to the mammal an agent linked to a reciprocal adaptor molecule, wherein the agent specifically binds an antigen expressed by the tumor.

In another aspect, the invention provides a method of treating a cancer in a mammal in need thereof, the method comprising (a) administering to the mammal an effective amount of a T cell genetically modified to express an immune receptor comprising a T cell receptor intracellular signaling domain, a transmembrane domain, and an extracellular domain comprising an adaptor molecule, and (b) subsequently administering to the mammal an effective amount of an agent linked to a reciprocal adaptor molecule, wherein the agent specifically binds an antigen expressed by the cancer.

In certain embodiments, the extracellular domain comprises a SpyCatcher or SpyTag.

In certain embodiments, the agent is linked to a SpyTag or SpyCatcher.

In certain embodiments, the agent is an antibody, an antibody fragment, a scFv, or a DARPin.

In certain embodiments, the agent is an antibody and is human IgG.

In certain embodiments, the reciprocal adaptor molecule, SpyTag or SpyCatcher is linked to the agent via light activated site-specific conjugation (LASIC).

In certain embodiments, the cell is a T cell, a Natural Killer (NK) cell, a cytotoxic T lymphocyte (CTL), a macrophage, a stem cell, or a regulatory T cell.

In certain embodiments, the cell is an autologous cell.

In certain embodiments, the immune receptor further comprises an intracellular domain of a costimulatory molecule.

In certain embodiments, the costimulatory molecule is 4-1BB, CD28, CD27, CD2, CD3, OX40, CD30, CD40, PD-1, ICOS, lymphocyte function-associated antigen-1 (LFA-1), CD7, LIGHT, NKG2C, B7-H3, or a ligand that specifically binds with CD83.

In another aspect, the invention provides a method of quantifying turnover of a universal immune receptor on a cell surface, the method comprising (a) contacting a cell genetically modified to express an immune receptor comprising a T cell receptor intracellular signaling domain, a transmembrane domain, and an extracellular domain comprising an adapter molecule with an agent linked to a reciprocal adapter molecule, thereby generating an armed receptor; and (b) determining an amount of the armed receptor relative to a reference amount.

In certain embodiments, the reference amount is an amount of the armed receptor at a prior time.

In certain embodiments, the amount of the armed receptor is determined by labeling the agent and detecting the labeled agent.

In certain embodiments, the agent is labeled by linking or contacting the agent with a labeling molecule comprising a myc-tag, FLAG-tag, His-tag, HA-tag, a fluorescent protein (e.g., a green fluorescent protein (GFP)), a fluorophore (e.g., tetramethylrhodamine (TRITC)), fluorescein isothiocyanate (FITC), dinitrophenol, peridin chlorophyll protein complex, phycoerythrin (PE), histidine, biotin, streptavidin, avidin, horse radish peroxidase, palmitoylation, nitrosylation, alkalanine phosphatase, glucose oxidase, Glutathione S-transferase (GST), maltose binding protein, a radioisotope, a heavy metal, a supramagnetic nanoparticle, and any types of compounds used for radioisotope labeling including, 1,4,7,10-tetraazacyclododecane-1,4,7,10-tetraacetic acid (DOTA), diethylene triamine pentaacetic acid (DTPA), 1,4,7-triazacyclononane-1,4,7-triacetic acid (NOTA), or allophycocyanin (APC).

In certain embodiments, the method further comprises the step of (c) contacting the cell and the agent with a tumor cell.

In certain embodiments, the extracellular domain comprises a SpyCatcher.

In certain embodiments, the agent is linked to a SpyTag.

In certain embodiments, the agent is an antibody, an antibody fragment, a scFv, or a DARPin.

In certain embodiments, the agent is an antibody and is human IgG.

In certain embodiments, the cell is a T cell, a Natural Killer (NK) cell, a cytotoxic T lymphocyte (CTL), a macrophage, a stem cell, or a regulatory T cell.

In certain embodiments, the immune receptor further comprises an intracellular domain of a costimulatory molecule.

In certain embodiments, the costimulatory molecule is 4-1BB, CD28, CD27, CD2, CD3, OX40, CD30, CD40, PD-1, ICOS, lymphocyte function-associated antigen-1 (LFA-1), CD7, LIGHT, NKG2C, B7-H3, or a ligand that specifically binds with CD83.

In another aspect, the invention provides a method of stimulating a universal immune receptor-mediated immune response to a tumor in a mammal in need thereof, the method comprising (a) administering to the mammal an effective amount of a cell genetically modified to express an immune receptor comprising a T cell receptor intracellular signaling domain, an intracellular domain of 4-1BB, a transmembrane domain, and an extracellular domain comprising an adaptor molecule, and (b) administering to the mammal an effective amount of an agent linked to a reciprocal adaptor molecule, wherein the agent specifically binds an antigen expressed by the tumor, wherein the tumor is pre-determined to express the antigen at an increased level relative to a reference level.

In another aspect, the invention provides a method of treating a cancer in a mammal in need thereof, the method comprising (a) administering to the mammal an amount of a cell genetically modified to express an immune receptor comprising a T cell receptor intracellular signaling domain, an intracellular domain of 4-1BB, a transmembrane domain, and an extracellular domain comprising an adaptor molecule, and (b) administering to the mammal an amount of an agent linked to a reciprocal adaptor molecule, wherein the agent specifically binds an antigen expressed by the cancer, wherein the cancer is pre-determined to express the antigen at an increased level relative to a reference level.

In certain embodiments, the reference level is a level of expression of the antigen on healthy tissue.

In certain embodiments, the extracellular domain comprises a SpyCatcher or SpyTag.

In certain embodiments, the agent is linked to a SpyTag or SpyCatcher.

In certain embodiments, the agent is an antibody, an antibody fragment, a scFv, or a DARPin.

In certain embodiments, the agent is an antibody and is human IgG.

In certain embodiments, the SpyTag or SpyCatcher is linked to the agent via light activated site-specific conjugation (LASIC).

In certain embodiments, the cell is a T cell, a Natural Killer (NK) cell, a cytotoxic T lymphocyte (CTL), a macrophage, a stem cell, or a regulatory T cell.

In certain embodiments, the cell is an autologous cell.

In certain embodiments, the cell is contacted with agent prior to administration to the mammal to generate a pre-armed cell and the pre-armed cell is subsequently administered to the mammal.

In certain embodiments, the cell is administered to the mammal prior to administering the agent to the mammal.

In another aspect, the invention provides a genetically modified cell, a first agent, and a second agent, for use in stimulating a universal immune receptor-mediated immune response to a tumor in a mammal in need thereof, wherein the tumor co-expresses at least two different antigens, wherein the cell is genetically modified to express an immune receptor comprising a T cell receptor intracellular signaling domain, a transmembrane domain, and an extracellular domain comprising an adaptor molecule, wherein the first agent is linked to a reciprocal adaptor molecule, and wherein the first agent specifically binds a first antigen expressed by the tumor; wherein the second agent is linked to a reciprocal adaptor molecule, and wherein the second agent specifically binds a second antigen expressed by the tumor; wherein the first antigen and the second antigen are different antigens, and wherein the immune receptor, first agent, and second agent are administered to the mammal.

In another aspect, the invention provides a genetically modified cell, a first agent, and a second agent, for use in treating cancer in a mammal in need thereof, wherein the cancer co-expresses at least two different antigens, wherein the cell is genetically modified to express an immune receptor comprising a T cell receptor intracellular signaling domain, a transmembrane domain, and an extracellular domain comprising an adaptor molecule, wherein the first agent is linked to a reciprocal adaptor molecule, and wherein the first agent specifically binds a first antigen expressed by the cancer; wherein the second agent is linked to a reciprocal adaptor molecule, and wherein the second agent specifically binds a second antigen expressed by the cancer; and wherein the first antigen and the second antigen are different antigens.

In another aspect, the invention provides a genetically modified cell and an agent for use in generating a level of lytic activity against a tumor, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds a an antigen expressed by the tumor; wherein an amount of the cell is contacted with an amount of the agent and wherein the amount of the agent relative to the amount of cells is selected to generate the level of lytic activity against the tumor.

In another aspect, the invention provides a genetically modified cell and an agent for use in treating cancer in a mammal in need thereof, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds an antigen expressed by the cancer; wherein an amount of the cell and an amount of the agent is administered to the mammal, wherein the amount of the cell and/or the amount of the agent is selected to provide a level of lytic activity against the cancer.

In another aspect, the invention provides a genetically modified cell and an agent for use in stimulating a universal immune receptor-mediated immune response to a tumor in a mammal in need thereof, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds a an antigen expressed by the tumor; wherein an amount of the cell is administered to the mammal and an amount of the agent is subsequently administered to the mammal.

In another aspect, the invention provides a genetically modified cell and an agent for use in treating cancer in a mammal in need thereof, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds an antigen expressed by the cancer; wherein an amount of the cell is administered to the mammal and an amount of the agent is subsequently administered to the mammal.

In another aspect, the invention provides a genetically modified cell and an agent for use in stimulating a universal immune receptor-mediated immune response to a tumor in a mammal in need thereof, wherein the cell is genetically modified to express an immune receptor comprising a T cell receptor intracellular signaling domain, an intracellular domain of 4-1BB, a transmembrane domain, and an extracellular domain comprising an adaptor molecule, wherein the agent is linked to a reciprocal adaptor molecule, wherein the agent specifically binds a an antigen expressed by the tumor; wherein an effective amount of the cell is administered to the mammal and an effective amount of the agent is administered to the mammal, and wherein the tumor is pre-determined to express the antigen at an increased level relative to a reference level.

In another aspect, the invention provides a genetically modified cell and an agent for use in treating a cancer in a mammal in need thereof, wherein the cell is genetically modified to express an immune receptor comprising a T cell receptor intracellular signaling domain, an intracellular domain of 4-1BB, a transmembrane domain, and an extracellular domain comprising an adaptor molecule, wherein the agent is linked to a reciprocal adaptor molecule, and wherein the agent specifically binds a an antigen expressed by the tumor; wherein an effective amount of the cell is administered to the mammal and an effective amount of the agent is administered to the mammal, and wherein the tumor is pre-determined to express the antigen at an increased level relative to a reference level.

In another aspect, the invention provides use of a genetically modified cell, a first agent, and a second agent, in stimulating a universal immune receptor-mediated immune response to a tumor in a mammal in need thereof, wherein the tumor co-expresses at least two different antigens, wherein the cell is genetically modified to express an immune receptor comprising a T cell receptor intracellular signaling domain, a transmembrane domain, and an extracellular domain comprising an adaptor molecule, wherein the first agent is linked to a reciprocal adaptor molecule, and wherein the first agent specifically binds a first antigen expressed by the tumor; wherein the second agent is linked to a reciprocal adaptor molecule, and wherein the second agent specifically binds a second antigen expressed by the tumor; wherein the first antigen and the second antigen are different antigens, and wherein the immune receptor, first agent, and second agent are administered to the mammal.

In another aspect, the invention provides use of a genetically modified cell, a first agent, and a second agent, in the treatment of cancer in a mammal in need thereof, wherein the cancer co-expresses at least two different antigens, wherein the cell is genetically modified to express an immune receptor comprising a T cell receptor intracellular signaling domain, a transmembrane domain, and an extracellular domain comprising an adaptor molecule, wherein the first agent is linked to a reciprocal adaptor molecule, and wherein the first agent specifically binds a first antigen expressed by the cancer; wherein the second agent is linked to a reciprocal adaptor molecule, and wherein the second agent specifically binds a second antigen expressed by the cancer; and wherein the first antigen and the second antigen are different antigens.

In another aspect, the invention provides use of a genetically modified cell and an agent in generating a level of lytic activity against a tumor, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds a an antigen expressed by the tumor; wherein an amount of the cell is contacted with an amount of the agent and wherein the amount of the agent relative to the amount of cells is selected to generate the level of lytic activity against the tumor.

In another aspect, the invention provides use of a genetically modified cell and an agent in the treatment of cancer in a mammal in need thereof, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds an antigen expressed by the cancer; wherein an amount of the cell and an amount of the agent is administered to the mammal, wherein the amount of the cell and/or the amount of the agent is selected to provide a level of lytic activity against the cancer.

In another aspect, the invention provides use of a genetically modified cell and an agent in stimulating universal immune receptor-mediated immune response to a tumor in a mammal in need thereof, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds a an antigen expressed by the tumor; wherein an amount of the cell is administered to the mammal and an amount of the agent is subsequently administered to the mammal.

In another aspect, the invention provides use of a genetically modified cell and an in the treatment of cancer in a mammal in need thereof, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds an antigen expressed by the cancer; wherein an amount of the cell is administered to the mammal and an amount of the agent is subsequently administered to the mammal.

In another aspect, the invention provides use of a genetically modified cell and an agent in stimulating a universal immune receptor-mediated immune response to a tumor in a mammal in need thereof, wherein the cell is genetically modified to express an immune receptor comprising a T cell receptor intracellular signaling domain, an intracellular domain of 4-1BB, a transmembrane domain, and an extracellular domain comprising an adaptor molecule, wherein the agent is linked to a reciprocal adaptor molecule, and wherein the agent specifically binds a an antigen expressed by the tumor; wherein an effective amount of the cell is administered to the mammal and an effective amount of the agent is administered to the mammal, and wherein the tumor is pre-determined to express the antigen at an increased level relative to a reference level.

In another aspect, the invention provides use of a genetically modified cell and an agent in the treatment of a cancer in a mammal in need thereof, wherein the cell is genetically modified to express an immune receptor comprising a T cell receptor intracellular signaling domain, an intracellular domain of 4-1BB, a transmembrane domain, and an extracellular domain comprising an adaptor molecule, wherein the agent is linked to a reciprocal adaptor molecule, and wherein the agent specifically binds a an antigen expressed by the tumor; wherein an effective amount of the cell is administered to the mammal and an effective amount of the agent is administered to the mammal, and wherein the tumor is pre-determined to express the antigen at an increased level relative to a reference level.

### BRIEF DESCRIPTION OF THE DRAWINGS

The following detailed description of preferred embodiments of the invention will be better understood when read in conjunction with the appended drawings. For the purpose of illustrating the invention, there are examples shown in the drawings embodiments which are presently preferred. It should be understood, however, that the invention is not limited to the precise arrangements and instrumentalities of the embodiments shown in the drawings.

FIG. 1A-FIG. 1C are a series of schematic representations and images showing the development of SpyCatcher immune receptor targeting ligands. FIG. 1A: Schematic representation of Protein G-ST crosslinking to clinical-grade human IgGs. FIG. 1B: Crosslinking of Herceptin with Protein G-ST or Protein G-STDA, followed by subsequent reaction with SpyCatcher-Venus analyzed by SDS-Page gel under reducing conditions with Coomassie staining. FIG. 1C: DARPin9.26-ST and DARPin9.26-STDA reacted with SpyCatcher-Venus analyzed by SDS-Page gel with Coomassie staining.

FIG. 2A-FIG. 2E are a series of schematic representations, images, and graphs showing that the SpyCatcher Immune receptor is expressed and capable of covalent loading with SpyTag-labeled ligands. FIG. 2A: Schematic of the lentiviral SpyCatcher immune receptor constructs. FIG. 2B: SpyCatcher immune receptor expressing SKOV3 cells were incubated in culture medium containing 2000 nM RFP-ST or RFP-STDA. Covalent bond formation between the two components was detected via SDS-Page gel under reducing conditions and western blot staining for total CD3ζ protein. FIG. 2C: SpyCatcher T cells were armed with varying amounts Herceptin-ST for 1 hour at 37° C. Herceptin-ST loading on to the SpyCatcher immune receptor was detected by staining with APC polyclonal anti-human IgG and flow cytometric analysis. FIG. 2D: Comparison of covalent (DARPin9.26-ST) vs. non-covalent (DARPin9.26-ST) maximal loading of the SpyCatcher immune receptor at various concentrations. FIG. 2E: SpyCatcher T cells were incubated in wells containing various amounts of immobilized Herceptin-ST for 16 hours. Supernatant was harvested and analyzed for IFNγγ by ELISA. Error bars represent mean+/−standard deviation. Data points are averages of three replicates. Representative T cell donor is shown. *P<0.05; **P<0.01; ***P<0.001.

FIG. 3A-FIG. 3G are a series of schematic representations and graphs showing SpyCatcher T cells are capable of in vitro lytic function against a host of different antigen-expressing tumor lines. FIG. 3A: Schematic representation of armed SpyCatcher immune receptor lysis (left) and on-demand lysis (right). FIG. 3B: SpyCatcher or untransduced (Unt) T cells were armed with various concentrations of Herceptin-ST and co-cultured in the presence of SKOV3 (Her2+) tumor cells. FIG. 3C: SpyCatcher T cells were armed with antigen-specific and non-specific IgGs and co-cultured with either MDA-MB-468 (EGFR+/Her2−) or Ramos (CD20+/EGFR−) tumor cells. FIG. 3D: SpyCatcher T cells armed with antigen specific DARPins were co-culture with SKOV3, MDA-MB-468, or A1847 (EpCAM+) tumor cells expressing luciferase. FIG. 3E: DARPin9.26-ST armed SpyCatcher T cells were incubated with or without SKOV3 tumor cells (E:T=3:1) for 24 hours, removed from culture, and incubated in media+/−2000 nM DARPin9.26-ST. Receptor arming was analyzed via anti-myc staining and flow cytometric analysis. FIG. 3F: Un-armed SpyCatcher T cells were incubated with SKOV3 tumor cells for 4 hours followed by addition Herceptin-ST or Herceptin-STDA (Time=0). FIG. 3G: SpyCatcher T cells were armed with either DARPin9.26-ST or DARPin9.26-STDA at various concentrations and co-cultured with SKOV3 tumor cells expressing luciferase. FIGS. 3B and 3F: Lysis was measured using real time cell analysis. FIGS. 3C, 3D, and 3G: Residual luciferase expression was calculated after 20 hours. All co-cultures were done using a 7:1 E:T ratio. Error bars represent mean+/−standard deviation. Data points are averages of three replicates. Representative T cell donor is shown. *P<0.05; **P<0.01; ***P<0.001.

FIG. 4A-FIG. 4C are a series of schematic representations and graphs showing that simultaneous arming of SpyCatcher T cells with two targeting ligands generates a single cell product capable of dual antigen targeting. FIG. 4A: Schematic representation of single vs. dual targeting ligand loading. FIG. 4B: SpyCatcher T cells were armed with either 1000 nM myc-9.26-ST (αHer2; red), 1000 nM Flag-Eo1-ST (αEGFR; blue), or both simultaneously at 1000 nM each (green). Receptor loading was detected with a combination of fluorescently conjugated anti-myc and anti-flag antibodies and assessed via flow cytometry. FIG. 4C: Single or dual armed SpyCatcher T cells were co-cultured with Ramos cells expressing either Her2 or EGFR and luciferase. Residual luciferase expression was calculated after 20 hours. All co-cultures were done using a 7:1 E:T ratio. Data points are averages of three replicates. Representative T cell donor is shown.

FIG. 5A-FIG. 5D are a series of graphs and images showing SpyCatcher-BBζ T cells prevent tumor growth in vivo. NSG mice (n=4 per group) were injected intraperitoneally with 1×106 SKOV3 tumor cells expressing luciferase on Day 0, followed by 12.5×106 Herceptin-ST armed SpyCatcher-BBζ T cells on Day 7. Herceptin-ST was administered on day 8, followed by injections every 3 days during the dosing window (FIG. 5A. orange box; FIG. 5B. dotted line). Injection amounts indicate dose per mouse. FIG. 5A: Tumor growth was monitored by luminescence and plotted as average radiance for each individual mouse. FIG. 5B: Luminescence images of treated mice. FIG. 5C: Survival curve for mice treated in FIG. 5A. FIG. 5D: TruCount analysis of total human T cells (CD3+/CD45+) on days 7 and 21 post-T cell injection. Error bars represent mean+/−SEM (A) or mean+/−standard deviation FIG. 5D. *P<0.05; **P<0.01; ***P<0.001.

FIG. 6A-FIG. 6B are a series of images showing production of SpyTag-labeled targeting ligands. FIG. 6A: Crosslinking of Rituximab or Cetuximab with Protein G-ST, followed by subsequent reaction with SpyCatcher-Venus analyzed by SDS-Page gel under reducing conditions with Coomassie staining. FIG. 6B: DARPins Ec1-ST and E01-ST reacted with SpyCatcher-Venus analyzed by SDS-Page gel with Coomassie staining.

FIG. 7A-FIG. 7B are a series of graphs showing expression of proteins in stable cell lines via lentiviral transduction. FIG. 7A: SKOV3-GFP and SKOV3-GFP-SpyCatcher-BBζ cells were incubated in culture medium containing 2000 nM myc-RFP-ST. Cells were stained for receptor arming via anti-myc antibody and analyzed via flow cytometry. FIG. 7B: Ramos-Her2 and Ramos-EGFR cells co-stained with anti-Her2 and anti-EGFR monoclonal antibody.

FIG. 8A-FIG. 8D are a series of graphs showing the impact of antigen expression levels and dual arming on SpyCatcher T cell lytic function. FIG. 8A: SKOV3, MDA-MB-361, CRL5803, and MDA-MB-468 cell lines stained for Her2 expression. FIG. 8B: Lytic function of SpyCatcher T cells armed with 1000 nM or 100 nM Herceptin-ST against cells lines from FIG. 8A. FIG. 8C: SKOV3 cells stained for Her2 (red) and EGFR (blue) expression relative to isotype control (grey). FIG. 8D: SpyCatcher-BBζ T cells armed with either 50 nM myc-9.26-ST (αHer2; blue), 50 nM Flag-Eo1-ST (αEGFR; red), or both simultaneously at 50 nM each (green) and co-cultured with SKOV3 tumor cells. Residual luciferase expression was calculated after 20 hours. All co-cultures were done using a 7:1 E:T ratio. Statistical significance for FIG. 8D was calculated using a one-way ANOVA with Tukey post-test. Error bars represent mean+/−standard deviation. Data points are averages of three replicates. Representative T cell donor is shown. *P<0.05; **P<0.01; ***P<0.001.

FIG. 9 is a plot showing a comparison of SpyCatcher T cell and CAR T cell lytic function. SpyCatcher T cells armed with 1000 nM Herceptin-ST or 4D5 CAR T cells were co-cultured in the presence of SKOV3 (Her2+) tumor cells at various E:T ratios. Residual luciferase expression was calculated after 20 hours and normalized to untransduced T cells at each E:T ratio. Error bars represent mean+/−standard deviation. Data points are averages of three replicates. Representative T cell donor is shown.

FIG. 10 is a series of histograms showing the basal rate of armed SpyCatcher receptor turnover. Rested SpyCatcher T cells were maximally armed with Herceptin-ST and analyzed for loaded receptor every 24 hours to determine the rate of loaded receptor loss from the cell surface. Un-armed SpyCatcher T cells were stained as a negative control (grey histogram).

FIG. 11 is a plot showing that treatment with SpyCatcher-BBζ T cells did not lead to changes in weight. NSG mice (n=4 per group) were injected intraperitoneally with 1×10⁶ SKOV3 tumor cells on Day 0, followed by 12.5×10⁶Herceptin-ST armed SpyCatcher-T cells on Day 7. Herceptin-ST was administered on day 8, with subsequent injections every 3 days. Injection amounts indicate dose per mouse. (A) Weight of each mouse was monitored during course of treatment. Weight change was not observed in treatment groups, while increases in weight change occurred in control groups due to ascites formation. Error bars represent mean+/−standard deviation.

FIG. 12 is a series of plots showing SpyCatcher T cells slow the rate of tumor grown in aggressive S.C. tumor model. NSG mice (n=5 per group) were injected subcutaneously with 1×10⁶ SKOV3 tumor cells on Day 0, followed by 12.5×10⁶ SpyCatcher-T cells armed with Herceptin-ST on Day 6. 50 μg Herceptin-ST targeting ligand was administered one day after T cell infusion, followed by subsequent injections every 3 days for the entirety of the study. Tumor volume was monitored by caliper measurement (A). Black lines represent individual mice, while red lines represent the average tumor volume. Time points at which tumor growth was significantly reduced relative to SpyCatcher-BBζ alone group are noted with an asterisk (*). *P<0.05; **P<0.01; ***P<0.001.

FIG. 13 illustrates transduction efficiencies of the indicated SpyCatcher receptors which comprise GFP and a T2A site upstream of the receptor.

FIG. 14 illustrates that the SpyCatcher Immune receptor is expressed and capable of covalent loading with SpyTag-labeled ligands. Specifically, plots representing loading of SpyCatcher003-BBζ comprising GFP with myc-Her2 DARPin-SpyTag003 are shown. SpyCatcher003-BBz expressing T cells were incubated with myc-Her-2 DARPin-SpyTag003 (1000 nM) for 30 minutes. Receptor loading was detected with an Alexa647 fluorescently conjugated anti-myc antibody and assessed via flow cytometry.

FIG. 15A-FIG. 15B illustrates titration of SpyTag003 on SpyCatcher003-expressing T cells. SpyCatcher003-Δζ T cells were incubated with increasing concentrations of myc-TL-SpyTag003 for 5 or 30 min. Myc expression was measured by flow cytometry to quantify receptor loading. Peak loading is 100-200 nM and decreased at higher concentrations.

### DETAILED DESCRIPTION

The invention relates to multi-antigen targeting, controllable cell activity, and selective targeting of tumors using cells engineered to express universal immune receptors. Universal immune receptors represent a rapidly emerging form of adoptive T cell therapy with the potential to overcome safety and antigen escape challenges faced by conventional chimeric antigen receptor (CAR) T cell therapy. By decoupling antigen recognition and T cell signaling domains via bifunctional antigen-specific targeting ligands, universal immune receptors can regulate T cell effector function and target multiple antigens with a single receptor. Described herein is the development of the SpyCatcher immune receptor, the first universal immune receptor that allows for post-translational covalent attachment of targeting ligands at the T cell surface through the application of SpyCatcher-SpyTag chemistry. The SpyCatcher immune receptor redirected primary human T cells against a variety of tumor antigens via the addition of SpyTag-labeled targeting ligands, both in vitro and in vivo. SpyCatcher T cell activity relied upon the presence of both target antigen and SpyTag-labeled targeting ligand, allowing for dose-dependent control of function. Mutational disruption of covalent bond formation between the receptor and the targeting ligand still permitted redirected T cell function but significantly compromised antitumor function. Thus, the SpyCatcher immune receptor allows for rapid antigen-specific receptor assembly, multi-antigen targeting, and controllable T cell activity.

The invention relates to compositions and methods for adoptive cell therapy useful for treating a variety of disorders including cancer, infections, and autoimmune disorders. The present invention relates to a strategy for adoptive cell transfer of T cells modified to express universal immune receptors referred herein as SpyTag and SpyCatcher universal immune receptors. The receptors of the invention are molecules that combine specificity for a desired antigen with a T cell receptor-activating intracellular domain to generate a chimeric protein that exhibits a specific immune activity. In one embodiment, the SpyTag and SpyCatcher universal immune receptors of the invention comprises an extracellular label binding domain, a transmembrane domain, and a cytoplasmic domain or otherwise an intracellular domain.

The present invention provides a universal immune receptor strategy through the incorporation of short nucleotide sequences into immune receptor constructs containing intracellular signaling components for T cell activation, a transmembrane region, and a extracellular hinge region onto which a peptide tag is fused. In one embodiment, a T cell is engineered to express SpyCatcher on its surface which can be bound by any molecule that incorporates a SpyTag moiety. In another embodiment, a T cell is engineered to express SpyTag immune receptor on its surface which can be bound by any molecule that incorporates a SpyCatcher moiety. Molecules that can be bound include but are not limited to those that may redirect the T cell against a surface antigen (e.g. antibody, scFv, receptor, ligand, aptamer, etc.) or labelling agents for T cell tracking in vivo. In addition, the reciprocal tag on the redirecting molecule (e.g. antibody) may be labeled by binding, such as through conjugation, fusion, or ligation, with a labelling agent and utilized as an antigen specific labelling agent used for diagnostics, to determine patient eligibility for a clinical trial, to determine time of maximal binding to antigen in target tissue (without residual agent in healthy tissues), and as a means of monitoring response to therapy.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “auto-antigen” means, in accordance with the present invention, any self-antigen which is mistakenly recognized by the immune system as being foreign. Auto-antigens comprise, but are not limited to, cellular proteins, phosphoproteins, cellular surface proteins, cellular lipids, nucleic acids, glycoproteins, including cell surface receptors.

The term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia greata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

“Co-stimulatory ligand,” as the term is used herein, includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD3, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” tumor antigen or “overexpression” of the tumor antigen is intended to indicate an abnormal level of expression of the tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

“Parenteral” administration of an immunogenic composition includes, e.g., subcutaneous (s.c.), intravenous (i.v.), intramuscular (i.m.), or intrasternal injection, or infusion techniques.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The terms “SpyTag” and “SpyCatcher” refer to a convenient protein coupling tool that overcomes the generally weak protein-protein interaction (with “Spy” referring to the bacterium Streptococcus pyogenes). The SpyTag/SpyCatcher system is ideal for binding, labeling or immobilizing proteins as it creates irreversible peptide ligations. SpyTag is a genetically encoded peptide that forms a spontaneous amide bond upon binding its genetically encoded partner SpyCatcher. SpyTag reacts with SpyCatcher under a wide range of conditions and the after reaction the product is extremely stable (Zachari et al., 2012, PNAS vol. 109:12, pp. 690-69′7)

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

As used herein, a “universal immune receptor” is a receptor having two split but interactive parts, (i) one or more intracellular T cell signaling domains attached to an extracellular adaptor molecule and (ii) a targeting ligand that is able to bind the adaptor molecule via a reciprocal adaptor molecule and also able to specifically bind a target (e.g., an antigen on a target cell, such as an antigen expressed by a tumor cell). Thus, the targeting ligand acts as an immunologic bridge, binding both the target and the extracellular adaptor molecule on the receptor, eliciting antigen-specific T cell effector function.

As used herein, an “adaptor molecule” and a “reciprocal adaptor molecule” (or “tag” and “reciprocal tag”) refer to a pair of components in a binding pair system, where the each of the pair specifically binds to the other. Examples of binding pair systems include, but are not limited to, Spycatcher/SpyTag and biotin/avidin. The binding may be covalent or non-covalent binding. The “adaptor molecule” refers to one of the pair, and the “reciprocal adaptor molecule” refers to the binding partner of the adaptor molecule. Thus, in a Spycatcher/SpyTag system, the adaptor molecule for example, may be SpyCatcher and the reciprocal adaptor molecule would be SpyTag, or vice-versa, where the adaptor molecule may be SpyTag and the reciprocal adaptor molecule would be SpyCatcher.

As used herein, the term “armed receptor” or “armed universal immune receptor” is meant to refer to a universal immune receptor where its two interactive parts are linked or bound to each other, i.e., the part containing the intracellular T cell signaling domain(s) and extracellular adaptor molecule is linked or bound to the targeting ligand capable of binding to the adaptor molecule via the reciprocal adaptor molecule and capable of specifically binding to a target (e.g., an antigen expressed by a cell, such as a tumor cell), thus rendering the “armed receptor” or “armed universal immune receptor” capable of binding the target and eliciting antigen-specific T cell effector function. In contrast, an “unarmed receptor” or “unarmed universal immune receptor” refers to a universal immune receptor where its two interactive parts are not linked or bound to each other, i.e., the part containing the intracellular T cell signaling domain(s) and extracellular adaptor molecule is not linked or bound to the targeting ligand capable of binding to the adaptor molecule via the reciprocal adaptor molecule and capable of specifically binding to a target (e.g., an antigen expressed by a cell, such as a tumor cell).

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

Current gene-engineered cellular therapy is restricted in antigen specificity, patient accessibility, and tumor or cell type. An alternative to creating a CAR, which has a fixed antigen specificity, is to create a universal T cell receptor that allows the specificity of the T cell receptor to be changed on demand. This is achieved by engineering T cells that express a unique peptide or protein tag, which can be labeled post-translationally with a complementary binding partner that is fused or attached to a targeting ligand (e.g. antibody, antibody binding domain, protein scaffold, aptamer, receptor, etc.), imaging agent, hapten, enzyme, etc. The benefits of creating a single engineered universal T cell that can subsequently be labeled with any targeting ligand includes, but is not limited to, the ability to create large panels of T cells very rapidly and easily, prepare individual T cells with multiple specificities, and change T cell specificity over time.

In order to expand the prototypic CAR architecture to allow for temporal and quantitative control of T cell effector function, a tag-specific receptor capitalizing on the interaction between biotin and avidin was created and disclosed in Urbanska et al., Cancer Research 2012, 72 (7), 1844-1852. This receptor, called the biotin-binding immune receptor, belongs to a rapidly expanding class of orthogonal receptors termed universal immune receptors (UIRs) (Minutolo et al., Frontiers in Oncology 2019, 9, 176; 25-35; Clemenceau et al., Blood 2006, 107 (12), 4669-4677. Tamada et al., lin Cancer Res 2012, 18 (23), 6436-6445; Kudo et al., Cancer Research 2013, 74 (1); Urbanska et al., Journal of Translational Medicine 2014, 12 (1), 347; Ochi et al., Cancer Immunology 2014, 2 (3), 249-262; Feldmann et al., Oncotarget 2014, 5 (0); Kim et al., J Am Chem Soc 2015, 137 (8), 2832-2835; Rodgers et al., Proc. Natl. Acad. Sci. U.S.A. 2016, 113 (4); Ma et al., Proc. Natl. Acad. Sci. U.S.A. 2016, 113 (4), E450-E458; Cho et al., Cell 2018, No. Mol. Ther. Oncolytics 3 2016). UIRs are comprised of two split but interactive parts, (i) standard intracellular T cell signaling domains attached to an extracellular adaptor protein and (ii) targeting ligands that are able to bind the adaptor protein. The targeting ligand acts as an immunologic bridge, binding both the TAA on target cells and the extracellular adaptor on the receptor, eliciting antigen-specific T cell effector function.

By decoupling TAA recognition from T cell signaling, UIRs can address several issues currently faced by CAR T cells. UIRs rely on the presence of antigen specific targeting ligands to drive T cell effector function, therefore allowing dose-dependent control of T cell effector function (Minutolo et al., Frontiers in Oncology 2019, 9, 176), and mitigation of toxicities seen with CAR T cells due to rapid activation and expansion, such as CRS (Rodgers et al., Proc. Natl. Acad. Sci. U.S.A. 2016, 113 (4)). Additionally, UIRs provide modular platforms capable of targeting multiple TAAs with a single cell product via the use of multiple targeting ligands, addressing issues of TAA heterogeneity and relapse associated with single antigen-loss.

At present, all UIRs rely on noncovalent interactions between the targeting ligand and the UIR. Here, we detail the development of a next generation UIR system that allows for post-translational covalent attachment of targeting domains to the receptor via the use of SpyCatcher-SpyTag chemistry. Developed by Zakeri et al., interaction between the protein SpyCatcher and its cognate peptide SpyTag leads to rapid and spontaneous formation of a covalent amide bond (Zakeri et al., Proc. Natl. Acad. Sci. U.S.A. 2012, 109 (12), E690-7). Reaction between the peptide-protein pair occurs across a range of physiologically relevant temperatures and pH levels, making it a potential candidate for use in in vivo (Zakeri et al., Proc. Natl. Acad. Sci. U.S.A. 2012, 109 (12), E690-7).

SpyCatcher-SpyTag has also been used to label membrane resident receptors in live mammalian cell culture, as well as in transgenic C. elegans, endogenously expressing proteins containing SpyCatcher and SpyTag domains (Bedbrook et al., Chem. Biol. 2015, 22 (8), 1108-1121). Taken together, the broad range of robust reactivity conditions coupled with its use in both mammalian cells and live organisms makes the SpyCatcher-SpyTag system a favorable choice for the de novo assembly of UIR architectures that mimic CARs.

Described herein is a SpyCatcher immune receptor that was developed that contains the SpyCatcher protein as its extracellular domain attached to standard second-generation CAR intracellular signaling domains. Addition of TAA-specific targeting ligands labeled with SpyTag allows for on-demand formation of a CAR-like receptor through spontaneous, autocatalytic isopeptide bond formation. Primary human T cells expressing the SpyCatcher immune receptor can be quantitatively loaded with targeting ligands, allowing for dose-titratable control of redirected T cell effector function and tumor cell lysis. Even as a single cell product, SpyCatcher immune receptor T cells can recognize an array of tumor antigens via addition of clinical-grade antibodies site-specifically labeled with SpyTag, as well as targeting ligands to which SpyTag is genetically fused. Results described herein demonstrate flexibility and efficacy of the SpyCatcher immune receptor system.

### Multiple Antigen Targeting

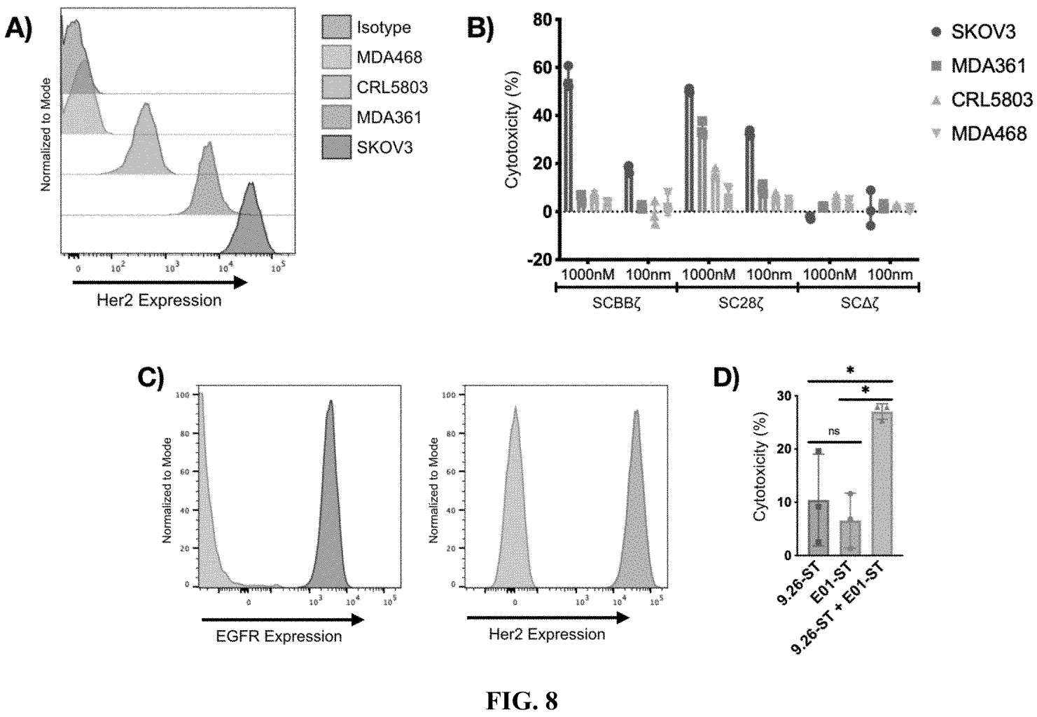

The present invention relates to use of cells engineered to express universal immune receptors to targeting multiple antigens, in particular two or more distinct antigens expressed by a tumor. As further described herein, it was found that dual armed SpyCatcher-BBζ T achieved increased tumor lysis relative to single armed cells alone (FIG. 8D). It was demonstrated that arming SpyCatcher T cells with two antibodies of distinct specificity (at a specified dose) enhanced activity against cancer cells co-expressing two distinct antigens.

Thus, in one aspect, a method is provided for stimulating a universal immune receptor-mediated immune response to a tumor in a mammal, wherein the tumor co-expresses at least two different antigens. In another aspect, a method of treating a cancer in a mammal in need thereof is provided, wherein the cancer co-expresses at least two different antigens. In one embodiment, the method include the steps of (a) administering to the mammal a cell genetically modified to express an immune receptor comprising a T cell receptor intracellular signaling domain, a transmembrane domain, and an extracellular domain comprising a adaptor molecule, (b) administering to the mammal a first agent linked to a SpyCatcher or SpyTag, wherein the first agent specifically binds a first antigen expressed by the tumor, and (c) administering to the mammal a second agent linked to a reciprocal adaptor molecule, wherein the second agent specifically binds a second antigen expressed by the tumor and wherein the first antigen and the second antigen are different antigens.

In some aspects, a genetically modified cell, a first agent, and a second agent, are provided for use in stimulating a universal immune receptor-mediated immune response to a tumor in a mammal in need thereof or for use in treating cancer in a mammal in need thereof, wherein the tumor or cancer co-expresses at least two different antigens, wherein the cell is genetically modified to express an immune receptor comprising a T cell receptor intracellular signaling domain, a transmembrane domain, and an extracellular domain comprising an adaptor molecule, wherein the first agent is linked to a reciprocal adaptor molecule, and wherein the first agent specifically binds a first antigen expressed by the tumor or cancer; wherein the second agent is linked to a reciprocal adaptor molecule, and wherein the second agent specifically binds a second antigen expressed by the tumor or cancer; wherein the first antigen and the second antigen are different antigens, and wherein the immune receptor, first agent, and second agent are administered to the mammal.

In other aspects, use of a genetically modified cell, a first agent, and a second agent, in stimulating a universal immune receptor-mediated immune response to a tumor in a mammal in need thereof or in treating a cancer in a mammal in need thereof is provided, wherein the tumor or cancer co-expresses at least two different antigens, wherein the cell is genetically modified to express an immune receptor comprising a T cell receptor intracellular signaling domain, a transmembrane domain, and an extracellular domain comprising an adaptor molecule, wherein the first agent is linked to a reciprocal adaptor molecule, and wherein the first agent specifically binds a first antigen expressed by the tumor or cancer; wherein the second agent is linked to a reciprocal adaptor molecule, and wherein the second agent specifically binds a second antigen expressed by the tumor or cancer; wherein the first antigen and the second antigen are different antigens, and wherein the immune receptor, first agent, and second agent are administered to the mammal.

In some embodiments, the adaptor molecule is SpyCatcher and reciprocal adaptor molecule is SpyTag. In some embodiments, the adaptor molecule is SpyTag and reciprocal adaptor molecule is SpyCatcher.

In some embodiments, the administration of both the first and second agent results in enhanced anti-tumor activity compared to administration of only the first agent or only the second agent. In some embodiments, a similar or the same level of anti-tumor activity is achieved when administering a lower amount of the first agent and/or second agent in combination with each other compared to the level of anti-tumor activity achieved when only the first agent or only the second agent is administered.

In some embodiments, the adaptor molecule is SpyCatcher and reciprocal adaptor molecule is SpyTag. In some embodiments, the adaptor molecule is SpyTag and reciprocal adaptor molecule is SpyCatcher.

In some embodiments, the extracellular domain comprises a SpyCatcher or SpyTag. In one embodiment, the extracellular domain comprises SpyCatcher.

In some embodiments, the agent is linked to a SpyTag or SpyCatcher. In one embodiment, the agent is linked to SpyTag. In another embodiment, the agent is an antibody, an antibody fragment, a scFv, or a DARPin. In another embodiment, the agent is an antibody. In one embodiment, the agent is human IgG.

In one embodiment, the SpyTag or SpyCatcher is linked to the agent via light activated site-specific conjugation (LASIC). In one embodiment, the cell is a T cell, a Natural Killer (NK) cell, a cytotoxic T lymphocyte (CTL), a macrophage, a stem cell, or a regulatory T cell. In one embodiment, the cell is a T cell. In another embodiment, the cell is an autologous cell.

In another embodiment, the universal immune receptor further comprises an intracellular domain of a costimulatory molecule. In some embodiments, the costimulatory molecule is 4-1BB, CD28, CD27, CD2, CD3, OX40, CD30, CD40, PD-1, ICOS, lymphocyte function-associated antigen-1 (LFA-1), CD7, LIGHT, NKG2C, B7-H3, or a ligand that specifically binds with CD83. In one embodiment, the antigen is HER2, EGFR, EpCAM, or CD20.

In one embodiment, the cell is contacted with the agent prior to administration to the mammal to generate a pre-armed cell and the pre-armed cell is subsequently administered to the mammal. In another embodiment, wherein the cell is administered to the mammal prior to administering the agent to the mammal. In some embodiments, the amount of agent administered and/or the number or amount is selected or adjusted to generate a desired level of lytic activity.

### Control of Lytic Activity

In another aspect, a method for generating a level of lytic activity against a tumor is provided. In yet another aspect, a method of treating cancer in a subject (e.g., a mammal) is provided, where the method provides for control of the level of lytic activity against the cancer. While most drugs allow dose adjustment and follow predictable pharmacokinetics and pharmacodynamics, conventional CAR T cell therapies are living drugs that cannot be easily controlled following their infusion. Upon recognition of target antigen, the administered CAR T cells can rapidly proliferate to large numbers in the recipient and release proinflammatory cytokines, in some cases, leading to severe and sometimes fatal side effects. As described elsewhere herein, it was demonstrated that primary human T cells expressing the SpyCatcher immune receptor can be quantitatively loaded with targeting ligands, allowing for dose-titratable control of redirected T cell effector function and tumor cell lysis.

In one embodiment, the method includes the step of contacting an amount of cells with an amount of an agent linked to a reciprocal adaptor molecule, wherein the cells are genetically modified to express an immune receptor comprising a T cell receptor intracellular signaling domain, a transmembrane domain, and an extracellular domain wherein the extracellular domain comprises an adaptor molecule, wherein the agent specifically binds an antigen expressed by the tumor, and wherein the amount of the agent and/or the number of cells is selected to generate the level of lytic activity against the tumor. In another embodiment, the method includes the steps of (a) administering to the mammal an amount of cells genetically modified to express an immune receptor comprising a T cell receptor intracellular signaling domain, a transmembrane domain, and an extracellular domain comprising an adaptor molecule, (b) administering to the mammal an amount of an agent linked to a reciprocal adaptor molecule, wherein the agent specifically binds an antigen expressed by the cancer, wherein the amount of the cells and/or the amount of the agent is selected to provide a level of lytic activity against the cancer.

In one aspect, a genetically modified cell and an agent for use in generating a level of lytic activity against a tumor or for use in a treatment of cancer in a mammal in need thereof, are provided, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds a an antigen expressed by the tumor or cancer; wherein an amount of the cell is contacted with an amount of the agent and wherein the amount of the agent relative to the amount of the cell is selected to generate the level of lytic activity against the tumor or cancer.

In another aspect, use of a genetically modified cell and an agent in generating a level of lytic activity against a tumor or in a treatment of cancer in a mammal in need thereof, is provided, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds a an antigen expressed by the tumor or cancer; wherein an amount of the cell is contacted with an amount of the agent and wherein the amount of the agent relative to the amount of the cell is selected to generate the level of lytic activity against the tumor or cancer.

The level of lytic activity can be any level selected, for example, a level of lytic activity that effectively lyses tumor cells and minimizes undesirable side effects in the mammal. The level of lytic activity can be a pre-determined level of lytic activity. In some embodiments, the level of lytic activity is at least 10%, at least 20%, at least 30%, at least 40%, at least 50%, at least 60%, at least 70%, at least 80%, or at least 90% cytoxicity. In one embodiment, increasing the amount of agent relative to the amount of the cells (e.g., increasing arming dose) increases the level of lytic activity. In another embodiment, decreasing the amount of agent relative to the amount of the cells (e.g., decreasing arming dose) decreases the level of lytic activity.

In an embodiment, the method comprises determining the tumor load or cancer load of the mammal and selecting the amount of the agent relative to the amount of the cell to generate the level of lytic activity against the tumor or cancer based on the tumor load or cancer load. The tumor load or cancer load can be determined by standard techniques such measuring the size of the tumor(s) for a solid tumor, the number of circulating cancer cells (for instance for a leukemia patient), or determining the levels of a cancer marker.

In an embodiment, the method comprises administering a dose of the cells and/or the amount of the agent to the mammal, determining if the treatment is being effective and if not increasing the dose of the cells and/or the amount of the agent. In an embodiment, the mammal is administered with more of the agent. In an alternate embodiment, the mammal is administered with a second (or further) agent as defined herein.

In some embodiments, the adaptor molecule is SpyCatcher and reciprocal adaptor molecule is SpyTag. In some embodiments, the adaptor molecule is SpyTag and reciprocal adaptor molecule is SpyCatcher.

In some embodiments, the extracellular domain comprises a SpyCatcher or SpyTag. In one embodiment, the extracellular domain comprises SpyCatcher.

In some embodiments, the agent is linked to a SpyTag or SpyCatcher. In one embodiment, the agent is linked to SpyTag. In another embodiment, the agent is an antibody, an antibody fragment, a scFv, or a DARPin. In another embodiment, the agent is an antibody. In one embodiment, the agent is human IgG.

In one embodiment, the adaptor molecule or reciprocal adaptor molecule is linked to the agent via light activated site-specific conjugation (LASIC). In one embodiment, the SpyTag or SpyCatcher is linked to the agent via light activated site-specific conjugation (LASIC). In one embodiment, the cell is a T cell, a Natural Killer (NK) cell, a cytotoxic T lymphocyte (CTL), a macrophage, a stem cell, or a regulatory T cell. In one embodiment, the cell is a T cell. In another embodiment, the cell is an autologous cell.

In another embodiment, the universal immune receptor further comprises an intracellular domain of a costimulatory molecule. In some embodiments, the costimulatory molecule is 4-1BB, CD28, CD27, CD2, CD3, OX40, CD30, CD40, PD-1, ICOS, lymphocyte function-associated antigen-1 (LFA-1), CD7, LIGHT, NKG2C, B7-H3, or a ligand that specifically binds with CD83.

In one embodiment, the antigen is HER2, EGFR, EpCAM, or CD20.

In one embodiment, the cell is contacted with the agent prior to administration to the mammal to generate a pre-armed cell and the pre-armed cell is subsequently administered to the mammal. In another embodiment, wherein the cell is administered to the mammal prior to administering the agent to the mammal. In some embodiments, the amount of agent administered and/or the number or amount of cells administered is selected or adjusted to generate a desired level of lytic activity.

### “On-Demand” Targeting

In another aspect, the present invention provides methods of stimulating a universal immune receptor-mediated immune response to a tumor in a mammal and methods of treating a cancer in a mammal in need thereof which use “on-demand” targeting of the tumor. As described elsewhere herein, it was found that armed, but not unarmed, SpyCatcher T cells were capable of targeting and killing antigen expressing tumor cells, and it was further demonstrated that unarmed SpyCatcher T cell effector function could be temporally triggered upon later addition of targeting ligand to un-armed SpyCatcher T cells. Thus, unarmed SpyCatcher T cells can be administered to a subject, where the unarmed cells are inert in the presence of cancer cells in the subject, and a targeting ligand can be administered at a later time to arm the SpyCatcher T cells and trigger tumor lysis, allowing cancer killing capacity to be triggered “on-demand.”

Thus, in one embodiment, the method includes the step of (a) administering to the mammal a cell genetically modified to express an immune receptor comprising a T cell receptor intracellular signaling domain, a transmembrane domain, and an extracellular domain comprising an adaptor molecule, (b) subsequently administering to the mammal an agent linked to a reciprocal adaptor molecule, wherein the agent specifically binds an antigen expressed by the tumor.

In another aspect, a genetically modified cell and an agent for use in stimulating a universal immune receptor-mediated immune response to a tumor in a mammal in need thereof or for use in treating cancer in a mammal in need thereof, are provided, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds a an antigen expressed by the tumor; wherein an amount of the cell is administered to the mammal and an amount of the agent is subsequently administered to the mammal.

In another aspect, use of a genetically modified cell and an agent in stimulating a universal immune receptor-mediated immune response to a tumor in a mammal in need thereof or in treating cancer in a mammal in need thereof, is provided, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds a an antigen expressed by the tumor; wherein an amount of the cell is administered to the mammal and an amount of the agent is subsequently administered to the mammal.

In some embodiments, the adaptor molecule is SpyCatcher and reciprocal adaptor molecule is SpyTag. In some embodiments, the adaptor molecule is SpyTag and reciprocal adaptor molecule is SpyCatcher.

In some embodiments, the extracellular domain comprises a SpyCatcher or SpyTag. In one embodiment, the extracellular domain comprises SpyCatcher.

In some embodiments, the agent is linked to a SpyTag or SpyCatcher. In one embodiment, the agent is linked to SpyTag. In another embodiment, the agent is an antibody, an antibody fragment, a scFv, or a DARPin. In another embodiment, the agent is an antibody. In one embodiment, the agent is human IgG.

In one embodiment, the adaptor molecule or reciprocal adaptor molecule is linked to the agent via light activated site-specific conjugation (LASIC). In one embodiment, the SpyTag or SpyCatcher is linked to the agent via light activated site-specific conjugation (LASIC). In one embodiment, the cell is a T cell, a Natural Killer (NK) cell, a cytotoxic T lymphocyte (CTL), a macrophage, a stem cell, or a regulatory T cell. In one embodiment, the cell is a T cell. In another embodiment, the cell is an autologous cell.

In another embodiment, the universal immune receptor further comprises an intracellular domain of a costimulatory molecule. In some embodiments, the costimulatory molecule is 4-1BB, CD28, CD27, CD2, CD3, OX40, CD30, CD40, PD-1, ICOS, lymphocyte function-associated antigen-1 (LFA-1), CD7, LIGHT, NKG2C, B7-H3, or a ligand that specifically binds with CD83.

In one embodiment, the antigen is HER2, EGFR, EpCAM, or CD20.

In some embodiments, the amount of agent administered and/or the number or amount of cells administered is selected or adjusted to generate a desired level of lytic activity.

Targeting Tumor based on Antigen Expression Level

It was surprisingly found that SpyCatcher-BB ζ T cells, but not SpyCatcher-28ζ T cells, armed with high concentration antibody were sensitive to high antigen expressing target cells but not low antigen expressing target cells. The findings have important implications of safety of the T cells when applied with antibodies targeting high risk antigens expressed on healthy tissue.

Thus, in other aspects, a method of stimulating a universal immune receptor-mediated immune response to a tumor in a mammal in need thereof and a method of treating cancer in a mammal in need thereof are provided, using cells engineered to express immune receptors containing an intracellular domain of 4-1BB. In one embodiment, the method includes the steps of (a) administering to the mammal an amount of a cell genetically modified to express a universal immune receptor comprising a T cell receptor intracellular signaling domain, an intracellular domain of 4-1BB, a transmembrane domain, and an extracellular domain comprising an adaptor molecule, and (b) administering to the mammal an amount of an agent linked to a reciprocal adaptor molecule, wherein the agent specifically binds an antigen expressed by the cancer, wherein the cancer is pre-determined to express the antigen at an increased level relative to a reference level. Methods for determining the level of antigen expression in a tumor can be any well-known in the art, e.g., immunohistochemical methods.

In some aspects, a genetically modified cell and an agent for use in stimulating a universal immune receptor-mediated immune response to a tumor in a mammal in need thereof or for use in treating a cancer in a mammal in need thereof, are provided, wherein the cell is genetically modified to express an immune receptor comprising a T cell receptor intracellular signaling domain, an intracellular domain of 4-1BB, a transmembrane domain, and an extracellular domain comprising an adaptor molecule, wherein the agent is linked to a reciprocal adaptor molecule, and wherein the agent specifically binds a an antigen expressed by the tumor or cancer; wherein an effective amount of the cell is administered to the mammal and an effective amount of the agent is administered to the mammal, and wherein the cancer or tumor is pre-determined to express the antigen at an increased level relative to a reference level.

In other aspects, use of a genetically modified cell and an agent in stimulating a universal immune receptor-mediated immune response to a tumor in a mammal in need thereof or in treating a cancer in a mammal in need thereof, is provided, wherein the cell is genetically modified to express an immune receptor comprising a T cell receptor intracellular signaling domain, an intracellular domain of 4-1BB, a transmembrane domain, and an extracellular domain comprising an adaptor molecule, wherein the agent is linked to a reciprocal adaptor molecule, and wherein the agent specifically binds a an antigen expressed by the tumor or cancer; wherein an effective amount of the cell is administered to the mammal and an effective amount of the agent is administered to the mammal, and wherein the tumor or cancer is pre-determined to express the antigen at an increased level relative to a reference level.

In some embodiments, the adaptor molecule is SpyCatcher and reciprocal adaptor molecule is SpyTag. In some embodiments, the adaptor molecule is SpyTag and reciprocal adaptor molecule is SpyCatcher.

In some embodiments, the extracellular domain comprises a SpyCatcher or SpyTag. In one embodiment, the extracellular domain comprises SpyCatcher.

In some embodiments, the agent is linked to a SpyTag or SpyCatcher. In one embodiment, the agent is linked to SpyTag. In another embodiment, the agent is an antibody, an antibody fragment, a scFv, or a DARPin. In another embodiment, the agent is an antibody. In one embodiment, the agent is human IgG.

In one embodiment, the adaptor molecule or reciprocal adaptor molecule is linked to the agent via light activated site-specific conjugation (LASIC). In one embodiment, the SpyTag or SpyCatcher is linked to the agent via light activated site-specific conjugation (LASIC). In one embodiment, the cell is a T cell, a Natural Killer (NK) cell, a cytotoxic T lymphocyte (CTL), a macrophage, a stem cell, or a regulatory T cell. In one embodiment, the cell is a T cell. In another embodiment, the cell is an autologous cell.

In one embodiment, the antigen is HER2, EGFR, EpCAM, or CD20.

In one embodiment, the cell is contacted with the agent prior to administration to the mammal to generate a pre-armed cell and the pre-armed cell is subsequently administered to the mammal. In another embodiment, wherein the cell is administered to the mammal prior to administering the agent to the mammal. In some embodiments, the amount of agent administered and/or the number or amount of cells administered is selected or adjusted to generate a desired level of lytic activity.

### Compositions

The present invention provides a type of universal immune receptor comprising an extracellular and intracellular domain. The extracellular domain comprises a unique binding element otherwise referred to as an extracellular binding domain. In some embodiments, the extracellular domain also comprises a hinge region. The intracellular domain or otherwise the cytoplasmic domain comprises a costimulatory signaling region and a zeta chain portion. The costimulatory signaling region refers to a portion of the universal immune receptor comprising the intracellular domain of a costimulatory molecule. Costimulatory molecules are cell surface molecules other than antigens receptors or their ligands that are required for an efficient response of lymphocytes to antigen.

Between the extracellular domain and the transmembrane domain of the universal immune receptor, or between the cytoplasmic domain and the transmembrane domain of the universal immune receptor, there may be incorporated a spacer domain. As used herein, the term “spacer domain” generally means any oligo- or polypeptide that functions to link the transmembrane domain to, either the extracellular domain or, the cytoplasmic domain in the polypeptide chain. A spacer domain may comprise up to 300 amino acids, preferably 10 to 100 amino acids and most preferably 25 to 50 amino acids.

The present invention includes retroviral and lentiviral vector constructs capable of expressing a universal immune receptor that can be directly transduced into a cell. The present invention also includes an RNA or DNA construct that can be directly transfected into a cell. A method for generating mRNA for use in transfection involves in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the gene to be expressed, and a polyA tail, typically 50-2000 bases in length. RNA so produced can efficiently transfect different kinds of cells. In one embodiment, the template includes sequences for the universal immune receptor.

Polynucleotide vectors can be prepared that encode the universal immune receptor. Cell lines can then be engineered to express the universal immune receptor, and cells expressing the universal immune receptor can be isolated and used in the methods disclosed herein.

The cells expressing the universal immune receptor may be formulated for administration to a subject using techniques known to the skilled artisan. Formulations of the cells expressing the universal immune receptor may include pharmaceutically acceptable excipient(s). Excipients included in the formulations will have different purposes depending, for example, on the nature of the label, the cell composition, and the mode of administration. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, and lubricating agents.

Preferably, the universal immune receptor comprises an extracellular domain, a transmembrane domain and a cytoplasmic domain. The extracellular domain and transmembrane domain can be derived from any desired source of such domains.

Extracellular Label Binding Domain

The extracellular domain of the universal immune receptor of the present invention comprises an adaptor molecule (e.g., SpyTag or SpyCatcher). In one embodiment, SpyTag or SpyCatcher is associated with its reciprocal tag, i.e. SpyCatcher or SpyTag, respectively, which can be bound to any molecule of interest. In one embodiment, the extracellular domain or reciprocal tag may consist of an Ig heavy chain which may in turn be covalently associated with Ig light chain by virtue of the presence of CH1 and hinge regions, or may become covalently associated with other Ig heavy/light chain complexes by virtue of the presence of hinge, CH2 and CH3 domains. In the latter case, the heavy/light chain complex that becomes joined to the chimeric construct may constitute an antibody with a specificity distinct from the antibody specificity of the chimeric construct. Depending on the function of the antibody, the desired structure and the signal transduction, the entire chain may be used or a truncated chain may be used, where all or a part of the CH1, CH2, or CH3 domains may be removed or all or part of the hinge region may be removed.

The present invention is based upon a universal strategy of adoptive T cell therapy using the SpyCatcher or SpyTag system that binds any molecule that comprises the reciprocal tag: SpyTag or SpyCatcher, respectively. Any molecule capable of being bound, such as fused, conjugated, ligated or labeled, with a SpyTag or a SpyCatcher moiety is encompassed in the present invention. For example, the molecule of the present invention encompasses a protein (an antibody, antibody fragment, scFv, protein scaffold, a receptor, a ligand), peptide, oligonucleotides, an imaging/labeling agent and the like. Examples of other types of labels useful in the present invention include myc-tag, FLAG-tag, His-tag, HA-tag, fluorescent protein (e.g. green fluorescent protein (GFP)), fluorophore (e.g. Tetramethylrhodamine (TRITC), fluorescein isothiocyanate (FITC)), dinitrophenol, peridinin chlorophyll protein complex, green fluorescent protein, biotin, phycoerythrin (PE), histidine, streptavidin, avidin, horse radish peroxidase, palmitoylation, nitrosylation, alkalanine phosphatase, glucose oxidase, Glutathione S-transferase (GST), maltose binding protein, fluorescein and any types of fluorescent materials including quantum dot nanocrystals, radioisotopes, and any types of compounds used for radioisotope labeling including, 1,4,7,10-tetraazacyclododecane-1,4,7,10-tetraacetic acid (DOTA), diethylene triamine pentaacetic acid (DTPA), and 1,4,7-triazacyclononane-1,4,7-triacetic acid (NOTA).

In one embodiment, the universal immune receptor of the invention comprises a target-specific binding element otherwise referred to as an antigen binding domain. The choice of moiety depends upon the type and number of ligands that define the surface of a target cell. For example, the antigen binding domain may be chosen to recognize a ligand that acts as a cell surface marker on target cells associated with a particular disease state. Thus examples of cell surface markers that may act as ligands for the antigen moiety domain in the universal immune receptor of the invention include those associated with viral, bacterial and parasitic infections, autoimmune disease and cancer cells.

In one embodiment, a retroviral or lentiviral vector comprises a universal immune receptor designed to express a SpyTag or SpyCatcher on the T cell surface, which can be bound to any molecule that incorporates a SpyTag or a SpyCatcher moiety, respectively. In one embodiment, the molecule comprises a target- or antigen-specific binding element. The binding domain may be chosen to recognize a target, ligand, or antigen that acts as a cell surface marker on target cells associated with a particular disease or disorder. In some embodiments, the antigen can be associated with specific hyperproliferative disorders. In certain aspects, the hyperproliferative disorder antigens of the present invention are derived from cancers including, but not limited to, primary or metastatic melanoma, mesothelioma, thymoma, lymphoma, sarcoma, lung cancer, liver cancer, non-Hodgkin's lymphoma, Hodgkin's lymphoma, leukemias, uterine cancer, cervical cancer, bladder cancer, kidney cancer and adenocarcinomas such as breast cancer, prostate cancer, ovarian cancer, pancreatic cancer, and the like. In general, the cancer may be any type of cancer as long as the cancerous tumor has a cell surface antigen that may be recognized by the present SpyTag/SpyCatcher universal immune receptor system.

The present invention is not limited to universal immune receptors directed to tumor antigens. Rather any target, ligand, or antigen associated with a disease or disorder may be targeted by the universal immune receptor of the invention. For example, in one embodiment, the universal immune receptor of the invention is targeted to a viral antigen. In another embodiment, the universal immune receptor of the invention is targeted to a self-antigen. Self-antigens are antigens normally tolerated by a healthy subject, but induce an adaptive immune response in autoimmune disorders. For example, epidermal cadherin is a self-antigen that induces an autoimmune response in pemphigus vulgaris. Other non-limiting self-antigens (listed with their associated autoimmune disorder) which are useful to be targeted by the composition of the invention, include pancreatic β-cell antigen (insulin-dependent diabetes mellitus), acetylcholine receptor (Myasthenia gravis), thyroid-stimulating hormone receptor (Graves' disease), insulin receptor (hypoglycemia), glycoprotein (immune thrombocytopenic purpura), Rh blood group antigens (autoimmune hemolytic anemia), rheumatoid factor IgG complexes (rheumatoid arthritis), and myelin basic protein (experimental autoimmune encephalomyelitis, multiple sclerosis).

Transmembrane Domain

With respect to the transmembrane domain, the SpyTag/SpyCatcher universal immune receptor of the present invention can be designed to comprise a transmembrane domain that is fused to the extracellular domain that is bound to the SpyTag/SpyCatcher system. In one embodiment, the transmembrane domain that naturally is associated with another the domain in the universal immune receptor is used. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154.

Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the universal immune receptor. A glycine-serine doublet provides a particularly suitable linker.

Cytoplasmic Domain

The cytoplasmic domain or otherwise the intracellular signaling domain of the SpyTag/SpyCatcher universal immune receptor of the invention is responsible for activation of at least one of the normal effector functions of the immune cell in which the universal immune receptor has been placed in. The term “effector function” refers to a specialized function of a cell. Effector function of a T cell, for example, may be cytolytic activity or helper activity including the secretion of cytokines. Thus the term “intracellular signaling domain” refers to the portion of a protein that transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Preferred examples of intracellular signaling domains for use in the universal immune receptor of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM containing primary cytoplasmic signaling sequences that are of particular use in the invention include those derived from TCR zeta, FcR gamma, FcR beta, CD3 gamma, CD3 delta, CD3 epsilon, CD5, CD22, CD79a, CD79b, and CD66d. It is particularly preferred that cytoplasmic signaling molecule in the universal immune receptor of the invention comprises a cytoplasmic signaling sequence derived from CD3 zeta.

In a preferred embodiment, the cytoplasmic domain of the universal immune receptor can be designed to comprise the CD3-zeta signaling domain by itself or combined with any other desired cytoplasmic domain(s) useful in the context of the universal immune receptor of the invention. For example, the cytoplasmic domain of the universal immune receptor can comprise a CD3 zeta chain portion and a costimulatory signaling region. The costimulatory signaling region refers to a portion of the universal immune receptor comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD3, CD7, LIGHT, NKG2C, B7-H3, and a ligand that specifically binds with CD83, and the like. Thus, while the invention is exemplified primarily with CD28 and 4-1BB as the co-stimulatory signaling element, other costimulatory elements are within the scope of the invention.

The cytoplasmic signaling sequences within the cytoplasmic signaling portion of the universal immune receptor of the invention may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the cytoplasmic domain is designed to comprise the signaling domain of CD3-zeta. In another embodiment, the cytoplasmic domain is designed to comprise any combination of CD3-zeta, CD28, 4-1BB, and the like.

In one embodiment, the SpyTag/SpyCatcher universal immune receptor may substantially lack a cytoplasmic domain or the cytoplasmic domain may substantially lack signaling capacity. In such an embodiment, the SpyTag/SpyCatcher universal immune receptor may serve as an anchoring molecule to attach tagged proteins, including but not limited to antibodies, cytokines and imaging moieties, to the T cell surface.

### Labeling of Molecules

The present invention encompasses a SpyTag/SpyCatcher universal immune receptor directed to a labeled molecule with a reciprocal SpyTag/SpyCatcher moiety. The molecule of interest can be labeled by any method known in the art. For example, in one embodiment, a composition comprising the labeled molecule is bound to the universal immune receptor. The molecule may be, for example, any molecule comprising an antigen binding domain or fragment thereof, such as an antibody, an antibody fragment, and a scFv, a peptide, a protein scaffold, a nucleic acid, aptamer, ribozyme, small molecule, and the like. In some aspects, the molecule of interest lacks a label but still interacts with the universal immune receptor. For example, in one embodiment, a composition comprising the molecule of interest lacks a label, while a second composition comprising the universal immune receptor contains the label.

In one aspect, a method of generating a universal immune receptor targeting ligand is provided, wherein the method comprises linking adaptor molecule or reciprocal adaptor molecule to a targeting ligand via light activated site-specific conjugation (LASIC) of the adaptor molecule or reciprocal adaptor molecule to the targeting ligand. In another aspect, compositions comprising a targeting ligand are provided, wherein the targeting ligand linked to an adaptor molecule or reciprocal adaptor via light activated site-specific conjugation (LASIC). In some embodiments, the adaptor molecule is SpyCatcher or SpyTag. In some embodiments, the reciprocal adaptor molecule is SpyCatcher or SpyTag. In some other embodiments, the targeting ligand is an antibody, an antibody fragment, a scFv, or a DARPin. In some embodiments, the targeting ligand is human IgG. In some embodiments, the targeting ligand is a clinical-grade antibody.

Any known set of molecules may be used to target the SpyTag/SpyCatcher universal immune receptor to an antigen of interest. Non-limiting examples of molecules include any molecule comprising an antigen binding domain or fragment thereof, such as an antibody, an antibody fragment, and a scFv, a peptide, a protein scaffold, an oligonucleotide, a small molecule, and a ligand. Well-known examples of labels that can be bound, such as fused, conjugated, or ligated, or attached to the molecule include myc-tag, FLAG-tag, His-tag, HA-tag, fluorescent protein (e.g. green fluorescent protein (GFP)), a fluorophore (e.g. tetramethylrhodamine (TRITC), fluorescein isothiocyanate (FITC)), dinitrophenol, peridinin chlorophyll protein complex, green fluorescent protein, biotin, phycoerythrin (PE), histidine, streptavidin, avidin, horse radish peroxidase, palmitoylation, nitrosylation, alkalanine phosphatase, glucose oxidase, Glutathione S-transferase (GST), maltose binding protein, any types of fluorescent materials including quantum dot nanocrystals, radioisotopes, and any types of compounds used for radioisotope labeling including, 1,4,7,10-tetraazacyclododecane-1,4,7,10-tetraacetic acid (DOTA), diethylene triamine pentaacetic acid (DTPA), and 1,4,7-triazacyclononane-1,4,7-triacetic acid (NOTA).

Labeling of the molecules with any of such labels may be performed directly or indirectly. The label may be conjugated to the molecule using techniques such as chemical coupling and chemical cross-linkers. Alternatively, polynucleotide vectors can be prepared that encode the labeled molecules as fusion proteins. Cell lines can then be engineered to express the labeled molecule, and the labeled molecule can be isolated from culture media, purified and used in the methods disclosed herein. A labeled amino acid, labeled peptide, labeled protein, or molecular reporter may also be ligated to the molecule via expressed protein ligation (e.g. using sortase, inteins, etc.).

The labeled molecule may be formulated for administration to a subject using techniques known to the skilled artisan. Formulations of the labeled molecule may include pharmaceutically acceptable excipient(s). Excipients included in the formulations will have different purposes depending, for example, on the nature of the label, the antigen binding composition, and the mode of administration. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, and lubricating agents.

In another embodiment, the universal immune receptor comprises an extracellular domain that binds to an unlabeled intermediate, which in turn binds the molecule or labeled molecule.

### Vectors

The present invention encompasses a DNA construct comprising the sequence of a SpyTag/SpyCatcher universal immune receptor, wherein the sequence comprises the nucleic acid sequence of an extracellular domain operably linked to the nucleic acid sequence of an intracellular domain. In one embodiment, the extracellular domain comprises a SpyTag domain. In another embodiment, the extracellular domain comprises a SpyCatcher domain. An exemplary intracellular domain that can be used in the universal immune receptor of the invention includes but is not limited to the intracellular domain of CD3-zeta, CD28, 4-1BB, and the like. In some instances, the universal immune receptor can comprise any combination of CD3-zeta, CD28, 4-1BB, and the like.

In one embodiment, the SpyCatcher/SpyTag Immune Receptor construct comprises SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30. Table 1 below summarizes the sequence identifiers of the SpyCatcher/SpyTag Immune Receptor constructs:

TABLE 1
Sequence identifiers for the present SpyCatcher/SpyTag constructs

| SEQ ID NO: | IDENTITY |
|---|---|
| SEQ ID NO: 1 | pELNS(SpeI)-GFP-SpyTag-CD28-Zeta, nucleic acid sequence |
| SEQ ID NO: 2 | pELNS(SpeI)-GFP-SpyTag-CD28-Zeta, amino acid sequence |
| SEQ ID NO: 3 | pELNS(SpeI)-GFP-SpyTag-GGS-CD28-Zeta, nucleic acid sequence |
| SEQ ID NO: 4 | pELNS(SpeI)-GFP-SpyTag-GGS-CD28-Zeta, amino acid sequence |
| SEQ ID NO: 5 | pELNS(SpeI)-SpyCatcher-CD28-Zeta, nucleic acid sequence |
| SEQ ID NO: 6 | pELNS(SpeI)-SpyCatcher-CD28-Zeta, amino acid sequence |
| SEQ ID NO: 7 | pELNS(SpeI)-SpyCatcher-GGS-CD28-Zeta, nucleic acid sequence |
| SEQ ID NO: 8 | pELNS(SpeI)-SpyCatcher-GGS-CD28-Zeta, amino acid sequence |
| SEQ ID NO: 9 | pELNS(SpeI)-SpyTag-CD28-Zeta, nucleic acid sequence |
| SEQ ID NO: 10 | pELNS(SpeI)-SpyTag-CD28-Zeta, amino acid sequence |
| SEQ ID NO: 11 | pELNS(SpeI)-SpyTag-GGS-CD28-Zeta, nucleic acid sequence |
| SEQ ID NO: 12 | pELNS(SpeI)-SpyTag-GGS-CD28-Zeta, amino acid sequence |
| SEQ ID NO: 13 | pELNS(SpeI)-GFP-SpyCatcher-CD28-Zeta, nucleic acid sequence |
| SEQ ID NO: 14 | pELNS(SpeI)-GFP-SpyCatcher-CD28-Zeta amino acid sequence |
| SEQ ID NO: 15 | pELNS(SpeI)-GFP-SpyCatcher-GGS-CD28-Zeta, nucleic acid sequence |
| SEQ ID NO: 16 | pELNS(SpeI)-GFP-SpyCatcher-GGS-CD28-Zeta, amino acid sequence |
| SEQ ID NO: 17 | Myc tag-DARPin9.26-SpyTag- His Tag, nucleic acid sequence |
| SEQ ID NO: 18 | Myc tag-DARPin9.26-SpyTag- His Tag, amino acid sequence |
| SEQ ID NO: 19 | SpyTag003 EGFR DARPIN, nucleic acid sequence |
| SEQ ID NO: 20 | SpyTag003 EGFR DARPIN, amino acid sequence |
| SEQ ID NO: 21 | SpyTag003 HER2 DARPIN, nucleic acid sequence |
| SEQ ID NO: 22 | SpyTag003 HER2 DARPIN, amino acid sequence |
| SEQ ID NO: 23 | SpyTag003 Protein G, nucleic acid sequence |
| SEQ ID NO: 24 | SpyTag003 Protein G, amino acid sequence |
| SEQ ID NO: 25 | SpyCatcher003 BBz, nucleic acid sequence |
| SEQ ID NO: 26 | SpyCatcher003 BBz, amino acid sequence |
| SEQ ID NO: 27 | SpyCatcher003 28z, nucleic acid sequence |
| SEQ ID NO: 28 | SpyCatcher003 28z, amino acid sequence |
| SEQ ID NO: 29 | SpyCatcher003 delta zeta, nucleic acid sequence |
| SEQ ID NO: 30 | SpyCatcher003 delta zeta, amino acid sequence |

In one embodiment, the universal immune receptor of the invention comprises a SpyCatcher or SpyTag domain, a human CD8 alpha hinge and transmembrane domain, and a CD28 and CD3-zeta signaling domains.

The nucleic acid sequences coding for the desired molecules can be obtained using recombinant methods known in the art, using standard techniques. Alternatively, the gene of interest can be produced synthetically.

The present invention also provides vectors in which a DNA of the present invention is inserted (e.g. DNA encoding a SpyTag or a SpyCatcher domain). Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In brief summary, the expression of natural or synthetic nucleic acids encoding universal immune receptors is typically achieved by operably linking a nucleic acid encoding the universal immune receptor (e.g. SpyTag/SpyCatcher) polypeptide or portions thereof to a promoter, and incorporating the construct into an expression vector. The vectors can be suitable for replication and integration in eukaryotes. Typical cloning vectors contain transcription and translation terminators, initiation sequences, and promoters useful for regulation of the expression of the desired nucleic acid sequence.

The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a universal immune receptor polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.);

cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### RNA Transfection

In one embodiment, the genetically modified T cells of the invention are modified through the introduction of RNA. In one embodiment, an in vitro transcribed SpyTag or SpyCatcher universal immune receptor RNA can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is the universal immune receptor of the present invention. For example, the template for the RNA of the universal immune receptor comprises an extracellular domain comprising a label binding domain; a transmembrane domain comprising the hinge and transmembrane domain of CD8a; and a cytoplasmic domain comprises the signaling domain of CD3-zeta.

In one embodiment, the DNA to be used for PCR contains an open reading frame. The DNA can be from a naturally occurring DNA sequence from the genome of an organism. In one embodiment, the DNA is a full length gene of interest of a portion of a gene. The gene can include some or all of the 5′ and/or 3′ untranslated regions (UTRs). The gene can include exons and introns. In one embodiment, the DNA to be used for PCR is a human gene. In another embodiment, the DNA to be used for PCR is a human gene including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA that is used for transfection. Methods for performing PCR are well known in the art. Primers for use in PCR are designed to have regions that are substantially complementary to regions of the DNA to be used as a template for the PCR. “Substantially complementary”, as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one preferred embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In a preferred embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

### Genetically Modified T Cells

In some embodiments, the SpyTag or SpyCatcher universal immune receptor sequences are delivered into cells using a retroviral or lentiviral vector. Universal immune receptor-expressing retroviral and lentiviral vectors can be delivered into different types of eukaryotic cells as well as into tissues and whole organisms using transduced cells as carriers or cell-free local or systemic delivery of encapsulated, bound or naked vectors. The method used can be for any purpose where stable expression is required or sufficient.

In other embodiments, the SpyTag or SpyCatcher universal immune receptor sequences are delivered into cells using in vitro transcribed mRNA. In vitro transcribed mRNA universal immune receptor can be delivered into different types of eukaryotic cells as well as into tissues and whole organisms using transfected cells as carriers or cell-free local or systemic delivery of encapsulated, bound or naked mRNA. The method used can be for any purpose where transient expression is required or sufficient.

The disclosed methods can be applied to the modulation of T cell activity in basic research and therapy, in the fields of cancer, stem cells, acute and chronic infections, and autoimmune diseases, including the assessment of the ability of the genetically modified T cell to kill a target cancer cell. In some embodiments, the modified T cells are labeled and can be traced in vivo once transferred to a subject.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the universal immune receptor mRNAs with different structures and combination of their domains. For example, varying of different intracellular effector/costimulator domains on multiple universal immune receptors in the same cell allows determination of the structure of the receptor combinations which assess the highest level of cytotoxicity against multi-antigenic targets—after the universal immune receptors are associated with a reciprocal tag that is attached to an antigen-binding element—and at the same time lowest cytotoxicity toward normal cells.

One advantage of RNA transfection methods of the invention is that RNA transfection is essentially transient and a vector-free: An RNA transgene can be delivered to a lymphocyte and expressed therein following a brief in vitro cell activation, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population.

Genetic modification of T cells with in vitro-transcribed RNA (IVT-RNA) makes use of two different strategies both of which have been successively tested in various animal models. Cells are transfected with in vitro-transcribed RNA by means of lipofection or electroporation. Preferably, it is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

RNA has several advantages over more traditional plasmid or viral approaches. Gene expression from an RNA source does not require transcription and the protein product is produced rapidly after the transfection. Further, since the RNA has to only gain access to the cytoplasm, rather than the nucleus, and therefore typical transfection methods result in an extremely high rate of transfection. In addition, plasmid based approaches require that the promoter driving the expression of the gene of interest be active in the cells under study.

In another aspect, the RNA construct can be delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

### Sources of T Cells

Prior to expansion and genetic modification of the T cells of the invention, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as Ficoll™ separation. In one preferred embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL′ gradient or by counterflow centrifugal elutriation. A specific subpopulation of T cells, such as CD3⁺, CD28⁺, CD4⁺, CD8⁺, CD45RA⁺, and CD45RO⁺T cells, can be further isolated by positive or negative selection techniques. For example, in one embodiment, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4⁺, CD25⁺, CD62L^{hi}, GITR⁺, and FoxP3⁺. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4⁺ T cells express higher levels of CD28 and are more efficiently captured than CD8⁺ T cells in dilute concentrations. In one embodiment, the concentration of cells used is 5×10⁶/ml. In other embodiments, the concentration used can be from about 1×10⁵/ml to 1×10⁶/ml, and any integer value in between.

In other embodiments, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In a further embodiment of the present invention, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

Whether prior to or after genetic modification of the T cells to express a desirable universal immune receptor, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention are expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one embodiment, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4⁺ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular embodiment an increase of from about 1 to about 3 fold is observed as compared to the expansion observed using a ratio of 1:1. In one embodiment, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1. In one particular embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In one preferred embodiment, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one embodiment, a ratio of particles to cells of 1:1 or less is used. In one particular embodiment, a preferred particle: cell ratio is 1:5. In further embodiments, the ratio of particles to cells can be varied depending on the day of stimulation. For example, in one embodiment, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular embodiment, the ratio of particles to cells is 1:1 on the first day of stimulation and adjusted to 1:5 on the third and fifth days of stimulation. In another embodiment, particles are added on a daily or every other day basis to a final ratio of 1:1 on the first day, and 1:5 on the third and fifth days of stimulation. In another embodiment, the ratio of particles to cells is 2:1 on the first day of stimulation and adjusted to 1:10 on the third and fifth days of stimulation. In another embodiment, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment the cells (for example, 10⁴ to 10⁹ T cells) and beads (for example, DYNABEADS® M-450 CD3/CD28 T paramagnetic beads at a ratio of 1:1) are combined in a buffer, preferably PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In one embodiment of the present invention, the mixture may be cultured for several hours (about 3 hours) to about 14 days or any hourly integer value in between. In another embodiment, the mixture may be cultured for 21 days. In one embodiment of the invention the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

T cells that have been exposed to varied stimulation times may exhibit different characteristics. For example, typical blood or apheresed peripheral blood mononuclear cell products have a helper T cell population (T_{H}, CD4⁺) that is greater than the cytotoxic or suppressor T cell population (T_{C}, CD8⁺). Ex vivo expansion of T cells by stimulating CD3 and CD28 receptors produces a population of T cells that prior to about days 8-9 consists predominately of T_{H} cells, while after about days 8-9, the population of T cells comprises an increasingly greater population of T_{C} cells. Accordingly, depending on the purpose of treatment, infusing a subject with a T cell population comprising predominately of T_{H} cells may be advantageous. Similarly, if an antigen-specific subset of T_{C} cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

### Therapeutic Application

The present invention encompasses a cell (e.g., T cell) modified to express a universal immune receptor that combines a SpyTag or SpyCatcher domain with an intracellular domain of a T cell receptor. In some instances, the universal immune receptor further comprises an intracellular domain of one or more co-stimulatory molecule. Therefore, in some instances, the modified T cell can elicit a universal immune receptor-mediated T-cell response.

The invention provides the use of a universal immune receptor to redirect the specificity of a primary T cell to any given molecule that incorporates a SpyTag or SpyCatcher moiety. Thus, the present invention also provides a method for stimulating a T cell-mediated immune response to a target cell population or tissue in a mammal comprising the steps of tagging/labeling the target antigen (with a SpyTag or SpyCatcher moiety for instance) and administering to the mammal a T cell that expresses a universal immune receptor, wherein the universal immune receptor comprises a binding moiety that specifically interacts with the tagged/labeled target, an intracellular domain of a TCR (e.g., intracellular domain of human CD3zeta), and a costimulatory signaling region.

In one aspect, the invention relates to use of cells engineered to express universal immune receptors to targeting multiple antigens, in particular two or more distinct antigens expressed by a tumor. As further described herein, it was found that dual armed SpyCatcher-BBζ T achieved increased tumor lysis relative to single armed cells alone (FIG. 8D). It was demonstrated that arming SpyCatcher T cells with two antibodies of distinct specificity (at a specified dose) enhanced activity against cancer cells co-expressing two distinct antigens.

In another aspect, a method for generating a level of lytic activity against a tumor is provided. In yet another aspect, a method of treating cancer in a subject (e.g., a mammal) is provided, where the method provides control of the level of lytic activity against the cancer. As described elsewhere herein, it was demonstrated that primary human T cells expressing the SpyCatcher immune receptor can be quantitatively loaded with targeting ligands, allowing for dose-titratable control of redirected T cell effector function and tumor cell lysis. Compared to conventional CAR T cell therapies, where upon recognition of target antigen, the administered CAR T cells can rapidly proliferate to large numbers in the recipient and release proinflammatory cytokines, controllable T cell activity and lysis in a patient could help minimize undesirable side effects stemming from uncontrolled rapid proliferation of the administered T cells.

In yet another aspect, methods of stimulating a universal immune receptor-mediated immune response to a tumor in a mammal and methods of treating a cancer in a mammal in need thereof which use “on-demand” targeting of the tumor, are provided. Unarmed SpyCatcher T cells can be administered to a subject, where the unarmed cells are inert in the presence of cancer cells in the subject, and a targeting ligand can be administered at a later time to arm the SpyCatcher T cells and trigger tumor lysis, allowing cancer killing capacity to be triggered “on-demand.”

In still other aspects, a method of stimulating a universal immune receptor-mediated immune response to a tumor in a mammal in need thereof and a method of treating cancer in a mammal in need thereof, are provided, using cells engineered to express universal immune receptors containing an intracellular domain of 4-1BB. It was surprisingly found that SpyCatcher-BB ζ T cells, but not SpyCatcher-28ζ T cells, armed with high concentration antibody were sensitive to high antigen expressing target cells but not low antigen expressing target cells. The findings have important implications of safety of the T cells when applied with antibodies targeting high risk antigens expressed on healthy tissue.

In one embodiment, tagging/labeling of the target molecule comprises administering to the mammal a composition which comprises a tagged and/or a labeled molecule. Administration of the T cell and the tagged/labeled molecule may occur in any order. For example, in one embodiment, the labeled molecule is administered to the mammal prior to administration of the T cell. In another embodiment, the T cell is administered to the mammal prior to administration of the tagged/labeled antigen. In another embodiment, the universal immune receptor may be bound to tagged/labeled molecule prior to administration of the T cell to the mammal.

The tagged/labeled molecule or compositions comprising the tagged/labeled molecule may be administered to a subject using modes and techniques known to the skilled artisan. Exemplary modes include, but are not limited to subcutaneously, intradermally, intratumorally, intranodally, intramedullary, intramuscularly, by intravenous (i.v.) injection, intra-arterial, intracardiac, intra-articular, intrasynovial, intracranial, intraspinal, intrathecal or intraperitoneally. In one embodiment, the labeled compositions are administered to a patient by intradermal or subcutaneous injection. In another embodiment, the labeled compositions of the present invention are preferably administered by i.v. injection. The tagged/labeled molecule or compositions comprising the tagged/labeled molecule may be injected directly into a tumor, lymph node, or site of infection. The tagged/labeled molecule or compositions comprising the tagged/labeled molecule are administered in an amount which is effective for tagging/labeling the target antigen and is effective for treating the patient. The particular amount administered to the subject will vary between wide limits, depending upon the location, source, identity, extent and severity of the disorder, the age and condition of the individual to be treated, etc.

In one embodiment, the present invention includes a type of cellular therapy where T cells are genetically modified to express a SpyTag or SpyCatcher universal immune receptor. The SpyTag or SpyCatcher is associated with its reciprocal tag: SpyCatcher or SpyTag, which is bound, such as fused, labeled, ligated or conjugated, to a molecule comprising an antigen-binding domain. The universal immune receptor system is then administered to a recipient in need thereof. In another embodiment, the SpyTag- or SpyCatcher-bound molecule is administered to the mammal prior to the genetically modified cell. In yet another embodiment, the universal immune receptor is bound to the SpyTag- or the SpyCatcher-bound molecule prior to administering the genetically modified cell to the mammal. The administered cell is able to kill tumor cells in the recipient.

While disclosed herein specifically are lentiviral vectors encoding a SpyCatcher domain, along with human CD8a hinge and transmembrane domain, and human CD28 and CD3zeta signaling domains, the invention should be construed to include any number of variations for each of the components of the construct as described elsewhere herein.

The present invention also provides a method of simultaneously targeting multiple targets, such as two or more distinct targets. For example, in one embodiment, multiple distinct molecules are tagged/labeled, either directly or indirectly with SpyCatcher/SpyTag. For example, in one embodiment, distinct molecules are each bound, such as labeled, conjugated, ligated or fused, with a SpyCatcher or a SpyTag, specific for each molecule, and subsequently, the multiple distinct molecules bound to a SpyCatcher or a SpyTag are administered to the mammal. Administration of a genetically modified T cell expressing a universal immune receptor comprising the reciprocal tag (SpyTag/SpyCatcher) allows for the targeting of the modified T cells to each of the multiple distinct reciprocally tagged molecules. In one embodiment, the method comprises administering multiple distinct SpyTag- or SpyCatcher-bound molecules to the mammal prior to administering the genetically modified cell to the mammal. In such an embodiment, the universal immune receptor may bind the multiple distinct SpyTag- or SpyCatcher-bound molecules. In another embodiment, the method comprises binding the universal immune receptor with the multiple distinct SpyTag- or SpyCatcher-bound molecules prior to administering the genetically modified cell to the mammal.

In another embodiment, multiple distinct molecules are targeted by multi-specific T cells. For example, genetically modified T cells expressing a SpyCatcher/SpyTag universal immune receptor are bound with multiple distinct molecules that are bound, such as labeled, conjugated, ligated or fused, with the reciprocal SpyCatcher/SpyTag, specific for each of the multiple distinct molecules. These multi-specific T cells are then administered to the mammal. Administration of genetically modified multi-specific T cells allows for the targeting of the modified T cells to each of the multiple distinct reciprocally tagged molecules.

The present invention also provides a method of sequential targeting of multiple distinct targets, such as two or more distinct targets (e.g., two or more distinct antigens). For example, in one embodiment, a first molecule is tagged/labeled, either directly or indirectly. For example, in one embodiment, a first SpyTag or SpyCatcher tagged molecule, specific for a first antigen, is administered to the mammal. In one embodiment, the method comprises administering a genetically modified T cell expressing a universal immune receptor comprising a complementary SpyTag or SpyCatcher binding domain binds the first tagged molecule, thereby targeting the T cell to the first antigen. In one embodiment, the method comprises tagging/labeling a second molecule, either directly or indirectly. For example, in one embodiment, a second tagged molecule (such as a second SpyTag or SpyCatcher-tagged molecule), specific for a second antigen, is administered to the mammal. Genetically modified T cells expressing the universal immune receptor comprising a SpyTag or SpyCatcher domain bind the second tagged molecule and is thus directed to the second antigen. In one embodiment, the method comprises sequentially administering multiple distinct SpyTag- or SpyCatcher-bound molecules to the mammal prior to administering the genetically modified cell to the mammal. In another embodiment, the method comprises allowing sufficient time to elapse between administration of the first and second tagged molecules, to allow for clearance of cells expressing the first antigen prior to directing the T cell to the second antigen. In yet another embodiment, the method comprises sequentially binding the universal immune receptor with multiple distinct SpyTag- or SpyCatcher-bound molecules prior to administering the genetically modified cell to the mammal. As would be understood by those skilled in the art, the method of the invention encompasses further iterations for the targeting of additional distinct target antigens.

In one embodiment, the present invention provides a method of using a universal immune receptor to target a tagged antigen for treating cancer. Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the SpyTag or SpyCatcher universal immune receptors of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases). In one embodiment, the present invention provides a method of using the universal immune receptor to target an antigen associated with a virus, bacteria, parasite, or other infection in order to treat the infection.

In one embodiment, the present invention provides a method of using the SpyTag or SpyCatcher universal immune receptor to target a self-antigen to treat an autoimmune disorder. In one embodiment, the method comprises genetically modifying an immunosuppressive T regulatory cell to express a universal immune receptor comprising a SpyTag or SpyCatcher domain. In one embodiment comprises tagging a molecule comprising a self-antigen binding domain with a reciprocal tag (SpyCatcher or SpyTag, respectively) and administering a T regulatory cell modified to express a universal immune receptor comprising a SpyTag or SpyCatcher domain. In one embodiment, targeting of the T regulatory cell to the self-antigen reduces the autoimmune response directed to the self-antigen. For example, in one embodiment, activation of the genetically modified T regulatory cell via binding to the targeted self-antigen reduces cytolytic T cell proliferation. Non-limiting examples of autoimmune disorders treatable by way of the present invention includes multiple sclerosis, inflammatory bowel disease, Crohn's disease, ulcerative colitis, graft-versus-host disease, rheumatoid arthritis, psoriasis, dermatitis, autoimmune type I diabetes, systemic lupus erythematosus, Hashimoto's thyroiditis, myasthenia gravis, and the like. As it would be understood by the skilled artisan, the present invention is useful for treating any autoimmune disorder characterized by an autoimmune response against a self-antigen. The present invention encompasses treatment of autoimmune disorders where the self-antigen is currently known, and where the self-antigen is elucidated in the future.

However, the invention should not be construed to be limited to solely to the antigen targets and diseases disclosed herein. Rather, the invention should be construed to include any antigenic target that is associated with a disease where a universal immune receptor can be used to treat the disease.

The universal immune receptor-modified T cells of the invention may also serve as a type of vaccine for ex vivo immunization and/or in vivo therapy in a mammal. Preferably, the mammal is a human.

With respect to ex vivo immunization, at least one of the following occurs in vitro prior to administering the cell into a mammal: i) expansion of the cells, ii) introducing a nucleic acid encoding a universal immune receptor to the cells, and/or iii) cryopreservation of the cells.

Ex vivo procedures are well known in the art and are discussed more fully below. Briefly, cells are isolated from a mammal (preferably a human) and genetically modified (i.e., transduced or transfected in vitro) with a vector expressing a universal immune receptor disclosed herein. The universal immune receptor-modified cell can be administered to a mammalian recipient to provide a therapeutic benefit. The mammalian recipient may be a human and the universal immune receptor-modified cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present invention also provides compositions and methods for in vivo immunization to elicit an immune response directed against an antigen in a patient.

Generally, the cells activated and expanded as described herein may be utilized in the treatment and prevention of diseases that arise in individuals who are immunocompromised. In particular, the universal immune receptor-modified T cells of the invention are used in the treatment of cancer. In certain embodiments, the cells of the invention are used in the treatment of patients at risk for developing cancer. Thus, the present invention provides methods for the treatment or prevention of cancer comprising administering to a subject in need thereof, a therapeutically effective amount of the universal immune receptor-modified T cells of the invention.

The SpyTag or SpyCatcher universal immune receptor-modified T cells of the present invention may be administered either alone, or as a pharmaceutical composition in combination with diluents and/or with other components such as IL-2 or other cytokines or cell populations. Briefly, pharmaceutical compositions of the present invention may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, preferably 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently redraw blood (or have an apheresis performed), activate T cells therefrom according to the present invention, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocol may serve to select out certain populations of T cells.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the T cell compositions of the present invention are administered to a patient by intradermal or subcutaneous injection. In another embodiment, the T cell compositions of the present invention are preferably administered by i.v. injection. The compositions of T cells may be injected directly into a tumor, lymph node, or site of infection.

In certain embodiments of the present invention, cells activated and expanded using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The dosage of the above treatments to be administered to a patient will vary with the precise nature of the condition being treated and the recipient of the treatment. The scaling of dosages for human administration can be performed according to art-accepted practices. Strategies for T cell dosing and scheduling have been discussed (Ertl et al, 2011, Cancer Res, 71:3175-81; Junghans, 2010, Journal of Translational Medicine, 8:55).

### Screening

In one embodiment, the present invention provides a method for screening potential antigen binding compositions, including for example, antibodies, peptides, oligonucleotides, ribozymes, aptamers, and the like. According to one embodiment of the present invention, a T cell modified to express a universal immune receptor comprising a SpyTag or SpyCatcher binding domain is used to screen reciprocally tagged compositions (i.e tagged with a SpyCatcher or SpyTag moiety respectively) for the ability of the composition to bind to the target antigen. In one embodiment, the screening system of the invention is used for diagnostics, to determine patient eligibility on trial, to determine time of maximal binding to antigen in target tissue (without residual agent in healthy tissues), and as a means of monitoring response to therapy. In one embodiment, a cell based assay comprising the universal immune receptor-expressing modified T cell is used to screen compositions. In one embodiment, the assay comprises administering a tagged composition to the assay and detecting a detectable response induced by the T cell. For example, in one embodiment, the assay comprises detecting the activation of the T cell. In another embodiment, the assay comprises detecting the level of secreted cytokines. In one embodiment, the target antigen, for which an antigen binding composition is sought, is immobilized on a surface, for example a cell culture plate or a bead. In another embodiment, the assay comprises a cell expressing the target antigen.

### Methods of Quantifying Universal Immune Receptor (UIR) Turnover

In one aspect, a method of quantifying turnover of a universal immune receptor on a cell surface is provided. Because binding of SpyCatcher and SpyTag is covalent, it is possible to evaluate how quickly an armed universal immune receptor turns over on the cell surface. Knowledge of the rate of turnover could be useful in, for example, defining antibody dose strategies or regimens for patients.

In one embodiment, the method includes the steps of (a) contacting a cell genetically modified to express a universal immune receptor comprising a T cell receptor intracellular signaling domain, a transmembrane domain, and an extracellular domain comprising a SpyCatcher or a SpyTag with an agent comprising a SpyCatcher or SpyTag, thereby generating an armed receptor; and (b) determining an amount of the armed receptor relative to a reference amount. In one embodiment, the reference amount is an amount of the armed receptor at a prior time.

In an embodiment, a mammal has been administered, either sequentially or simultaneously, with the cells and the agent, and the mammal is monitored to determine the level of armed receptor. If the level of armed receptor is determined to be insufficient to treat the tumor or cancer, the mammal is administered with further cells and/or agent.

In one embodiment, the agent is linked to a SpyTag. In another embodiment, the agent is an antibody, an antibody fragment, a scFv, or a DARPin. In another embodiment, the agent is an antibody and is human IgG.

In one embodiment, the SpyTag or SpyCatcher is linked to the agent via light activated site-specific conjugation (LASIC). In one embodiment, the cell is a T cell, a Natural Killer (NK) cell, a cytotoxic T lymphocyte (CTL), a macrophage, a stem cell, or a regulatory T cell. In one embodiment, the cell is a T cell. In another embodiment, the cell is an autologous cell. In another embodiment, the universal immune receptor further comprises an intracellular domain of a costimulatory molecule. In some embodiments, the costimulatory molecule is 4-1BB, CD28, CD27, CD2, CD3, OX40, CD30, CD40, PD-1, ICOS, lymphocyte function-associated antigen-1 (LFA-1), CD7, LIGHT, NKG2C, B7-H3, or a ligand that specifically binds with CD83.

In one embodiment, the amount of the armed receptor is determined by labeling the agent and detecting the labeled agent. The agent can be labeled by linking or contacting the agent with a labeling molecule, where the labeling molecule includes, for example, a myc-tag, FLAG-tag, His-tag, HA-tag, fluorescent protein (e.g. green fluorescent protein (GFP)), a fluorophore (e.g. tetramethylrhodamine (TRITC), fluorescein isothiocyanate (FITC)), dinitrophenol, peridinin chlorophyll protein complex, green fluorescent protein, biotin, phycoerythrin (PE), histidine, streptavidin, avidin, horse radish peroxidase, palmitoylation, nitrosylation, alkalanine phosphatase, glucose oxidase, Glutathione S-transferase (GST), maltose binding protein, any types of fluorescent materials including quantum dot nanocrystals, radioisotopes, a heavy metal, a supramagnetic nanoparticle, and any types of compounds used for radioisotope labeling including, 1,4,7,10-tetraazacyclododecane-1,4,7,10-tetraacetic acid (DOTA), diethylene triamine pentaacetic acid (DTPA), 1,4,7-triazacyclononane-1,4,7-triacetic acid (NOTA), or allophycocyanin (APC).

Labeling of the agent with any of such labels may be performed directly or indirectly. The label may be conjugated to the molecule using techniques such as chemical coupling and chemical cross-linkers. Alternatively, polynucleotide vectors can be prepared that encode the labeled molecules as fusion proteins. Cell lines can then be engineered to express the labeled molecule, and the labeled molecule can be isolated from culture media, purified and used in the methods disclosed herein. A labeled amino acid, labeled peptide, labeled protein, or molecular reporter may also be ligated to the molecule via expressed protein ligation (e.g. using sortase, inteins, etc.).

In one embodiment, the amount of the armed receptor is determined by mass cytometry.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The materials and methods employed in these experiments are now described.

### Cloning of A24-Protein G-SpyTag (A24-PGST), SpyCatcher, and DARPin-SpyTag Constructs

The HTB1 domain of Protein G containing an amber codon in the 24th amino acid position, fused to a 7×GGS linker and SpyTag, was cloned into the pSTEPL vector via the NdeI and AgeI cloning sites as detailed in previous studies (Warden-Rothman et al., Anal Chem 2013, 85 (22), 11090-11097; Hui et al., Bioconjugate Chemistry 2015, 26 (8), 1456-1460). myc-DARPin9.26-SpyTag, myc-Eo1-SpyTag, and myc-Ec1-SpyTag were synthesized (GeneArt) and cloned in the pSTEPL vector via NdeI and AgeI cloning sites (Kasaraneni, N., mBio 2017, 8 (6), e01860-17; Steiner et al. Journal of Molecular Biology 2008, 382 (5), 1211-1227; Stefan et al. Mol Biol 2011, 413 (4), 826-843). To generate Flag-Eo1-SpyTag, polymerase chain reaction (PCR) extension was used to add an NdeI cut site and Flag-tag at the 5′ end of the Eo1-SpyTag construct. Flag-Eo1-SpyTag was then cloned into the pSTEPL vector following the methods detailed above. Myc-RFP-SpyTag was generated via PCR amplification of RFP using primers to add an NdeI cut site and myc-tag at the 5′ end, and XhoI cut site at the 3′ end, and was subsequently cloned into the pSTEPL vector upstream of a 7×GGS-SpyTag using the added cut sites. SpyCatcher-Venus was generated via PCR amplification of Venus using primers to add an NheI cut site at the 5′ end and XhoI cut site at the 3′ end, and was subsequently cloned into the pSTEPL vector downstream of a SpyCatcher-7×GGs domain using the added cut sites.

To generate SpyTag-DA versions of the above constructs, QuickChange Site-Directed mutagenesis (Agilent) was used, along with a forward (5′-GCATATCGTTATGGTCGCTGCTTACAAGCCAACGA-3′) and reverse (5′-TCGTTGGCTTGTAAGCAGCGACCATAACGATATGC-3′) primer to introduce an A to C point mutation in the SpyTag coding region, mutating Asp117 to Ala117 (Zakeri et al., Proc. Natl. Acad. Sci. U.S.A. 2012, 109 (12), E690-7).

All plasmids sequences were sequence verified.

### Expression and Purification of Bacterially Expressed Proteins

The pSTEPL plasmid containing the A24-Protein G-SpyTag (A24-PGST) sequence and pEVOL-pBpF were co-transformed into T7 Express Competent E. coli (New England Biolabs). Bacterial starter cultures in lysogeny broth (LB) with 100 mg/ml ampicillin and 25 mg/ml chloramphenicol were grown for 8 hours in a shaking incubator (230 rpm, 37 C). Starter cultures were then diluted 1:1000 in auto induction media (Formedium) containing 100 μmg/ml ampicillin, 25 μmg/ml chloramphenicol, 500 μM L-benzoylphenylalanine (Bachem), 0.1% w/v L-(+)-arabinose (Sigma-Aldrich), and 0.5% v/v glucose and grown for 16 hours in a shaking incubator (230 rpm, 37 C).

All pSTEPL plasmids containing DARPin and RFP constructs were transformed into T7 Express Competent E. coli (New England Biolabs). Cultures were grown as stated above in auto induction media (Formedium) containing 100 mg/ml ampicillin and 0.5% v/v glucose.

Cultures were pelleted at 7,000 rpm for 10 min. Pellets were resuspended in B-PER lysis buffer supplemented with 0.4 mg/mL lysozyme, 4 μg/mL DNase, and EDTA-free cOmplete protease inhibitor cocktail (Roche) at a ratio 5 mL buffer per 0.8-1 gram of pellet. Lysate was rotated at room temperature for 1 hour, followed by a 30 min freeze at −80 C. Cell lysate was then thawed at room temperature, followed by centrifugation at 15,000×g for 10 min at 4 C. The aqueous layer of the lysate was collected and incubated for 1 hour at room temperature in a 10 mL Poly-Prep chromatography column containing Talon 50/50 metal affinity resin (1 ml 50/50 resin/100 mL bacterial culture; Clontech). Lysate was allowed to pass through the column and the resin beads were washed with 5 column volumes of sterile 1×PBS without Magnesium and calcium (Corning).

Sortase cleavage purification was run as previously described (Warden-Rothman et al. Anal Chem 2013, 85 (22), 11090-11097). Briefly, 1×PBS containing 2 mM triglycine and 50 μM calcium chloride was added to each column (500 μL buffer per 1 mL 50/50 resin) and incubated for 2 hours at 37 C. Elutions were collected, filter sterilized, and stored at 4 C for later use.

A BCA assay kit (Pierce) was used to determine protein concentration. To test functionality of the SpyTag domain proteins were mixed with an excess of SpyCatcher-Venus and incubated for 30 minutes at room temperature. Samples were run on SDS-PAGE reducing gels (Bio-Rad) to confirm isopeptide bond formation. For in vivo studies, endotoxin was removed using a 1% Triton X-114 phase separation method (Liu et al., Clin Biochem 1997, 30, 455-463). Endotoxin levels were confirmed to be <10 endotoxin units/mL using the Endosafe®-PTS system and limulus amebocyte lysate (LAL) test cartridges (Charles River Laboratories).

### Light Activated Site-Specific Conjugation of A24-Protein G-SpyTag to Clinical Grade IgGs

A24-Protein G-SpyTag (A24-PGST) was crosslinked to clinical grade IgGs (trastuzumab, rituximab, cetuximab) following methods previously described by Hui, et al (Hui et al., Bioconjugate Chemistry 2015, 26 (8), 1456-1460). Briefly, after A24-PGST purification small pilot conjugation tests were conducted to determine the volume of A24-PGST needed to fully crosslink 1 μg of IgG. Conjugation was run for 2 hours at 4 C under UV light, and samples were run on SDS-PAGE reducing gels (Bio-Rad) to confirm conjugation of the IgG heavy chains with A24-PGST. The volume/weight ratio determined to achieve maximal conjugation was then used for large batch production following the same steps.

To remove excess A24-PGST, samples were centrifuged in 100 kDa MWCO spin filtration columns (Millipore Sigma) at 14,000×g, followed by a 5× column volume wash with 1×PBS. Protein concentration was determined via BCA assay kit (Pierce). Samples were stored at 4 C for later use.

### SDS-PAGE

Sodium dodecyl sulfate polyacrylamide gel electrophoresis was performed to determine expression of proteins, crosslinking of ProteinG-SpyTag and IgG heavy chains, and covalent bond formation between SpyTag-labeled targeting ligands and SpyCatcher reagents. Samples were boiled at 95 C for 5 minutes in the presence of 2-mercaptoethanol and loaded onto 4-15% gradient Tris/glycine gels (Bio-Rad). Gels were stained for 1 hour with SimplyBlue SafeStain (Invitrogen), followed by overnight destaining with water.

### Cloning of Lentiviral Constructs and Lentiviral Packaging

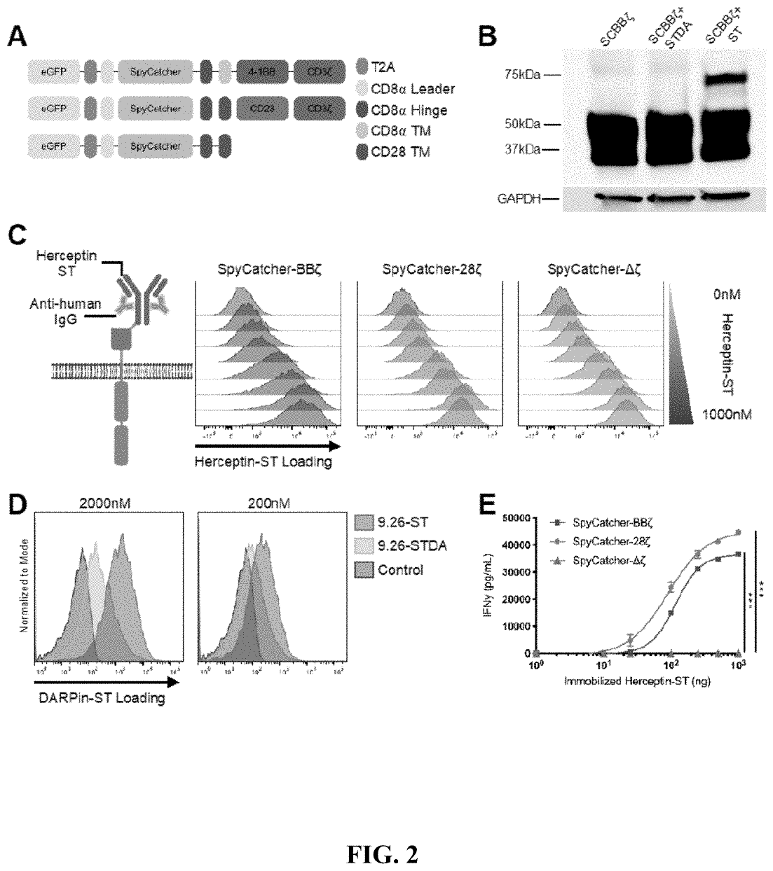

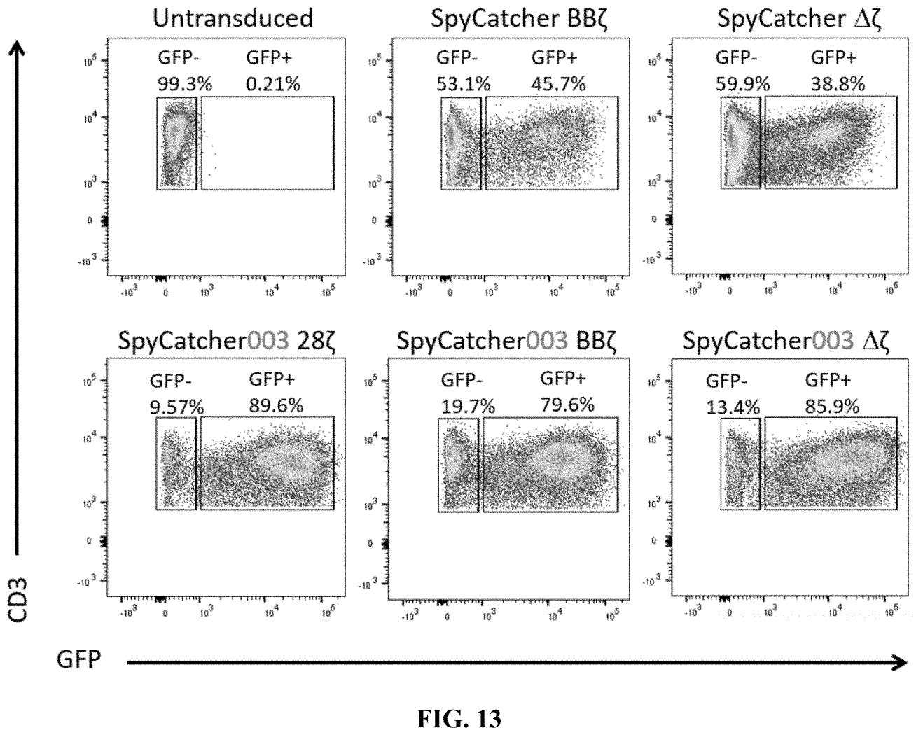

A gene fragments encoding the truncated version of SpyCatcher (SpyCatcherΔ) (Li et al., J. Mol. Biol. 2014, 426 (2), 309-317) was synthesized (GeneArt) and digested with restriction enzymes BamHI and NheI. The insert was ligated into a third generation replication-incompetent lentiviral vector containing either CD28-CD3ζ, 41BB-CD3ζ, or lacking functional intracellular T cell signaling domains (Delta-Zeta; Δζ) intracellular domains. A region encoding GFP and a T2A site were upstream of the receptor, allowing for detection of transgene expression using GFP as a surrogate marker (FIG. 2A and FIG. 13). The same methodology was used to clone anti-Her2 chimeric antigen receptors using the Herceptin derived scFv (4D5) (Zhao, 2009) containing either CD28-CD3ζ or 41BB-CD3ζ intracellular signaling domains. Expression of all receptors was driven by the EF1a promoter (Milone et al., Molecular Therapy 2009, 17 (8), 1453-1464).

HER2 WT was a gift from Mien-Chie Hung (Addgene plasmid #16257; http://n2t.net/addgene:16257; RRID:Addgene_16257) (Li et al., Cancer Cell 2004, 6 (5), 459-469). EGFR WT was a gift from Matthew Meyerson (Addgene plasmid #11011; http://n2t.net/addgene:11011; RRID:Addgene_11011) (Greulich et al., Plos Med 2005, 2 (11), e313). The Her2 and EGFR sequences were amplified via PCR and digested with the restriction enzymes XbaI and SalI. Gene sequences were ligated into a third-generation replication-incompetent lentiviral packaging vector downstream of an EF1a promoter.

Replication-incompetent lentivirus was produced using HEK293T cells (Invitrogen) as previously described (Smith et al., Molecular Therapy 2016, 24 (11), 1987-1999). Briefly, pELNS transfer plasmid and lentiviral packaging plasmid pVSV (VSV glycoprotein expression vector), pRSV.REV (Rev expression vector), and pMDLg/p.RRE (Gag/Pol expression plasmid) were transfected into HEK293T cells using Lipofectamine 2000 (Invitrogen). 24 hour and 48 hour supernatants were harvested and virus was pelleted via ultracentrifugation at 25,000 rpm for 2 hours. Concentrated virus was stored at −80 C until use. Viral titer (IU/mL) was determined using viral transduction of HEK293T cells through measurement of the surrogate marker GFP.

### Cell Lines

Established human tumor cell lines SKOV3, MDA-MB-468, A1847, CRL5803, MDA-MB-361, Ramos, and HEK293T were purchased from the American Type Culture Collection (ATCC). Cells were cultured in complete medium (CM) comprised of RPMI 1640 (GIBCO) supplemented with 10% FBS (VWR), 100 U/mL of penicillin, and 100 μm/mL of streptomycin, at 37 C and 5% CO2. All cell lines were routinely tested for mycoplasma.

To generate Ramos-Her2 and Ramos-EGFR lines, Ramos cells were transduced with lentivirus containing the coding sequence of either protein. Cells were then propagated, stained with either anti-Her2 (APC; BioLegend) or anti-EGFR antibody (PE; BioLegend), and sorted for protein expression using fluorescence activated cell sorting (FACS).

To generate SKOV3-SpyCatcher-BBζ, SKOV3 cells were transduced with lentivirus containing the coding sequence for the GFP-T2A-SpyCatcher-BBζ. Cells were propagated and sorted for GFP expression using FACS.

Healthy donor primary human T cells were purchased from the Human Immunology Core (University of Pennsylvania). CD4+ and CD8+ T cells were combined in equal amounts and stimulated with anti-CD3/CD28 beads (Invitrogen) at a 3:1 ratio. During the expansion process, T cells were maintained in CM at 37 C and 5% CO2. After 24 hours, lentivirus was added at a multiplicity of infection (MOI) of 5-10. Seven days after activation, the anti-CD3/CD28 beads were removed from culture via magnetic separation. T cells were cultured for an additional 7 days at a density of 0.5-1×106 cells/mL. CM was supplemented with 50-100-IU/mL IL-2 (Prometheus Therapeutics and Diagnostics) after lentivirus addition and maintained until 2 days before T cell use in functional assays. T cell count and volume were continually tracked during the expansion process using a Coulter Counter (Beckman Coulter). Transduction efficiency was detected by flow cytometry for the expression of GFP.

### SpyCatcher T Cell Arming and Detection of Armed Receptors

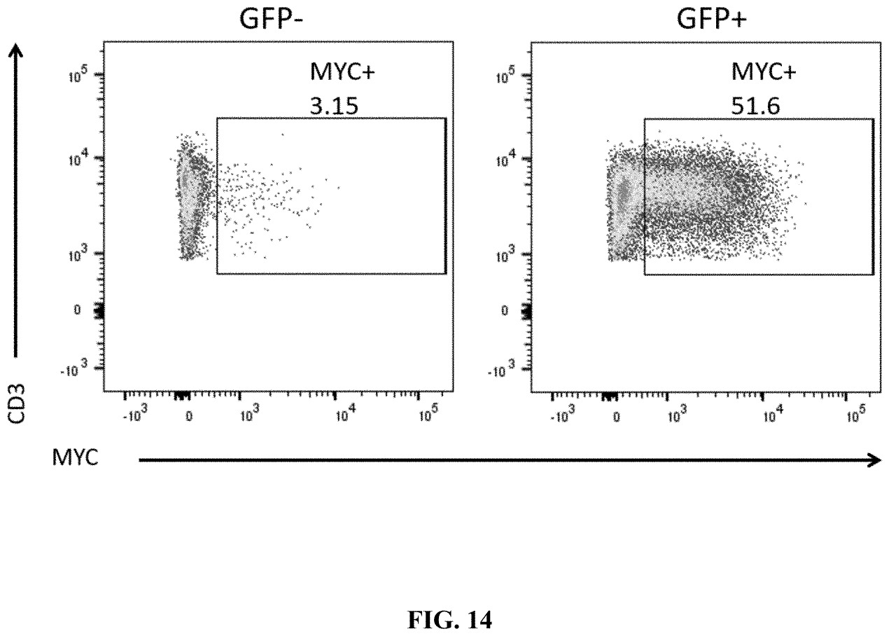

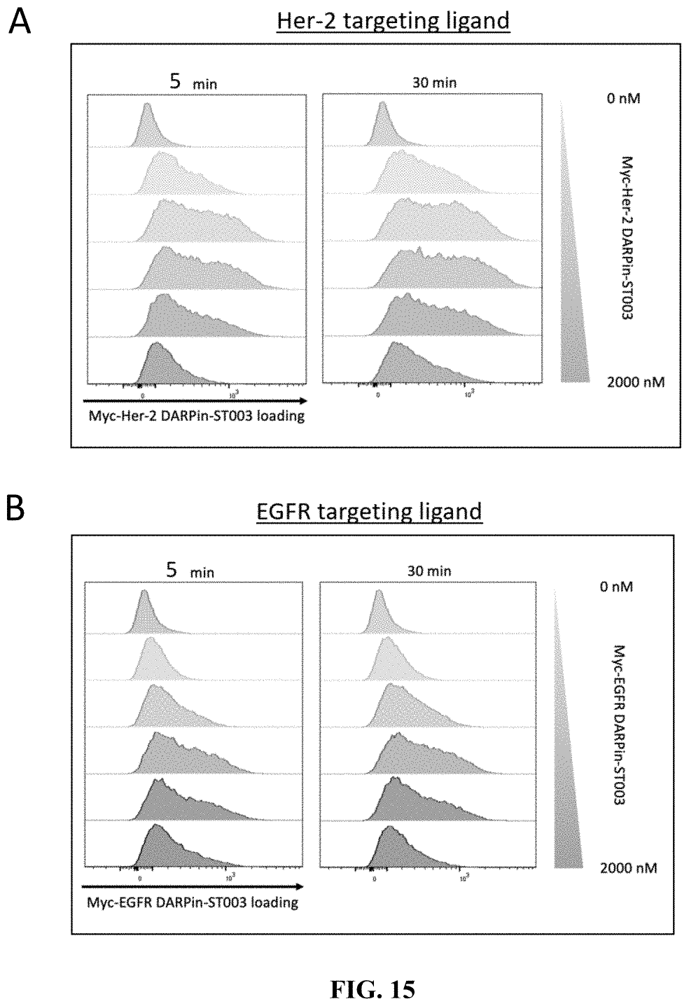

SpyCatcher expressing T cells were resuspended in CM containing various concentrations of SpyTag-labeled targeting agent. Cells were incubated for 30-60 minutes at 37 C and 5% CO2 and then washed 2× with CM. For staining of IgG-SpyTag armed T cells, goat polyclonal anti-human IgG (Sigma-Aldrich) conjugated with LightningLink APC (Expedeon) was used. For staining of myc- or Flag-DARPin-SpyTag armed T cells, either anti-Myc-Alexa647 (Cell Signaling Technologies) or anti-FLAG-BV421 (BioLegend) were used. Stained cells were analyzed by flow cytometry (FIG. 14 and FIG. 15).

For receptor turnover experiments, T cells were expanded and rested to an average volume of <300 fL and then armed with either SpyTag-labeled IgG or SpyTag-labeled DARPin, as described above. For basal turnover, T cells were maintained in CM and stained for loaded receptor every 24 hours for a total of 96 hours. For antigen induced turnover, T cells were combined at a 3:1 effector to target ratio and stained after 24 hours in culture.

### Western Blot

SKOV3-SpyCatcher-BBζ were incubated with either 2000 nM myc-RFP-SpyTag or myc-RFP-SpyTag-DA for 1 hour at 37 C. Cells were lysed using RIPA lysis buffer with Protease inhibitor cocktail (Roche, Cat #5892970001) and centrifuged for 5 minutes. Lysate was then collected and protein concentrations were quantified using BCA assay (Thermo Scientific). 80 μg of protein samples were mixed with loading buffer (Lammeli buffer; BioRad) containing 5% β-mercaptoethanol (BioRad) and incubated at 95 C for 5 minutes. Samples were loaded in 4-15% Minigels protean TGX (BioRad) and run at 150V for 1 hr. Protein ladder (BioRad) was run along with the samples. Protein samples were transferred to PVDF membrane (Millipore) at 100V for 1 hr. The membranes were washed with TBST (1% Tween) (BioRad) and incubated with primary and secondary antibodies, including Purified Mouse Anti-Human CD3ζ (BD Pharmigen; 1:1,000), anti-Human/Mouse/Rat GAPDH, (R&D; 1:20,000), and peroxidase AffiniPure Goat anti-Mouse IgG (Jackson Immunology; 1:10,000). Membranes were washed three times in between the primary and secondary antibodies incubation steps. Membranes were developed using the ECL prime western blotting detection reagent (GE Healthcare #RPN2236) and imaged using GE ImageQuant LAS 4000 series imaging system.

### Testing Effector Functions of SpyCatcher T Cells

For cytokine secretion assays, Herceptin-ST proteins were diluted in coating buffer (BioLegend) and incubated in flat bottom MaxiSorp plate (Sigma) overnight at 4 C. Wells were washed twice with PBS and 70,000 immune receptor-(+) cells were added to each well. Plates were covered with a breathable seal and incubated at 37 C and 5% CO2 for 16 hours. Harvested supernatant was stored at −20 C for later use. IFNγγ secretion levels were assessed using the human IFNγγ ELISA kit (BioLegend) following the included protocol.

End point testing of lytic function was done using Luc-Screen™ Extended-Glow Luciferase Reporter Gene Assay System (Applied Biosystems). For armed SpyCatcher T cell lysis, SpyCatcher T cells were incubated with precise concentrations of the appropriate SpyTag-labeled targeting ligand as described above. Armed SpyCatcher T cells and click beetle green luciferase (CBG) expressing tumor cells were combined at a 7:1 E:T ratio in 200 uL phenol-free CM and incubated overnight at 37 C and 5% CO2. Plates were centrifuged at 1,200 rpm for 5 minutes, 100 μL of media was removed from each well, and luciferase buffer was added per the manufacturer's protocol. Luciferase readings were obtained using a microplate reader. For on-demand lysis, SpyCatcher T cells, targeting ligand, and tumor cells were combined simultaneously in phenol-free CM. All other steps were carried out in the same manner as armed lysis. Cytotoxicity was calculated using the following equation: [1−(T cells+targeting ligand+target cell)/(target cells alone)−1−(T cells+target cell)/(target cells alone)]*100.

Real-time lysis analysis was carried out using the xCELLigence Real Time Cell Analysis instrument (ACEA Biosciences). Adherent tumor cells were plated and incubated in the instrument overnight at 37 C with 5% CO₂. SpyCatcher T cells were then added after arming as described above. For on-demand lysis, SpyCatcher T cells were added to the tumor cells and incubated for 4 hours, followed by targeting ligand addition. Cytotoxicity calculations were done using the RTCA software (ACEA Biosciences).

### Xenograft Models

NOD-scid IL2Rγγnull were purchased from the Stem Cell and Xenograft Core (University of Pennsylvania). 6-12 week old female mice were kept in a pathogen-free environment following protocols approved by the University of Pennsylvania Institutional Animal Care and Use Committee. For intraperitoneal (I.P.) tumor models, mice were injected I.P. with 1×106 SKOV3-CBG+GFP tumor cells. After 7 days, 12.5×106 SpyCatcher immune receptor T cells were armed with 1000 nM Herceptin-ST and injected I.P. SpyTag-labeled targeting ligand was injected I.P. one day post T cell injection, followed by subsequent injections every 3 days until treatment cessation. Tumor progression was measured via bioluminescence imaging as described previously (Lanitis et al., Molecular therapy: the journal of the American Society of Gene Therapy 2012, 20 (3), 633-643) and quantified as average radiance, gated on the abdomen between the fore and hind limbs. Mice were sacrificed upon gaining 20% body weight due to ascites formation. Mice that formed palpable subcutaneous (S.C.) tumor at site of injection by day 11 post tumor injection were excluded from all groups.

Blood samples were collected via retro-orbital bleeds by the Stem Cell and Xenograft Core (University of Pennsylvania). Peripheral blood T cells were quantified using Trucount™ Tubes following the provided protocol (BD Biosciences). Staining panel consisted of anti-human CD3 (Brilliant Violet 605; BioLegend), anti-human CD45 (PE; eBiosciences), and anti-human CD8 (APC-H7; BD Biosciences).

For S.C. tumor models, mice were subcutaneously injected in the flank with 1×106 SKOV3-CBG+GFP tumor cells. After 6 days, 12.5×106 SpyCatcher immune receptor T cells were armed and I.P. injected. SpyTag-labeled targeting ligand was I.P. injected one day after T cell infusion, followed by subsequent injections every 3 days until treatment cessation. Tumor progression was measured via caliper measurement and quantified using the following formula: volume=3.14/6(length*(width)2), with length being the longest diameter and width being the shortest diameter.

### Statistical Analysis

Data are reported as mean+/−standard deviation (SD) unless otherwise noted. Statistical analysis was performed using unpaired 2-tailed t test unless otherwise noted. GraphPad Prism 8.0 software was used for statistical analysis. P<0.05 was considered significant.

### Videos

GFP-SpyCatcher-BBζ T cells armed with 1000 nM (V1), 100 nM (V2), 10 nM (V3), or 0 nM (V4) of Herceptin-ST demonstrate dose-dependent lysis of Her2+SKOV3 cells expressing nuclear RFP. Tumor cell lysis was not observed by GFP-SpyCatcher-Δζ T cells armed with 1000 nM (V5) Herceptin-ST. Addition of Herceptin-ST to unarmed GFP-SpyCatcher-BBζ T cells co-cultured with Her2+SKOV3 cells induces lytic function (V6; Herceptin-ST added at 48 hrs).

The results of the experiments are now described.

### Generation of SpyTag-Labeled Tumor Antigen Targeting Ligands

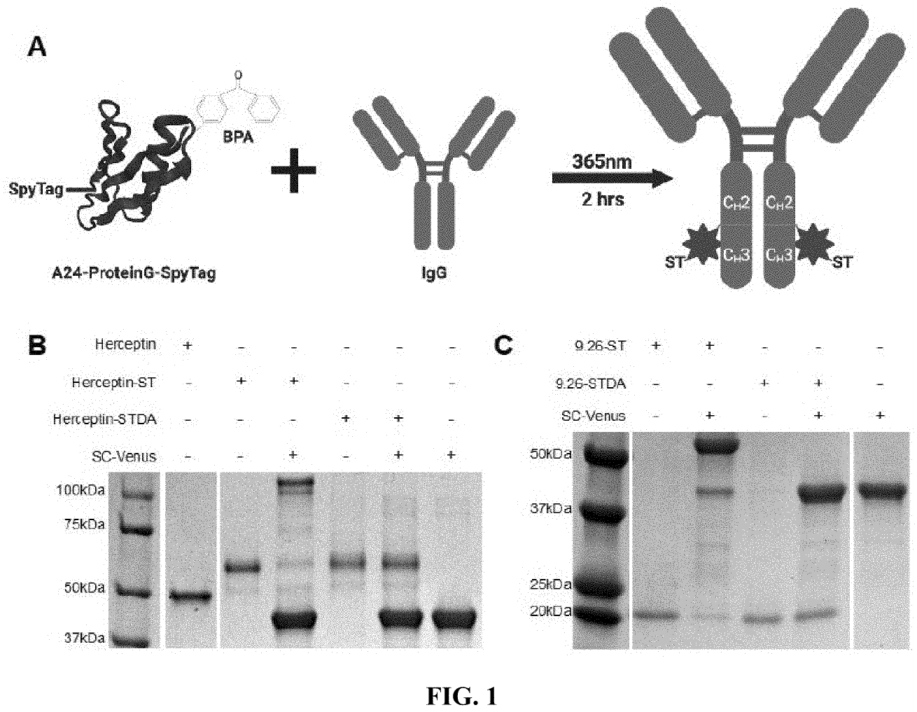

The SpyCatcher immune receptor system is comprised of two main components: targeting ligands containing a SpyTag domain and T cells expressing the SpyCatcher immune receptor (FIG. 1A). We used two approaches to generate SpyTag-containing targeting ligands: site-specific conjugation and genetic fusion. Site-specific attachment of SpyTag to clinical grade IgGs was achieved using a light activated site-specific conjugation (LASIC) adaptor protein developed by Hui et al Bioconjugate Chemistry 2015, 26 (8), 1456-1460. This adaptor protein is derived from Protein G, an IgG-binding bacterial cell wall protein that interacts with the Fc portion of human IgGs at the CH2-CH3 junction (Sauer-Eriksson et al., Struct Lond Engl 1993 1995, 3 (3), 265-278). Using an amber-tRNA suppressor aminoacyl-synthase pair, Protein G-SpyTag was expressed with the unnatural amino acid benzoylphenylalanine (BPA) incorporated at the A24 amino acid position. When combined together with IgGs, A24-Protein G-SpyTag binds to the Fc-domain of the IgG non-covalently. Upon exposure to UV light (365 nm), the BPA molecule is activated and covalently cross-links the Protein G-SpyTag site-specifically to the Fc domain (FIG. 1A). The final IgG molecule thus contains two covalently linked Protein G-SpyTag molecules, one on each side of the Fc domain (FIG. 1A).

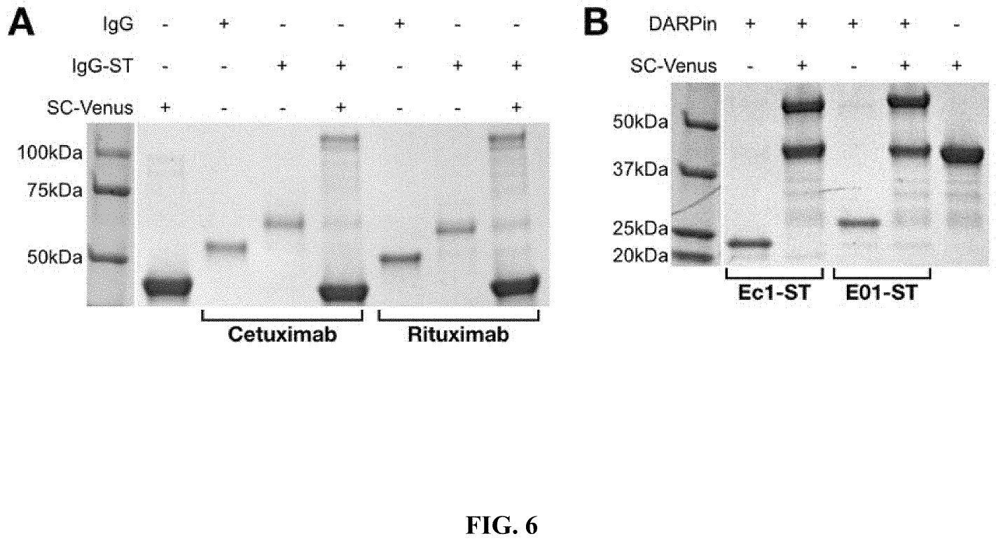

Analysis of clinical grade antibodies crosslinked with Protein G-SpyTag by reducing SDS-page gel demonstrated nearly full crosslinking of the IgG heavy chain for Herceptin (FIG. 1B), cetuximab, and rituximab as demonstrated by a band shift equal to the combined masses of the two proteins (˜60 kDa combined) (FIG. 6A). To demonstrate maintained functionality of the SpyTag domain after UV exposure, we incubated Herceptin-SpyTag with an excess of SpyCatcher-Venus (38 kDa). The formation of a shifted band that was maintained under boiling and reducing conditions at the approximate combined mass of both proteins (100 kDa) was observed, as well as the loss of the single heavy chain-Protein G band, indicating the formation of a covalent bond between the two proteins (FIG. 1B). In order to further broaden the repertoire of targeting ligand types, Her2 (DARPin9.26; 22.5 kDa) (Kasaraneni et al., mBio 2017, 8 (6), e01860-17) (FIG. 1C), EGFR (E01; 22.4 kDa) (Steiner et al., Journal of Molecular Biology 2008, 382 (5), 1211-1227), or EpCAM (Ec1; 22.8 kDa) (Stefan et al., J Mot Biol 2011, 413 (4), 826-843) (FIG. 6B) targeting designed ankyrin repeat proteins (DARPins) containing a C-terminal SpyTag domain were expressed using the sortase-tag expressed protein ligation (STEPL) system (Warden-Rothman et al., Anal Chem 2013, 85 (22), 11090-11097). SpyTag functionality was again validated via conjugation with excess Venus-SpyCatcher, demonstrating the formation of a shifted band resistant to degradation under boiling and reducing conditions at the approximate combined molecular weights of the two protein components (61 kDa) (FIG. 1C; FIGS. 6A-6B).

Previous studies have demonstrated that reaction of a SpyTag variant containing an aspartic acid to alanine mutation (SpyTag-DA) abolishes covalent bond formation with SpyCatcher, while still allowing for the formation of a noncovalent complex with a Kd of 200 nM (Zakeri et al., Proc. Natl. Acad. Sci. U.S.A. 2012, 109 (12), E690-7). To serve as a negative control for covalent bond formation, Herceptin-STDA and 9.26-STDA targeting ligand were also produced (FIGS. 1B-1C).

### Cloning, Expression, and Detection of SpyCatcher Immune Receptor

The truncated, 84 amino acid version of SpyCatcher (Li et al., J. Mol. Biol. 2014, 426 (2), 309-317) was cloned into previously validated (Lanitis et al., Molecular therapy: the journal of the American Society of Gene Therapy 2012, 20 (3), 633-643; Song et al., Human gene therapy 2013, 24 (3), 295-305) lentiviral constructs containing either 4-1BB or CD28 costimulatory domains in tandem with CD3ζ to produce SpyCatcher-BBζ and SpyCatcher-28ζ immune receptors. A SpyCatcher receptor lacking intracellular signaling domains was also generated to serve as a negative control (SpyCatcher-Δζ; FIG. 2A). All constructs contained eGFP upstream of a T2A self-cleaving peptide, allowing for the co-expression of GFP and the receptor, and the use of GFP as a surrogate marker for primary human T cell transduction.

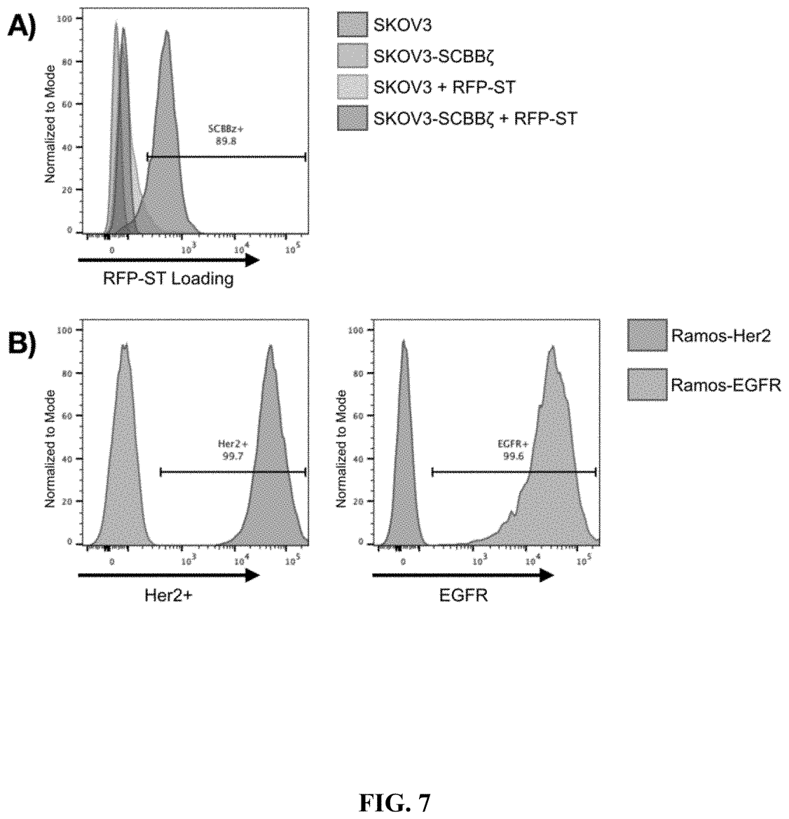

To determine whether SpyTag can covalently link to a SpyCatcher immune receptor expressed on the cell surface, an immortalized cancer cell line, SKOV3, was transduced to express the SpyCatcher-BBζ construct (FIG. 7A) and then incubated with soluble RFP-SpyTag protein. Incubation resulted in the formation of a shifted band that was resistant to degradation under reducing conditions when assessed via western blot staining for CD3ζ, confirming covalent bond formation between the two proteins (FIG. 2B). The remaining unshifted band between 50 kDa-37 kDa likely represent intracellular receptor, since whole cell lysate was loaded onto the gel. Incubation with RFP-SpyTag-DA protein showed that mutation of the critical aspartic acid residue in SpyTag to an alanine abolished its ability to form a covalent bond with SpyCatcher (FIG. 2B). Next, primary human T cells were transduced to express the SpyCatcher immune receptor and incubated with Herceptin-ST at various concentrations which resulted in dose-dependent loading (“arming”) of the receptor, as detected via staining with APC anti-human IgG polyclonal antibody (FIG. 2C). Additionally, covalent bond formation between the SpyTag-conjugated targeting ligand (9.26-ST) and the SpyCatcher receptor was necessary to achieve maximum targeting ligand arming, especially at low concentration (FIG. 2D).

### In Vitro Efficacy of SpyCatcher T Cells

In order to determine if SpyTag engagement of the SpyCatcher immune receptor on primary human T cells would induce specific activation, SpyCatcher T cells were incubated in the presence of various amounts of immobilized Herceptin-ST. Both SpyCatcher-BBζ and SpyCatcher-28ζ T cells, but not SpyCatcher-Δζ T cells, secreted IFNγγ in the presence of immobilized Herceptin-ST in a dose-dependent fashion (FIG. 2E), with SpyCatcher-28ζ T cells being more immunologically sensitive than SpyCatcher-BBζ T cells (P<0.001).

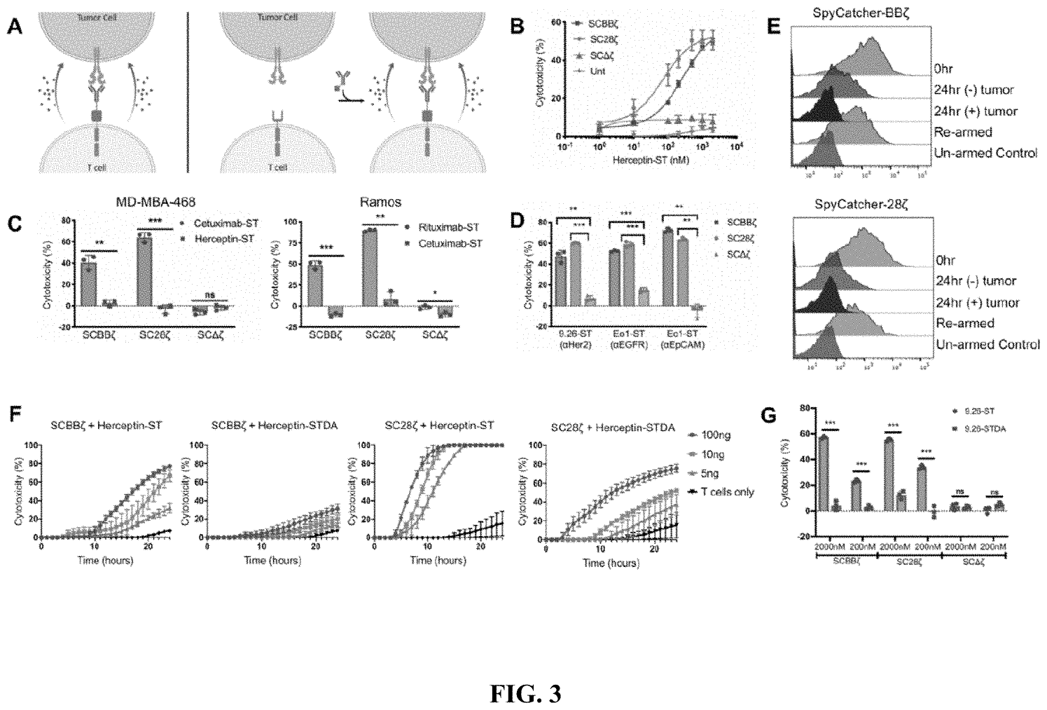

Since the SpyCatcher immune receptor is the first universal immune receptor that can be covalently armed with targeting ligands, permanently affixing antigen specificity to the receptor until it is degraded, we evaluated the lytic capabilities of pre-armed SpyCatcher T cells, in the absence of excess targeting ligand. It was sought to be determined if a sufficient amount of targeting ligand could be covalently armed onto the receptors at the cell surface to elicit T cell lysis of cancer cells upon antigen recognition (FIG. 3A, left). SpyCatcher-BBζ and SpyCatcher-28t armed with Herceptin-ST lysed Her2(+) SKOV3 tumor cells, while untransduced and SpyCatcher-Δζ T cells armed with Herceptin-ST showed minimal lytic activity after 20 hour co-culture (FIG. 3B). Lysis occurred in a dose dependent manner, with increasing Herceptin-ST arming concentration correlating with increasing lytic capacity (FIG. 3B), corresponding with the dose-dependent receptor loading observed previously (FIG. 2C). Observation of lytic function using live cell imaging confirmed these results, while also demonstrating arming dose dependent T cell clustering (Videos). Tumor cell lysis and T cell clustering was no observed with SpyCatcher-Δζ T cells armed with 1000 nM Herceptin-ST, demonstrating lysis and clustering are activation dependent.

Additionally, dose-dependent arming also impacted the ability of SpyCatcher T cells to recognize tumor cells expressing various levels of target antigen. SpyCatcher-BBζ T cells were capable of lysing tumor cells expressing high levels of antigen in an arming concentration dependent manner but lost functionality against cell lines expressing lower levels of Her2 (FIGS. 8A-8B). In contrast, highly armed SpyCatcher-28ζ T cells were capable of targeting tumor cells with variable Her2 expression, with lytic capacity correlating with the level of Her2 expression. When arming concentration was lowered the SpyCatcher-28ζ T cells lost efficacy against tumor cell lines expressing lower levels of Her2 (FIGS. 8A-8B).

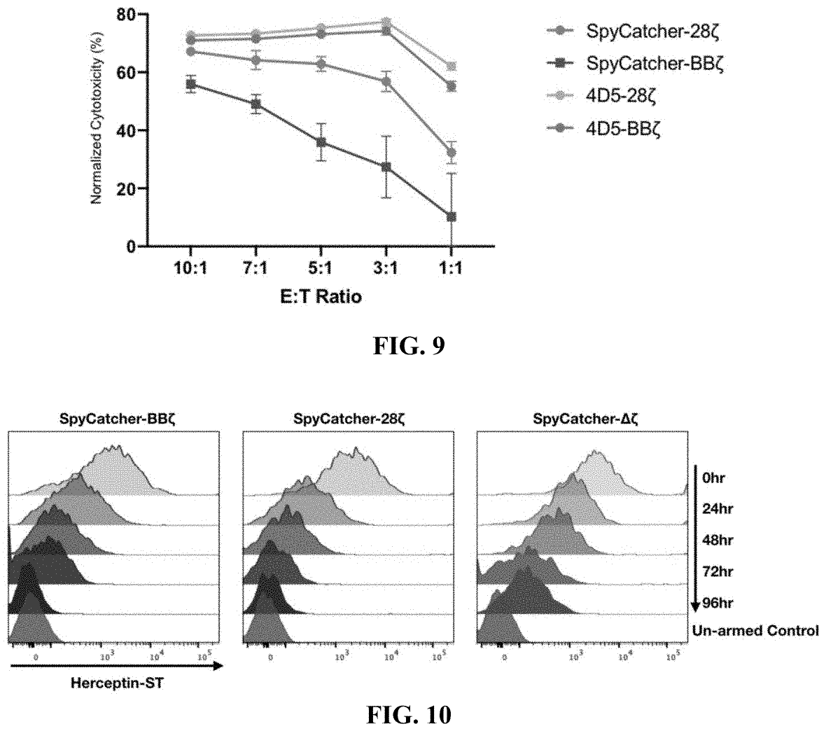

When compared to Her2-targeting chimeric antigen receptor T cells containing either 28ζ or BBζ intracellular signaling domains, it was found that SpyCatcher T cells maximally armed with Herceptin-ST were less effective than the CARs, particularly at lower E:T ratios (FIG. 9).

SpyCatcher-BBζ and SpyCatcher-28ζ were also able to lyse EGFR(+) or CD20(+) tumor cells when armed with the EGFR-targeting antibody Cetuximab-ST or the CD20-targeting antibody Rituximab-ST, respectively (FIG. 3C). The site-specific labeling method used for the off-the-shelf clinical grade antibodies led to the addition of a SpyTag peptide to both IgG heavy chains, as was previously shown by Hui et al. and further indicated by the nearly complete shift in the heavy chain band post-conjugation (FIG. 1B) (Hui et al., Bioconjugate Chemistry 2015, 26 (8), 1456-1460). This multivalent labeling method could potentially cause antigen-independent activation if two SpyCatcher immune receptors were crosslinked by the two SpyTag peptides on the same antibody, or if two SpyCatcher T cells were tethered with each other by SpyTag peptides on the same antibody. However, SpyCatcher T cells armed with the non-targeting control antibodies identically conjugated with two SpyTag peptides did not mediate significant lysis, demonstrating that bivalent SpyTag labeling of targeting ligands does not cause appreciable antigen-independent activation of T cells upon arming (FIG. 3C). Bivalently labeled antibodies were able to induce T cell activation when immobilized. Covalent loading of SpyTag containing designed ankyrin repeat proteins (DARPins) targeting the tumor antigens Her2, EGFR, or EpCAM also lead to specific lysis of antigen expressing tumor cells, demonstrating the potential to use multiple targeting ligand types with T cells bearing the SpyCatcher receptor (FIG. 3D).

Upon antigen recognition and CAR T cell activation, CARs are internalized, leading to lower detectable levels at the cell surface (Walker et al., Molecular Therapy 2017, 25 (9), 2189-2201). To evaluate the rate of armed SpyCatcher receptor loss on the T cell surface in the setting of antigenic stimulation, SpyCatcher T cells were armed, washed and co-cultured with or without antigen expressing tumor cells. In the absence of antigen expressing tumor cells, moderate armed receptor loss occurred, with SpyCatcher-28ζ T cells experiencing more rapid loss within 24 hours relative to SpyCatcher-BBζ T cells. Similar to CARs52, when stimulated with antigen expressing cancer cells, armed receptor expression was not detected on either SpyCatcher-BBζ or SpyCatcher-28ζ T cells at the same time point (FIG. 3E). However, both SpyCatcher-BBζ and SpyCatcher-28ζ T cells were amenable to re-arming, demonstrating that expression of SpyCatcher receptors by T cells is maintained and capable of binding newly introduced targeting ligand (FIG. 3E). In order to determine the rate of armed receptor loss from the cell surface in non-activated cells, SpyCatcher T cells were rested, then armed with Herceptin-ST, and subsequently analyzed for detectable, armed receptor via flow cytometry every 24 hours. Results show that armed receptor levels are gradually depleted over time, with full loss occurring approximately 96 hours after arming (FIG. 10).

Based on the finding that armed, but not unarmed, SpyCatcher T cells are capable of targeting and killing antigen expressing tumor cells, it was hypothesized that unarmed SpyCatcher T cell effector function could be temporally triggered upon later addition of targeting ligand to un-armed SpyCatcher T cells in co-culture with antigen expressing tumor cells (FIG. 3A, right). To test for “on-demand” cancer cell lysis, Unarmed SpyCatcher T cells were incubated with SKOV3 (Her2+/CD20−) tumor cells for 4 hours in the absence of targeting ligand. Addition of Herceptin-ST after 4 hours induced rapid tumor cell lysis in cultures containing either SpyCatcher-BBζ or SpyCatcher-28ζ T cells (FIG. 3F). Compared to SpyCatcher-BBζ T cells, SpyCatcher-28ζ T cells reacted more rapidly upon addition of Herceptin-ST, lysed target cells at lower targeting ligand concentrations, and reached a higher level of maximum lysis. Though equivalent levels of maximum lysis were achieved with SpyCatcher-28ζ T cells at different doses of Herceptin-ST, the initial lysis kinetics occurred in a dose dependent fashion (FIG. 3F). SpyCatcher-BBζ T cells displayed a more titratable response, with lower maximal lysis at lower Herceptin-ST doses.

### Testing the Effects of Covalent Bond Formation on Lytic Function

To test the effect of covalent bond formation on receptor arming and T cell activation, both “arming” and “on-demand” lysis experiments were performed comparing Herceptin-ST and Herceptin-STDA, or the anti-Her2 DARPins 9.26-ST and 9.26-STDA, respectively (FIGS. 3F and 3G). SpyCatcher-BBζ and SpyCatcher-28ζ T cells armed with 9.26-ST lysed Her2+SKOV3 tumor cells, while those armed with 9.26-STDA exhibited reduced or no lysis (FIG. 3G). This result corresponds with previous data demonstrating that covalent bond formation is necessary for maximal loading of the SpyCatcher receptor, particularly at lower arming concentrations (FIG. 2D). Covalent bond formation also impacted “on-demand” lysis. Herceptin-STDA addition led to slower lysis kinetics and lower maximal lysis compared to Herceptin-ST at equivalent doses (FIG. 3F). In all cases, SpyCatcher-28ζ T cells exhibited increased effector function, compared to SpyCatcher-BBζ T cells, when armed or co-cultured with excess Herceptin-STDA (FIGS. 3F and 3G).

### Simultaneous Receptor Arming and Dual Antigen Targeting

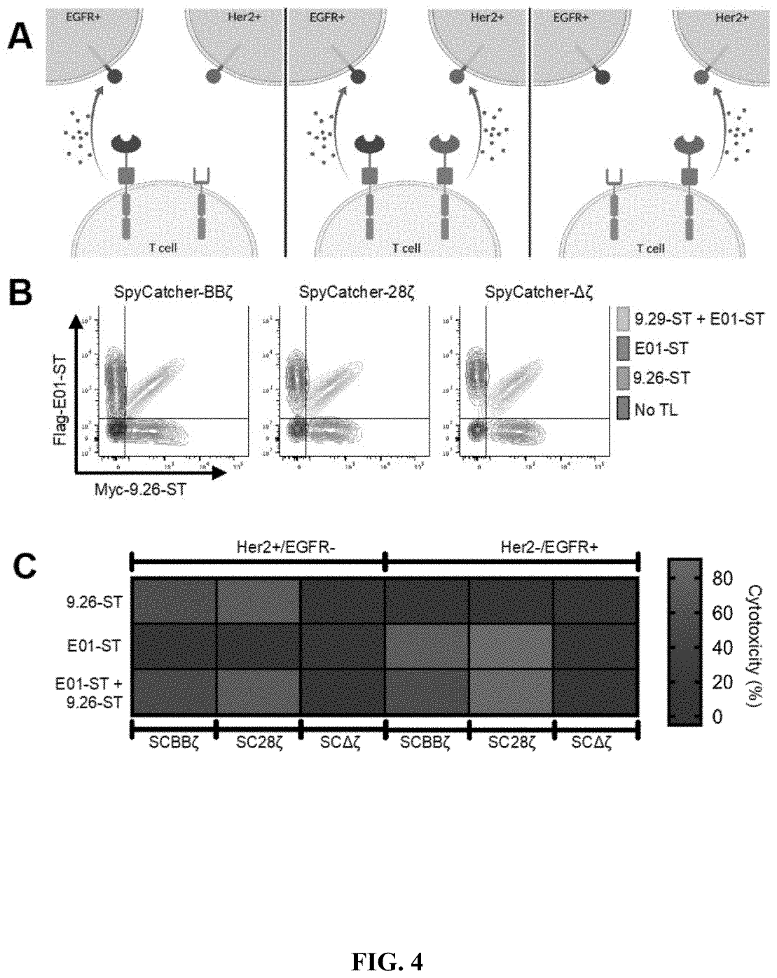

One advantage of covalent universal immune receptor loading is the ability to affix multiple targeting ligands with different specificities onto receptors at the T cell surface, creating a single cell product with the capability of targeting multiple antigens simultaneously (FIG. 4A). To test this, Her2-targeting (9.26-ST) and EGFR-targeting (E0o1-ST) DARPins containing unique tags for detection by flow cytometry were loaded onto SpyCatcher T cells either alone or in combination at a 1:1 molar ratio (FIG. 4B). Tag staining and flow cytometric analysis revealed that single DARPin loaded SpyCatcher T cells stained for only a single tag, while SpyCatcher T cells co-incubated with both DARPins displayed equivalent staining for each tag, and thus equivalent arming (FIG. 4B). These dual armed T cells were capable of lysing both Her2+/EGFR− and EGFR+/Her2-Ramos cells (FIG. 7B), while SpyCatcher T cells armed with a single DARPin targeting ligand were only capable of lysing cells expressing the prescribed target antigen. Levels of specific cell lysis mediated by dual armed T cells were similar to their single-armed counterparts, demonstrating the capacity of this single cell product to simultaneously target multiple antigens (FIG. 4C). SpyCatcher-Δζ T cells remained inactive regardless of the arming agent (FIG. 4C).

To assess the capability of combined arming enhancing SpyCatcher T cell function against a dual antigen expressing tumor line, SpyCatcher-BBζ T cells were armed with low doses of either 9.26-ST or E01-ST, or both in combination. When co-cultured with the Her2+/EGFR+SKOV3 tumor line (FIG. 8C), dual armed SpyCatcher-BBζ T cells achieved increased tumor lysis relative to singled armed cells alone (FIG. 8D).

### In Vivo Efficacy in Xenograft Tumor Models

We next tested the effectiveness of the SpyCatcher T cell system in xenograft tumor models using nonobese diabetic (NOD)-scid gamma (NSG) mice. Though our in vitro results showed that SpyCatcher-28ζ T cells displayed more potent effector function than SpyCatcher-BBζ T cells, we chose to move forward with pre-clinical models using the SpyCatcher-BBζ receptor.

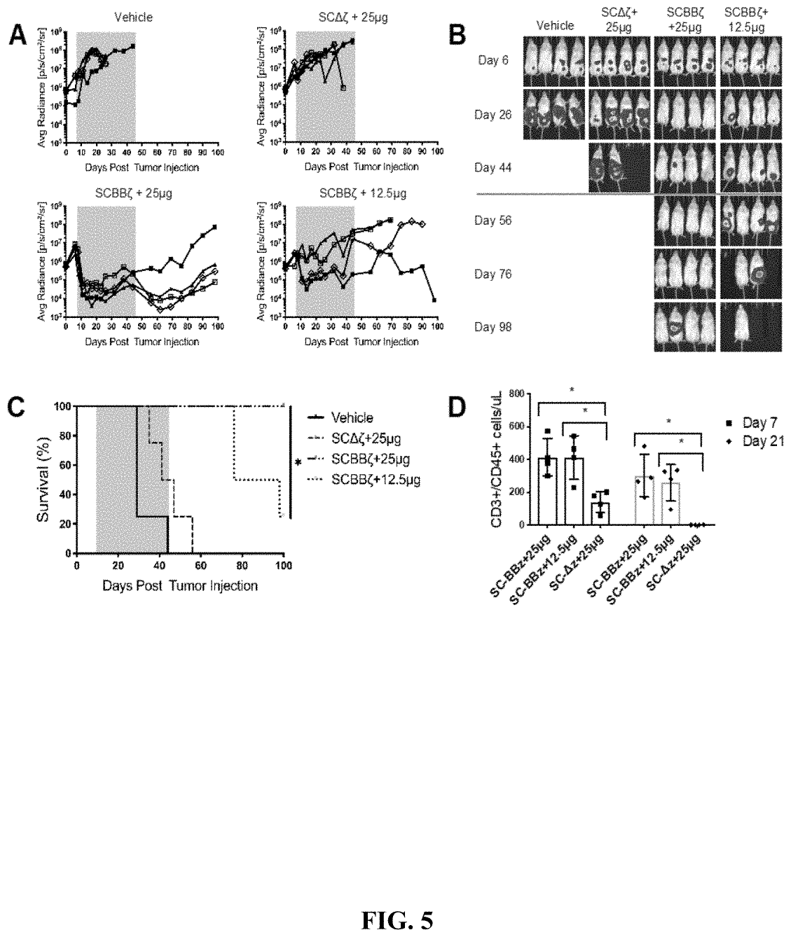

We tested the in vivo efficacy of SpyCatcher-BBζ T cells using an intraperitoneal model of ovarian cancer. Her2+SKOV3 ovarian cancer tumor cells were injected into the peritoneal cavity (I.P.) and allowed to establish for 7 days. On day 7, mice received I.P. injection of SpyCatcher-BBζ T cells armed with Herceptin-ST at a concentration of 1000 nM. One group was administered armed SpyCatcher-Δζ T cells to control for any tumor reduction caused by T cell infusion or targeting ligand administration independent of antigen dependent T cell stimulation. Beginning on day 8, additional targeting ligand was administered followed by continual dosing every 3 days during the dosing window (FIG. 5A; orange box).

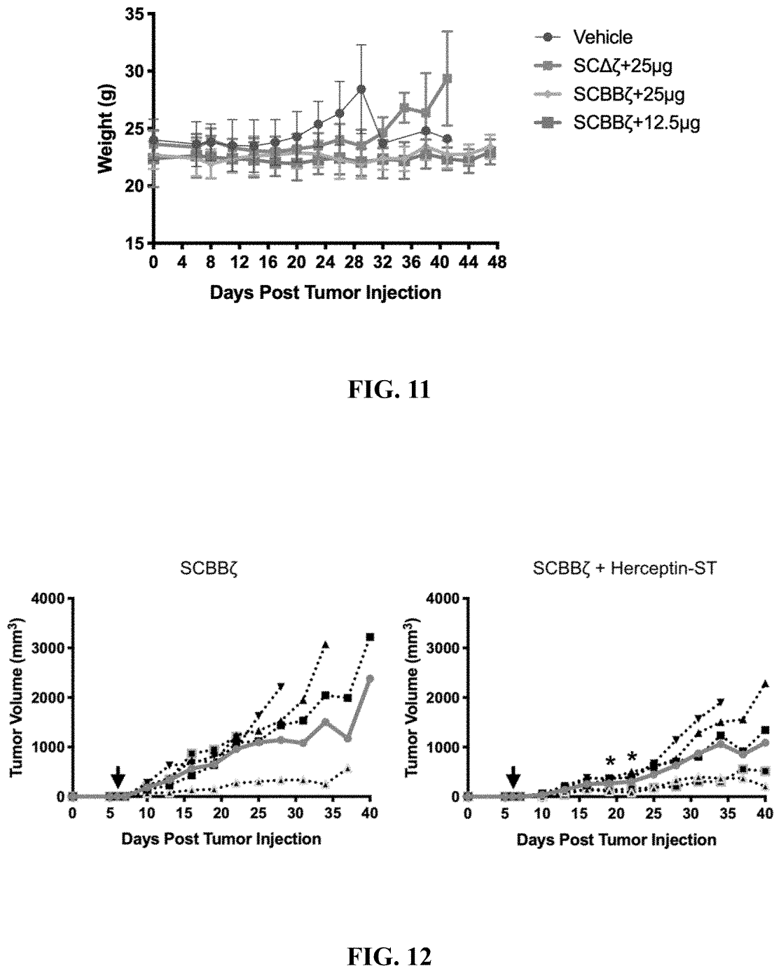

Treatment with SpyCatcher-Δζ T cells co-administered with a 25 μg dose of Herceptin-ST was similar to the vehicle control, demonstrating that the tumor was not sensitive to targeting ligand alone or infusion of signaling deficient T cells (FIGS. 5A-5B). SpyCatcher-BBζ T cells co-administered with a 25 μg dose of Herceptin-ST were able to clear detectable tumor in 3/4 mice, which resulted in prolonged survival relative to all other treatment groups (FIGS. 5A-5C). Tumor relapse was seen in one mouse beginning 30 days after cessation of targeting ligand treatment. SpyCatcher-BBζ T cells co-administered with a 12.5 μg dose of Herceptin-ST showed a transient reduction of tumor burden in some mice, but did not maintain efficacy over the course of treatment, demonstrating that sufficient levels of targeting ligand must be provided to drive tumor clearance. Peak peripheral blood T cell levels were detected on day 7 post T cell infusion in all groups, with signaling enabled SpyCatcher-BBζ T cell counts exceeding those in the SpyCatcher-Δζ group (FIG. 5D). Human T cell counts in SpyCatcher-BBζ T cell groups were similar between the two targeting ligand dose groups, implicating targeting ligand availability as one limitation to effective treatment. Additionally, weight loss due to toxicity was not seen in treatment groups, while weight gain was seen in control group mice due to ascites formation during tumor progression (FIG. 11). To assess systemic delivery of the SpyCatcher system, preclinical testing was performed in a rapidly growing, highly aggressive subcutaneous Her2+SKOV3 tumor model. Results showed that I.P. injected SpyCatcher-BBζ T cells co-administered with Herceptin-ST lead to reduction in tumor volume out to day 22 post tumor inoculation, compared to SpyCatcher-BBζ T cells alone (FIG. 12).

Universal immune receptors are an emerging technology aimed at improving standard CAR T cell therapies and addressing limitations in therapeutic design (Minutolo et al., Frontiers in Oncology 2019, 9, 176). Through the use of a targeting ligand to redirect T cells against antigen expressing tumor cells, UIRs allow for dose-dependent control of T cell effector function, while also enabling the use of a single receptor, single cell product to target multiple tumor antigens. Current UIR platforms rely on noncovalent interactions between their extracellular adapter protein and targeting ligand tag, rendering them unable to be covalently loaded with antigen specificity prior to infusion.

Here, a novel universal immune receptor platform was developed that allows for post-translational covalent attachment of targeting ligands to an immune receptor via SpyCatcher/SpyTag chemistry, mediating the redirection of T cells against multiple tumor associated antigens. Two functional SpyCatcher immune receptor constructs containing extracellular SpyCatcher domains and either CD28-CD3ζ or 4-1BB-CD3ζ intracellular domains were generated, and their expression in primary human T cells was demonstrated. To redirect SpyCatcher immune receptor expressing T cells against target tumor antigens, we used two methods of targeting ligand production. Using the LASIC adaptor protein method developed by Hui et. al, we were able to site-specifically label off-the-shelf clinical grade antibodies with SpyTag moieties (Hui et al., Bioconjugate Chemistry 2015, 26 (8), 1456-1460). This methodology allows for a potentially advantageous pairing of SpyCatcher T cells and clinical grade antibodies in patients who do not benefit from or become resistant to antibody monotherapy alone, if the resistance in not due to antigen loss (Rezvani et al., Best Practice & Research Clinical Haematology 2011, 24 (2), 203-216; Karapetis et al., The New England Journal of Medicine 2008, 359 (17), 1757-1765). Monovalent targeting ligands were further generated through genetic fusion of SpyTag to various designed ankyrin repeat proteins (DARPins). Through the use of these targeting ligands, the capability of the SpyCatcher immune receptor to target multiple tumor antigens in vitro was demonstrated. Transfer of SpyCatcher T cells along with SpyTag targeting ligands lead to control of outgrowth in aggressive subcutaneous tumors and clearance of established intraperitoneal disease in immunodeficient mouse xenograft models.

Notably, the formation of a covalent bond between SpyCatcher and SpyTag was critical for optimal T cell effector function. The transient bond formed between the SpyTag-DA mutant and SpyCatcher receptor (Kd=200 nM) led to a substantial loss of receptor loading (FIG. 2D), and this in turn resulted in decreased lytic capacity (FIG. 3G). In instances where the targeting ligand was added directly to culture, the inability to form a covalent bond greatly diminished or completely abolished effector function at low concentrations (FIG. 3F). This indicates that covalent bond formation between the targeting ligand and the receptor may be important in instances where targeting ligand concentration are low.

It is disclosed herein that the SpyCatcher immune receptor system is capable of addressing some of the limitations currently facing CAR T cell therapy. One of these limitations is the inability to control CAR T cells post-infusion. Upon binding to target antigen, CAR T cell activation and proliferation can occur at a rapid rate, and can lead to issues such as CRS or on-target, off-tumor lysis of normal tissue (Grupp et al., The New England Journal of Medicine 2013,368 (16), 1509-1518; Morgan et al., Molecular Therapy 2010, 18 (4), 843-851; Lamers et al., Molecular Therapy 2013, 21 (4), 904-912). It was shown that the effector function of SpyCatcher T cells is titratable based on the amount of targeting ligand either covalently loaded onto the receptor prior to tumor exposure or injected post-infusion, and that SpyCatcher T cells without targeting ligand are unable to target tumor cells. It was seen that suboptimal doses of targeting ligand lead to tumor outgrowth in our I.P. mouse model, indicating that continued administration of targeting ligand at sufficient levels is necessary for prolonged T cell function. The ability to escalate, decrease, or withdraw targeting ligand dose as a means of attenuating T cell effector function with the SpyCatcher system could allow for the mitigation of on-target, off-tumor toxicity. SpyCatcher T cells function with the use of antibodies, as well as DARPins, and have the potential to be expanded further to the use of scFvs, Fabs, and small molecule conjugates. This could allow for another potential layer of control, as targeting ligands with short half-lives would more lead to more rapid cessation of SpyCatcher T cell effector function. Patients who receive CD19 targeted CAR T cell therapy sustain long term B cell aplasia due to continual elimination of CD19+ normal B cells. SpyCatcher T cells are unreactive in the absence of targeting ligand, and therefore could allow for re-establishment of a normal B cell population after tumor clearance via discontinuation of targeting ligand.

Outgrowth of an antigen negative tumor population is another issue facing single antigen targeting T cell therapy. This can occur for a multitude of reasons, which include heterogeneous tumor antigen expression, down regulation of tumor antigen, trogocytosis, or expression of splice variants (Bagashev et al., The Importance of CD19 Exon 2 for Surface Localization: Closing the Ig-like Loop. 2015; Sotillo et al., Cancer Discovery 2015, 5 (12), 1282-1295; Jacoby et al., Nature Communications 2016, 7 (1), ncomms12320; Gardner et al., Blood 2016, 127 (20), 2406-2410; Hamieh et al., Nature 2019, 1-5; Ruella et al. Nature Medicine 2018, 24 (10), 1499-1503). To overcome tumor escape via antigen loss other groups have proposed to express multiple CARs in a single T cell product 57, express CARs with multiple epitope binding domains (Grada et al., Molecular Therapy—Nucleic Acids 2013, 2 (7), e105; Hegde et al., J Clin Invest 2016, 126 (8), 3036-3052; Schneider et al., J Immunother Cancer 2017, 5 (1), 42), or make a combined T cell product containing two distinct CAR populations (Ruella et al., J Clin Invest 2016, 126 (10), 3814-3826). While these approaches may help mitigate antigen loss, they do not address safety and toxicity concerns, and still limit the total number of targetable antigens. SpyCatcher T cells are capable of lysing a broad range or tumor antigens, with proof of principle established here for Her2, EGFR, EpCAM, and CD20. The ability to arm SpyCatcher immune receptor T cells with multiple targeting ligands simultaneously, allowing for the creation of a single vector and single cell product capable of targeting multiple tumor antigens simultaneously, was further demonstrated. The turnover of armed receptor coupled with the continual expression of new SpyCatcher receptor by the T cells enables re-arming with targeting ligand (FIG. 3E) and the potential for sequential antigen targeting, as was previously demonstrated with a biotin binding immune receptor (Urbanska et al., Cancer Research 2012, 72 (7), 1844-1852).

Though the SpyCatcher immune receptor system has shown promise both in vitro and in vivo, it is important to acknowledge the potential limitations to its expanded use. The SpyCatcher/SpyTag system itself was derived from the second immunoglobulin-like collagen adhesin domain (CnaB2) of the Streptococcus pyogenes fibronectin-binding protein FbaB. The bacterial origin of these proteins means that they are potentially immunogenic, and therefore may be suppressed by the patient's immune system. A similar issue has plagued CARs with murine derived scFv domains, against which patients develop human anti-mouse antibodies (HAMA) (Beatty et al., Cancer Immunology Research 2014, 2 (2), 112-120). However, previous studies have tested SpyCatcher immunogenicity in immune competent mice and have reported that truncated versions of the SpyCatcher protein, similar to that used here, induced significantly lower antibody levels (Liu et al., Sci Rep 2014, 4, 7266).

The SpyCatcher immune receptor, and UIRs in general, present an exciting new avenue for therapeutic investigation. However, much like in the field of CAR T cell therapy, optimal clinical translation will rely on facing unique challenges related to UIRs. For instance, the ability to target multiple antigens with a single receptor relies upon the discovery, validation, and safety profiling of tumor associated antigens and clinical-grade targeting ligands against those antigens. For targeting some antigens, there may also be challenges in designing optimal universal immune receptor and their targeting ligand pairs, since optimal targeting ligand size and receptor hinge domain spacing can vary based on the targeted antigenic epitope (Rodgers et al., Proc. Natl. Acad. Sci. U.S.A. 2016, 113 (4); Ma et al., Proc. Natl. Acad. Sci. U.S.A. 2016, 113 (4), E450-E458). In addition, the clinical production of UIR T cells will often rely upon patient T cells as the starting source cell material, akin to CAR T cell therapy. Accordingly, UIR therapies will also need to address the shared issues of pre-existent T cell exhaustion and senescence, poor proliferation and persistence after infusion and the lack of T cell infiltration into solid tumors (Maude et al., The New England Journal of Medicine 2014, 371 (16), 1507-1517; Fraietta et al., Nature Medicine 2018, 24 (5), 563-571; Lanitis et al., Ann Oncol 2017; Mueller et al., Clinical Cancer Research 2018, clincanres. 0758.2018, that can limit otherwise effective CART cells therapy in certain indications.

The SpyCatcher immune receptor described herein is the first universal immune receptor to allow for post-translational covalent loading of targeting ligands for subsequent redirection of T cells against an array of tumor antigens in vitro and in vivo. This platform technology provides a method for single vector, single receptor targeting of multiple antigens simultaneously, while also allowing for continual re-arming to generate, regulate, and diversify a sustained T cell response over time.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### Enumerated Embodiments

The following enumerated embodiments are provided, the numbering of which is not to be construed as designating levels of importance.

Embodiment 1 provides a method for stimulating a universal immune receptor-mediated immune response to a tumor in a mammal, wherein the tumor co-expresses at least two different antigens, the method comprising (a) administering to the mammal a cell genetically modified to express an immune receptor comprising a T cell receptor intracellular signaling domain, a transmembrane domain, and an extracellular domain comprising an adaptor molecule, (b) administering to the mammal a first agent linked to a reciprocal adaptor molecule, wherein the first agent specifically binds a first antigen expressed by the tumor, and (c) administering to the mammal a second agent linked to a reciprocal adaptor molecule, wherein the second agent specifically binds a second antigen expressed by the tumor and wherein the first antigen and the second antigen are different antigens.

Embodiment 2 provides a method of treating a cancer in a mammal in need thereof, wherein the cancer co-expresses at least two different antigens, the method comprising (a) administering to the mammal an effective amount of a cell genetically modified to express an immune receptor comprising a T cell receptor intracellular signaling domain, a transmembrane domain, and an extracellular domain comprising an adaptor molecule, (b) administering to the mammal an effective amount of a first agent linked to a reciprocal adaptor molecule, wherein the first agent specifically binds a first antigen expressed by the cancer, and (c) administering to the mammal an effective amount of a second agent linked to a reciprocal adaptor molecule, wherein the second agent specifically binds a second antigen expressed by the cancer and wherein the first antigen and the second antigen are different antigens.

Embodiment 3 provides the method of embodiment 1 or 2, wherein the extracellular domain comprises a SpyCatcher or SpyTag.

Embodiment 4 provides the method of any one of embodiments 1-3, wherein the first and/or second agent is linked to a SpyTag or SpyCatcher.

Embodiment 5 provides the method of any one of embodiments 1-4, wherein the first and/or second agent is an antibody, an antibody fragment, a scFv, or a DARPin.

Embodiment 6 provides the method of any one of embodiments 1-5, wherein the first and/or second agent is an antibody and is human IgG.

Embodiment 7 provides the method of any one of embodiments 1-6, wherein the reciprocal adaptor molecule, SpyTag, or SpyCatcher is linked to the first and/or second agent via light activated site-specific conjugation (LASIC).

Embodiment 8 provides the method of any one of embodiments 1-7, wherein the cell is a T cell, a Natural Killer (NK) cell, a cytotoxic T lymphocyte (CTL), a macrophage, a stem cell, or a regulatory T cell.

Embodiment 9 provides the method of any one of embodiments 1-8, wherein the cell is an autologous cell.

Embodiment 10 provides the method of any one of embodiments 1-9, wherein the immune receptor further comprises an intracellular domain of a costimulatory molecule.

Embodiment 11 provides the method of embodiment 10, wherein the costimulatory molecule is 4-1BB, CD28, CD27, CD2, CD3, OX40, CD30, CD40, PD-1, ICOS, lymphocyte function-associated antigen-1 (LFA-1), CD7, LIGHT, NKG2C, B7-H3, or a ligand that specifically binds with CD83.

Embodiment 12 provides the method of any one of embodiments 1-11, wherein the cell is contacted with the first and/or second agent prior to administration to the mammal to generate a pre-armed cell and the pre-armed cell is subsequently administered to the mammal.

Embodiment 13 provides the method of any one of embodiments 1-11, wherein the cell is administered to the mammal prior to administering the first and/or second agent to the mammal.

Embodiment 14 provides a method of generating a level of lytic activity against a tumor, the method comprising (a) contacting an amount of cells with an amount of an agent linked to a reciprocal adaptor molecule, wherein the cells are genetically modified to express an immune receptor comprising a T cell receptor intracellular signaling domain, a transmembrane domain, and an extracellular domain wherein the extracellular domain comprises an adaptor molecule, wherein the agent specifically binds an antigen expressed by the tumor, and wherein the amount of the agent and/or the amount of cells is selected to generate the level of lytic activity against the tumor.

Embodiment 15 provides a method of treating a cancer in a mammal in need thereof, the method comprising (a) administering to the mammal an amount of cells genetically modified to express an immune receptor comprising a T cell receptor intracellular signaling domain, a transmembrane domain, and an extracellular domain comprising an adaptor molecule, and (b) administering to the mammal an amount of an agent linked to a reciprocal adaptor molecule, wherein the agent specifically binds an antigen expressed by the cancer, wherein the amount of the cells and/or the amount of the agent is selected to provide a level of lytic activity against the cancer.

Embodiment 16 provides the method of any one of embodiments 14-15, wherein increasing the amount of agent relative to the amount of the cells increases the level of lytic activity and decreasing the amount of agent relative to the amount of the cells decreases the level of lytic activity.

Embodiment 17 provides the method of any one of embodiments 14-16, wherein the extracellular domain comprises a SpyCatcher or SpyTag.

Embodiment 18 provides the method of any one of embodiments 14-17, wherein the agent is linked to a SpyTag or SpyCatcher.

Embodiment 19 provides the method of any one of embodiments 14-18, wherein the agent is an antibody, an antibody fragment, a scFv, or a DARPin.

Embodiment 20 provides the method of any one of embodiments 14-19, wherein the agent is an antibody and is human IgG.

Embodiment 21 provides the method of any one of embodiments 14-20, wherein the reciprocal adaptor molecule, SpyTag or SpyCatcher is linked to the agent via light activated site-specific conjugation (LASIC).

Embodiment 22 provides the method of any one of embodiments 14-21, wherein the cell is a T cell, a Natural Killer (NK) cell, a cytotoxic T lymphocyte (CTL), a macrophage, a stem cell, or a regulatory T cell.

Embodiment 23 provides the method of any one of embodiments 14-22, wherein the cell is an autologous cell.

Embodiment 24 provides the method of any one of embodiments 14-23, wherein the immune receptor further comprises an intracellular domain of a costimulatory molecule.

Embodiment 25 provides the method of any one of embodiments 14-24, wherein the costimulatory molecule is 4-1BB, CD28, CD27, CD2, CD3, OX40, CD30, CD40, PD-1, ICOS, lymphocyte function-associated antigen-1 (LFA-1), CD7, LIGHT, NKG2C, B7-H3, or a ligand that specifically binds with CD83.

Embodiment 26 provides the method of any one of embodiments 15-25, wherein the cell is contacted with the agent prior to administration to the mammal to generate a pre-armed cell and the pre-armed cell is subsequently administered to the mammal.

Embodiment 27 provides the method of any one of embodiments 15-26, wherein the cell is administered to the mammal prior to administering the agent to the mammal.

Embodiment 28 provides a method of stimulating a universal immune receptor-mediated immune response to a tumor in a mammal in need thereof, the method comprising (a) administering to the mammal a cell genetically modified to express an immune receptor comprising a T cell receptor intracellular signaling domain, a transmembrane domain, and an extracellular domain comprising an adaptor molecule, and (b) subsequently administering to the mammal an agent linked to a reciprocal adaptor molecule, wherein the agent specifically binds an antigen expressed by the tumor.

Embodiment 29 provides a method of treating a cancer in a mammal in need thereof, the method comprising (a) administering to the mammal an effective amount of a T cell genetically modified to express an immune receptor comprising a T cell receptor intracellular signaling domain, a transmembrane domain, and an extracellular domain comprising an adaptor molecule, and (b) subsequently administering to the mammal an effective amount of an agent linked to a reciprocal adaptor molecule, wherein the agent specifically binds an antigen expressed by the cancer.

Embodiment 30 provides the method of any one of embodiments 28 or 29, wherein the extracellular domain comprises a SpyCatcher or SpyTag.

Embodiment 31 provides the method of any one of embodiments 28-30, wherein the agent is linked to a SpyTag or SpyCatcher.

Embodiment 32 provides the method of any one of embodiments 28-31, wherein the agent is an antibody, an antibody fragment, a scFv, or a DARPin.

Embodiment 33 provides the method of any one of embodiments 28-32, wherein the agent is an antibody and is human IgG.

Embodiment 34 provides the method of any one of embodiments 28-33, wherein the reciprocal adaptor molecule, SpyTag or SpyCatcher is linked to the agent via light activated site-specific conjugation (LASIC).

Embodiment 35 provides the method of any one of embodiments 28-34, wherein the cell is a T cell, a Natural Killer (NK) cell, a cytotoxic T lymphocyte (CTL), a macrophage, a stem cell, or a regulatory T cell.

Embodiment 36 provides the method of any one of embodiments 28-35, wherein the cell is an autologous cell.

Embodiment 37 provides the method of any one of embodiments 28-36, wherein the immune receptor further comprises an intracellular domain of a costimulatory molecule.

Embodiment 38 provides the method of any one of embodiments 28-37, wherein the costimulatory molecule is 4-1BB, CD28, CD27, CD2, CD3, OX40, CD30, CD40, PD-1, ICOS, lymphocyte function-associated antigen-1 (LFA-1), CD7, LIGHT, NKG2C, B7-H3, or a ligand that specifically binds with CD83.

Embodiment 39 provides a method of quantifying turnover of a universal immune receptor on a cell surface, the method comprising (a) contacting a cell genetically modified to express an immune receptor comprising a T cell receptor intracellular signaling domain, a transmembrane domain, and an extracellular domain comprising an adapter molecule with an agent linked to a reciprocal adapter molecule, thereby generating an armed receptor; and (b) determining an amount of the armed receptor relative to a reference amount.

Embodiment 40 provides the method of embodiment 39, wherein the reference amount is an amount of the armed receptor at a prior time.

Embodiment 41 provides the method of embodiment 40, wherein the amount of the armed receptor is determined by labeling the agent and detecting the labeled agent.

Embodiment 42 provides the method of embodiment 41, wherein the agent is labeled by linking or contacting the agent with a labeling molecule comprising a myc-tag, FLAG-tag, His-tag, HA-tag, a fluorescent protein (e.g., a green fluorescent protein (GFP)), a fluorophore (e.g., tetramethylrhodamine (TRITC)), fluorescein isothiocyanate (FITC), dinitrophenol, peridin chlorophyll protein complex, phycoerythrin (PE), histidine, biotin, streptavidin, avidin, horse radish peroxidase, palmitoylation, nitrosylation, alkalanine phosphatase, glucose oxidase, Glutathione S-transferase (GST), maltose binding protein, a radioisotope, and any types of compounds used for radioisotope labeling including, 1,4,7,10-tetraazacyclododecane-1,4,7,10-tetraacetic acid (DOTA), diethylene triamine pentaacetic acid (DTPA), 1,4,7-triazacyclononane-1,4,7-triacetic acid (NOTA), or allophycocyanin (APC).

Embodiment 43 provides the method of any one of embodiments 39-42, further comprising the step of (c) contacting the cell and the agent with a tumor cell.

Embodiment 44 provides the method of any one of embodiments 39-43, wherein the extracellular domain comprises a SpyCatcher.

Embodiment 45 provides the method of any one of embodiments 39-44, wherein the agent is linked to a SpyTag.

Embodiment 46 provides the method of any one of embodiments 39-45, wherein the agent is an antibody, an antibody fragment, a scFv, or a DARPin.

Embodiment 47 provides the method of any one of embodiments 39-46, wherein the agent is an antibody and is human IgG.

Embodiment 48 provides the method of any one of embodiments 39-47, wherein the cell is a T cell, a Natural Killer (NK) cell, a cytotoxic T lymphocyte (CTL), a macrophage, a stem cell, or a regulatory T cell.

Embodiment 49 provides the method of any one of embodiments 39-48, wherein the immune receptor further comprises an intracellular domain of a costimulatory molecule.

Embodiment 50 provides the method of any one of embodiments 39-49, wherein the costimulatory molecule is 4-1BB, CD28, CD27, CD2, CD3, OX40, CD30, CD40, PD-1, ICOS, lymphocyte function-associated antigen-1 (LFA-1), CD7, LIGHT, NKG2C, B7-H3, or a ligand that specifically binds with CD83.

Embodiment 51 provides a method of stimulating a universal immune receptor-mediated immune response to a tumor in a mammal in need thereof, the method comprising (a) administering to the mammal an effective amount of a cell genetically modified to express an immune receptor comprising a T cell receptor intracellular signaling domain, an intracellular domain of 4-1BB, a transmembrane domain, and an extracellular domain comprising an adaptor molecule, and (b) administering to the mammal an effective amount of an agent linked to a reciprocal adaptor molecule, wherein the agent specifically binds an antigen expressed by the tumor, wherein the tumor is pre-determined to express the antigen at an increased level relative to a reference level.

Embodiment 52 provides a method of treating a cancer in a mammal in need thereof, the method comprising (a) administering to the mammal an amount of a cell genetically modified to express an immune receptor comprising a T cell receptor intracellular signaling domain, an intracellular domain of 4-1BB, a transmembrane domain, and an extracellular domain comprising an adaptor molecule, and (b) administering to the mammal an amount of an agent linked to a reciprocal adaptor molecule, wherein the agent specifically binds an antigen expressed by the cancer, wherein the cancer is pre-determined to express the antigen at an increased level relative to a reference level.

Embodiment 53 provides the method of any one of embodiments 51 or 52, wherein the reference level is a level of expression of the antigen on healthy tissue.

Embodiment 54 provides the method of any one of embodiments 51-53, wherein the extracellular domain comprises a SpyCatcher or SpyTag.

Embodiment 55 provides the method of any one of embodiments 51-54, wherein the agent is linked to a SpyTag or SpyCatcher.

Embodiment 56 provides the method of any one of embodiments 51-55, wherein the agent is an antibody, an antibody fragment, a scFv, or a DARPin.

Embodiment 57 provides the method of any one of embodiments 51-56, wherein the agent is an antibody and is human IgG.

Embodiment 58 provides the method of any one of embodiments 51-57, wherein the SpyTag or SpyCatcher is linked to the agent via light activated site-specific conjugation (LASIC).

Embodiment 59 provides the method of any one of embodiments 51-58, wherein the cell is a T cell, a Natural Killer (NK) cell, a cytotoxic T lymphocyte (CTL), a macrophage, a stem cell, or a regulatory T cell.

Embodiment 60 provides the method of any one of embodiments 51-59, wherein the cell is an autologous cell.

Embodiment 61 provides the method of any one of embodiments 51-60, wherein the cell is contacted with agent prior to administration to the mammal to generate a pre-armed cell and the pre-armed cell is subsequently administered to the mammal.

Embodiment 62 provides the method of any one of embodiments 51-61, wherein the cell is administered to the mammal prior to administering the agent to the mammal.

Embodiment 63 provides a genetically modified cell, a first agent, and a second agent, for use in stimulating a universal immune receptor-mediated immune response to a tumor in a mammal in need thereof, wherein the tumor co-expresses at least two different antigens, wherein the cell is genetically modified to express an immune receptor comprising a T cell receptor intracellular signaling domain, a transmembrane domain, and an extracellular domain comprising an adaptor molecule, wherein the first agent is linked to a reciprocal adaptor molecule, and wherein the first agent specifically binds a first antigen expressed by the tumor; wherein the second agent is linked to a reciprocal adaptor molecule, and wherein the second agent specifically binds a second antigen expressed by the tumor; wherein the first antigen and the second antigen are different antigens, and wherein the immune receptor, first agent, and second agent are administered to the mammal.

Embodiment 64 provides a genetically modified cell, a first agent, and a second agent, for use in treating cancer in a mammal in need thereof, wherein the cancer co-expresses at least two different antigens, wherein the cell is genetically modified to express an immune receptor comprising a T cell receptor intracellular signaling domain, a transmembrane domain, and an extracellular domain comprising an adaptor molecule, wherein the first agent is linked to a reciprocal adaptor molecule, and wherein the first agent specifically binds a first antigen expressed by the cancer; wherein the second agent is linked to a reciprocal adaptor molecule, and wherein the second agent specifically binds a second antigen expressed by the cancer; and wherein the first antigen and the second antigen are different antigens.

Embodiment 65 provides a genetically modified cell and an agent for use in generating a level of lytic activity against a tumor, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds a an antigen expressed by the tumor; wherein an amount of the cell is contacted with an amount of the agent and wherein the amount of the agent relative to the amount of cells is selected to generate the level of lytic activity against the tumor.

Embodiment 66 provides a genetically modified cell and an agent for use in treating cancer in a mammal in need thereof, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds an antigen expressed by the cancer; wherein an amount of the cell and an amount of the agent is administered to the mammal, wherein the amount of the cell and/or the amount of the agent is selected to provide a level of lytic activity against the cancer.

Embodiment 67 provides a genetically modified cell and an agent for use in stimulating a universal immune receptor-mediated immune response to a tumor in a mammal in need thereof, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds a an antigen expressed by the tumor; wherein an amount of the cell is administered to the mammal and an amount of the agent is subsequently administered to the mammal.

Embodiment 68 provides a genetically modified cell and an agent for use in treating cancer in a mammal in need thereof, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds an antigen expressed by the cancer; wherein an amount of the cell is administered to the mammal and an amount of the agent is subsequently administered to the mammal.

Embodiment 69 provides a genetically modified cell and an agent for use in stimulating a universal immune receptor-mediated immune response to a tumor in a mammal in need thereof, wherein the cell is genetically modified to express an immune receptor comprising a T cell receptor intracellular signaling domain, an intracellular domain of 4-1BB, a transmembrane domain, and an extracellular domain comprising an adaptor molecule, wherein the agent is linked to a reciprocal adaptor molecule, and wherein the agent specifically binds a an antigen expressed by the tumor; wherein an effective amount of the cell is administered to the mammal and an effective amount of the agent is administered to the mammal, and wherein the tumor is pre-determined to express the antigen at an increased level relative to a reference level.

Embodiment 70 provides a genetically modified cell and an agent for use in treating a cancer in a mammal in need thereof, wherein the cell is genetically modified to express an immune receptor comprising a T cell receptor intracellular signaling domain, an intracellular domain of 4-1BB, a transmembrane domain, and an extracellular domain comprising an adaptor molecule, wherein the agent is linked to a reciprocal adaptor molecule, and wherein the agent specifically binds a an antigen expressed by the tumor; wherein an effective amount of the cell is administered to the mammal and an effective amount of the agent is administered to the mammal, and wherein the tumor is pre-determined to express the antigen at an increased level relative to a reference level.

Embodiment 71 provides use of a genetically modified cell, a first agent, and a second agent, in stimulating a universal immune receptor-mediated immune response to a tumor in a mammal in need thereof, wherein the tumor co-expresses at least two different antigens, wherein the cell is genetically modified to express an immune receptor comprising a T cell receptor intracellular signaling domain, a transmembrane domain, and an extracellular domain comprising an adaptor molecule, wherein the first agent is linked to a reciprocal adaptor molecule, and wherein the first agent specifically binds a first antigen expressed by the tumor; wherein the second agent is linked to a reciprocal adaptor molecule, and wherein the second agent specifically binds a second antigen expressed by the tumor; wherein the first antigen and the second antigen are different antigens, and wherein the immune receptor, first agent, and second agent are administered to the mammal.

Embodiment 72 provides use of a genetically modified cell, a first agent, and a second agent, in the treatment of cancer in a mammal in need thereof, wherein the cancer co-expresses at least two different antigens, wherein the cell is genetically modified to express an immune receptor comprising a T cell receptor intracellular signaling domain, a transmembrane domain, and an extracellular domain comprising an adaptor molecule, wherein the first agent is linked to a reciprocal adaptor molecule, and wherein the first agent specifically binds a first antigen expressed by the cancer; wherein the second agent is linked to a reciprocal adaptor molecule, and wherein the second agent specifically binds a second antigen expressed by the cancer; and wherein the first antigen and the second antigen are different antigens.

Embodiment 73 provides use of a genetically modified cell and an agent in generating a level of lytic activity against a tumor, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds a an antigen expressed by the tumor; wherein an amount of the cell is contacted with an amount of the agent and wherein the amount of the agent relative to the amount of cells is selected to generate the level of lytic activity against the tumor.

Embodiment 74 provides use of a genetically modified cell and an agent in the treatment of cancer in a mammal in need thereof, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds an antigen expressed by the cancer; wherein an amount of the cell and an amount of the agent is administered to the mammal, wherein the amount of the cell and/or the amount of the agent is selected to provide a level of lytic activity against the cancer.

Embodiment 75 provides use of a genetically modified cell and an agent in stimulating universal immune receptor-mediated immune response to a tumor in a mammal in need thereof, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds a an antigen expressed by the tumor; wherein an amount of the cell is administered to the mammal and an amount of the agent is subsequently administered to the mammal.

Embodiment 76 provides use of a genetically modified cell and an in the treatment of cancer in a mammal in need thereof, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds an antigen expressed by the cancer; wherein an amount of the cell is administered to the mammal and an amount of the agent is subsequently administered to the mammal.

Embodiment 77 provides use of a genetically modified cell and an agent in stimulating a universal immune receptor-mediated immune response to a tumor in a mammal in need thereof, wherein the cell is genetically modified to express an immune receptor comprising a T cell receptor intracellular signaling domain, an intracellular domain of 4-1BB, a transmembrane domain, and an extracellular domain comprising an adaptor molecule, wherein the agent is linked to a reciprocal adaptor molecule, and wherein the agent specifically binds a an antigen expressed by the tumor; wherein an effective amount of the cell is administered to the mammal and an effective amount of the agent is administered to the mammal, and wherein the tumor is pre-determined to express the antigen at an increased level relative to a reference level.

Embodiment 78 provides use of a genetically modified cell and an agent in the treatment of a cancer in a mammal in need thereof, wherein the cell is genetically modified to express an immune receptor comprising a T cell receptor intracellular signaling domain, an intracellular domain of 4-1BB, a transmembrane domain, and an extracellular domain comprising an adaptor molecule, wherein the agent is linked to a reciprocal adaptor molecule, and wherein the agent specifically binds a an antigen expressed by the tumor; wherein an effective amount of the cell is administered to the mammal and an effective amount of the agent is administered to the mammal, and wherein the tumor is pre-determined to express the antigen at an increased level relative to a reference level.

1. A method for stimulating a universal immune receptor-mediated immune response to a tumor in a mammal, wherein the tumor co-expresses at least two different antigens, the method comprising
(a) administering to the mammal a cell genetically modified to express an immune receptor comprising a T cell receptor intracellular signaling domain, a transmembrane domain, and an extracellular domain comprising an adaptor molecule,
(b) administering to the mammal a first agent linked to a reciprocal adaptor molecule, wherein the first agent specifically binds a first antigen expressed by the tumor, and
(c) administering to the mammal a second agent linked to a reciprocal adaptor molecule, wherein the second agent specifically binds a second antigen expressed by the tumor and wherein the first antigen and the second antigen are different antigens.

2. A method of treating a cancer in a mammal in need thereof, wherein the cancer co-expresses at least two different antigens, the method comprising
(a) administering to the mammal an effective amount of a cell genetically modified to express an immune receptor comprising a T cell receptor intracellular signaling domain, a transmembrane domain, and an extracellular domain comprising an adaptor molecule,
(b) administering to the mammal an effective amount of a first agent linked to a reciprocal adaptor molecule, wherein the first agent specifically binds a first antigen expressed by the cancer, and
(c) administering to the mammal an effective amount of a second agent linked to a reciprocal adaptor molecule, wherein the second agent specifically binds a second antigen expressed by the cancer and wherein the first antigen and the second antigen are different antigens.

3. The method of claim 1 or 2, wherein the extracellular domain comprises a SpyCatcher or SpyTag.

4. The method of any one of claims 1-3, wherein the first and/or second agent is linked to a SpyTag or SpyCatcher.

5. The method of any one of claims 1-4, wherein the first and/or second agent is an antibody, an antibody fragment, a scFv, or a DARPin.

6. The method of any one of claims 1-5, wherein the first and/or second agent is an antibody and is human IgG.

7. The method of any one of claims 1-6, wherein the reciprocal adaptor molecule, SpyTag, or SpyCatcher is linked to the first and/or second agent via light activated site-specific conjugation (LASIC).

8. The method of any one of claims 1-7, wherein the cell is a T cell, a Natural Killer (NK) cell, a cytotoxic T lymphocyte (CTL), a macrophage, a stem cell, or a regulatory T cell.

9. The method of any one of claims 1-8, wherein the cell is an autologous cell.

10. The method of any one of claims 1-9, wherein the immune receptor further comprises an intracellular domain of a costimulatory molecule.

11. The method of claim 10, wherein the costimulatory molecule is 4-1BB, CD28, CD27, CD2, CD3, OX40, CD30, CD40, PD-1, ICOS, lymphocyte function-associated antigen-1 (LFA-1), CD7, LIGHT, NKG2C, B7-H3, or a ligand that specifically binds with CD83.

12. The method of any one of claims 1-11, wherein the cell is contacted with the first and/or second agent prior to administration to the mammal to generate a pre-armed cell and the pre-armed cell is subsequently administered to the mammal.

13. The method of any one of claims 1-11, wherein the cell is administered to the mammal prior to administering the first and/or second agent to the mammal.

14. A method of generating a level of lytic activity against a tumor, the method comprising
(a) contacting an amount of cells with an amount of an agent linked to a reciprocal adaptor molecule, wherein the cells are genetically modified to express an immune receptor comprising a T cell receptor intracellular signaling domain, a transmembrane domain, and an extracellular domain wherein the extracellular domain comprises an adaptor molecule, wherein the agent specifically binds an antigen expressed by the tumor, and
wherein the amount of the agent and/or the amount of cells is selected to generate the level of lytic activity against the tumor.

15. A method of treating a cancer in a mammal in need thereof, the method comprising
(a) administering to the mammal an amount of cells genetically modified to express an immune receptor comprising a T cell receptor intracellular signaling domain, a transmembrane domain, and an extracellular domain comprising an adaptor molecule, and
(b) administering to the mammal an amount of an agent linked to a reciprocal adaptor molecule, wherein the agent specifically binds an antigen expressed by the cancer, wherein the amount of the cells and/or the amount of the agent is selected to provide a level of lytic activity against the cancer.

16. The method of any one of claims 14-15, wherein increasing the amount of agent relative to the amount of the cells increases the level of lytic activity and decreasing the amount of agent relative to the amount of the cells decreases the level of lytic activity.

17. The method of any one of claims 14-16, wherein the extracellular domain comprises a SpyCatcher or SpyTag.

18. The method of any one of claims 14-17, wherein the agent is linked to a SpyTag or SpyCatcher.

19. The method of any one of claims 14-18, wherein the agent is an antibody, an antibody fragment, a scFv, or a DARPin.

20. The method of any one of claims 14-19, wherein the agent is an antibody and is human IgG.

21. The method of any one of claims 14-20, wherein the reciprocal adaptor molecule, SpyTag or SpyCatcher is linked to the agent via light activated site-specific conjugation (LASIC).

22. The method of any one of claims 14-21, wherein the cell is a T cell, a Natural Killer (NK) cell, a cytotoxic T lymphocyte (CTL), a macrophage, a stem cell, or a regulatory T cell.

23. The method of any one of claims 14-22, wherein the cell is an autologous cell.

24. The method of any one of claims 14-23, wherein the immune receptor further comprises an intracellular domain of a costimulatory molecule.

25. The method of any one of claims 14-24, wherein the costimulatory molecule is 4-1BB, CD28, CD27, CD2, CD3, OX40, CD30, CD40, PD-1, ICOS, lymphocyte function-associated antigen-1 (LFA-1), CD7, LIGHT, NKG2C, B7-H3, or a ligand that specifically binds with CD83.

26. The method of any one of claims 15-25, wherein the cell is contacted with the agent prior to administration to the mammal to generate a pre-armed cell and the pre-armed cell is subsequently administered to the mammal.

27. The method of any one of claims 15-26, wherein the cell is administered to the mammal prior to administering the agent to the mammal.

28. A method of stimulating a universal immune receptor-mediated immune response to a tumor in a mammal in need thereof, the method comprising
(a) administering to the mammal a cell genetically modified to express an immune receptor comprising a T cell receptor intracellular signaling domain, a transmembrane domain, and an extracellular domain comprising an adaptor molecule, and
(b) subsequently administering to the mammal an agent linked to a reciprocal adaptor molecule, wherein the agent specifically binds an antigen expressed by the tumor.

29. A method of treating a cancer in a mammal in need thereof, the method comprising
(a) administering to the mammal an effective amount of a T cell genetically modified to express an immune receptor comprising a T cell receptor intracellular signaling domain, a transmembrane domain, and an extracellular domain comprising an adaptor molecule, and
(b) subsequently administering to the mammal an effective amount of an agent linked to a reciprocal adaptor molecule, wherein the agent specifically binds an antigen expressed by the cancer.

30. The method of claim 28 or 29, wherein the extracellular domain comprises a SpyCatcher or SpyTag.

31. The method of any one of claims 28-30, wherein the agent is linked to a SpyTag or SpyCatcher.

32. The method of any one of claims 28-31, wherein the agent is an antibody, an antibody fragment, a scFv, or a DARPin.

33. The method of any one of claims 28-32, wherein the agent is an antibody and is human IgG.

34. The method of any one of claims 28-33, wherein the reciprocal adaptor molecule, SpyTag or SpyCatcher is linked to the agent via light activated site-specific conjugation (LASIC).

35. The method of any one of claims 28-34, wherein the cell is a T cell, a Natural Killer (NK) cell, a cytotoxic T lymphocyte (CTL), a macrophage, a stem cell, or a regulatory T cell.

36. The method of any one of claims 28-35, wherein the cell is an autologous cell.

37. The method of any one of claims 28-36, wherein the immune receptor further comprises an intracellular domain of a costimulatory molecule.

38. The method of any one of claims 28-37, wherein the costimulatory molecule is 4-1BB, CD28, CD27, CD2, CD3, OX40, CD30, CD40, PD-1, ICOS, lymphocyte function-associated antigen-1 (LFA-1), CD7, LIGHT, NKG2C, B7-H3, or a ligand that specifically binds with CD83.

39. A method of quantifying turnover of a universal immune receptor on a cell surface, the method comprising
(a) contacting a cell genetically modified to express an immune receptor comprising a T cell receptor intracellular signaling domain, a transmembrane domain, and an extracellular domain comprising an adapter molecule with an agent linked to a reciprocal adapter molecule, thereby generating an armed receptor; and
(b) determining an amount of the armed receptor relative to a reference amount.

40. The method of claim 39, wherein the reference amount is an amount of the armed receptor at a prior time.

41. The method of claim 40, wherein the amount of the armed receptor is determined by labeling the agent and detecting the labeled agent.

42. The method of claim 41, wherein the agent is labeled by linking or contacting the agent with a labeling molecule comprising a myc-tag, FLAG-tag, His-tag, HA-tag, a fluorescent protein (e.g., a green fluorescent protein (GFP)), a fluorophore (e.g., tetramethylrhodamine (TRITC)), fluorescein isothiocyanate (FITC), dinitrophenol, peridin chlorophyll protein complex, phycoerythrin (PE), histidine, biotin, streptavidin, avidin, horse radish peroxidase, palmitoylation, nitrosylation, alkalanine phosphatase, glucose oxidase, Glutathione S-transferase (GST), maltose binding protein, a radioisotope, a heavy metal, a supramagnetic nanoparticle, and any types of compounds used for radioisotope labeling including, 1,4,7,10-tetraazacyclododecane-1,4,7,10-tetraacetic acid (DOTA), diethylene triamine pentaacetic acid (DTPA), 1,4,7-triazacyclononane-1,4,7-triacetic acid (NOTA), or allophycocyanin (APC).

43. The method of any one of claims 39-42, further comprising the step of (c) contacting the cell and the agent with a tumor cell.

44. The method of any one of claims 39-43, wherein the extracellular domain comprises a SpyCatcher.

45. The method of any one of claims 39-44, wherein the agent is linked to a SpyTag.

46. The method of any one of claims 39-45, wherein the agent is an antibody, an antibody fragment, a scFv, or a DARPin.

47. The method of any one of claims 39-46, wherein the agent is an antibody and is human IgG.

48. The method of any one of claims 39-47, wherein the cell is a T cell, a Natural Killer (NK) cell, a cytotoxic T lymphocyte (CTL), a macrophage, a stem cell, or a regulatory T cell.

49. The method of any one of claims 39-48, wherein the immune receptor further comprises an intracellular domain of a costimulatory molecule.

50. The method of any one of claims 39-49, wherein the costimulatory molecule is 4-1BB, CD28, CD27, CD2, CD3, OX40, CD30, CD40, PD-1, ICOS, lymphocyte function-associated antigen-1 (LFA-1), CD7, LIGHT, NKG2C, B7-H3, or a ligand that specifically binds with CD83.

51. A method of stimulating a universal immune receptor-mediated immune response to a tumor in a mammal in need thereof, the method comprising
(a) administering to the mammal an effective amount of a cell genetically modified to express an immune receptor comprising a T cell receptor intracellular signaling domain, an intracellular domain of 4-1BB, a transmembrane domain, and an extracellular domain comprising an adaptor molecule, and
(b) administering to the mammal an effective amount of an agent linked to a reciprocal adaptor molecule, wherein the agent specifically binds an antigen expressed by the tumor,
wherein the tumor is pre-determined to express the antigen at an increased level relative to a reference level.

52. A method of treating a cancer in a mammal in need thereof, the method comprising
(a) administering to the mammal an amount of a cell genetically modified to express an immune receptor comprising a T cell receptor intracellular signaling domain, an intracellular domain of 4-1BB, a transmembrane domain, and an extracellular domain comprising an adaptor molecule, and
(b) administering to the mammal an amount of an agent linked to a reciprocal adaptor molecule, wherein the agent specifically binds an antigen expressed by the cancer,
wherein the cancer is pre-determined to express the antigen at an increased level relative to a reference level.

53. The method of claim 51 or 52, wherein the reference level is a level of expression of the antigen on healthy tissue.

54. The method of any one of claims 51-53, wherein the extracellular domain comprises a SpyCatcher or SpyTag.

55. The method of any one of claims 51-54, wherein the agent is linked to a SpyTag or SpyCatcher.

56. The method of any one of claims 51-55, wherein the agent is an antibody, an antibody fragment, a scFv, or a DARPin.

57. The method of any one of claims 51-56, wherein the agent is an antibody and is human IgG.

58. The method of any one of claims 51-57, wherein the SpyTag or SpyCatcher is linked to the agent via light activated site-specific conjugation (LASIC).

59. The method of any one of claims 51-58, wherein the cell is a T cell, a Natural Killer (NK) cell, a cytotoxic T lymphocyte (CTL), a macrophage, a stem cell, or a regulatory T cell.

60. The method of any one of claims 51-59, wherein the cell is an autologous cell.

61. The method of any one of claims 51-60, wherein the cell is contacted with agent prior to administration to the mammal to generate a pre-armed cell and the pre-armed cell is subsequently administered to the mammal.

62. The method of any one of claims 51-61, wherein the cell is administered to the mammal prior to administering the agent to the mammal.

63. A genetically modified cell, a first agent, and a second agent, for use in stimulating a universal immune receptor-mediated immune response to a tumor in a mammal in need thereof, wherein the tumor co-expresses at least two different antigens,
wherein the cell is genetically modified to express an immune receptor comprising a T cell receptor intracellular signaling domain, a transmembrane domain, and an extracellular domain comprising an adaptor molecule,
wherein the first agent is linked to a reciprocal adaptor molecule, and wherein the first agent specifically binds a first antigen expressed by the tumor;
wherein the second agent is linked to a reciprocal adaptor molecule, and wherein the second agent specifically binds a second antigen expressed by the tumor;
wherein the first antigen and the second antigen are different antigens, and wherein the immune receptor, first agent, and second agent are administered to the mammal.

64. A genetically modified cell, a first agent, and a second agent, for use in treating cancer in a mammal in need thereof, wherein the cancer co-expresses at least two different antigens,
wherein the cell is genetically modified to express an immune receptor comprising a T cell receptor intracellular signaling domain, a transmembrane domain, and an extracellular domain comprising an adaptor molecule,
wherein the first agent is linked to a reciprocal adaptor molecule, and wherein the first agent specifically binds a first antigen expressed by the cancer;
wherein the second agent is linked to a reciprocal adaptor molecule, and wherein the second agent specifically binds a second antigen expressed by the cancer; and wherein the first antigen and the second antigen are different antigens.

65. A genetically modified cell and an agent for use in generating a level of lytic activity against a tumor, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds a an antigen expressed by the tumor; wherein an amount of the cell is contacted with an amount of the agent and wherein the amount of the agent relative to the amount of cells is selected to generate the level of lytic activity against the tumor.

66. A genetically modified cell and an agent for use in treating cancer in a mammal in need thereof, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds an antigen expressed by the cancer; wherein an amount of the cell and an amount of the agent is administered to the mammal, wherein the amount of the cell and/or the amount of the agent is selected to provide a level of lytic activity against the cancer.

67. A genetically modified cell and an agent for use in stimulating a universal immune receptor-mediated immune response to a tumor in a mammal in need thereof, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds a an antigen expressed by the tumor; wherein an amount of the cell is administered to the mammal and an amount of the agent is subsequently administered to the mammal.

68. A genetically modified cell and an agent for use in treating cancer in a mammal in need thereof, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds an antigen expressed by the cancer; wherein an amount of the cell is administered to the mammal and an amount of the agent is subsequently administered to the mammal.

69. A genetically modified cell and an agent for use in stimulating a universal immune receptor-mediated immune response to a tumor in a mammal in need thereof,
wherein the cell is genetically modified to express an immune receptor comprising a T cell receptor intracellular signaling domain, an intracellular domain of 4-1BB, a transmembrane domain, and an extracellular domain comprising an adaptor molecule,
wherein the agent is linked to a reciprocal adaptor molecule,
wherein the agent specifically binds a an antigen expressed by the tumor;
wherein an effective amount of the cell is administered to the mammal and an effective amount of the agent is administered to the mammal, and
wherein the tumor is pre-determined to express the antigen at an increased level relative to a reference level.

70. A genetically modified cell and an agent for use in treating a cancer in a mammal in need thereof, wherein the cell is genetically modified to express an immune receptor comprising a T cell receptor intracellular signaling domain, an intracellular domain of 4-1BB, a transmembrane domain, and an extracellular domain comprising an adaptor molecule, wherein the agent is linked to a reciprocal adaptor molecule, and wherein the agent specifically binds a an antigen expressed by the tumor; wherein an effective amount of the cell is administered to the mammal and an effective amount of the agent is administered to the mammal, and wherein the tumor is pre-determined to express the antigen at an increased level relative to a reference level.

71. Use of a genetically modified cell, a first agent, and a second agent, in stimulating a universal immune receptor-mediated immune response to a tumor in a mammal in need thereof, wherein the tumor co-expresses at least two different antigens,
wherein the cell is genetically modified to express an immune receptor comprising a T cell receptor intracellular signaling domain, a transmembrane domain, and an extracellular domain comprising an adaptor molecule,
wherein the first agent is linked to a reciprocal adaptor molecule, and wherein the first agent specifically binds a first antigen expressed by the tumor;
wherein the second agent is linked to a reciprocal adaptor molecule, and wherein the second agent specifically binds a second antigen expressed by the tumor;
wherein the first antigen and the second antigen are different antigens, and wherein the immune receptor, first agent, and second agent are administered to the mammal.

72. Use of a genetically modified cell, a first agent, and a second agent, in the treatment of cancer in a mammal in need thereof, wherein the cancer co-expresses at least two different antigens,
wherein the cell is genetically modified to express an immune receptor comprising a T cell receptor intracellular signaling domain, a transmembrane domain, and an extracellular domain comprising an adaptor molecule,
wherein the first agent is linked to a reciprocal adaptor molecule, and wherein the first agent specifically binds a first antigen expressed by the cancer;
wherein the second agent is linked to a reciprocal adaptor molecule, and wherein the second agent specifically binds a second antigen expressed by the cancer; and wherein the first antigen and the second antigen are different antigens.

73. Use of a genetically modified cell and an agent in generating a level of lytic activity against a tumor, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds a an antigen expressed by the tumor; wherein an amount of the cell is contacted with an amount of the agent and wherein the amount of the agent relative to the amount of cells is selected to generate the level of lytic activity against the tumor.

74. Use of a genetically modified cell and an agent in the treatment of cancer in a mammal in need thereof, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds an antigen expressed by the cancer; wherein an amount of the cell and an amount of the agent is administered to the mammal, wherein the amount of the cell and/or the amount of the agent is selected to provide a level of lytic activity against the cancer.

75. Use of a genetically modified cell and an agent in stimulating universal immune receptor-mediated immune response to a tumor in a mammal in need thereof, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds a an antigen expressed by the tumor; wherein an amount of the cell is administered to the mammal and an amount of the agent is subsequently administered to the mammal.

76. Use of a genetically modified cell and an in the treatment of cancer in a mammal in need thereof, wherein the cell is genetically modified to express an immune receptor comprising a T cell receptor intracellular signaling domain, a transmembrane domain, and an extracellular domain comprising an adaptor molecule, wherein the agent is linked to a reciprocal adaptor molecule, and wherein the agent specifically binds an antigen expressed by the cancer; wherein an amount of the cell is administered to the mammal and an amount of the agent is subsequently administered to the mammal.

77. Use of a genetically modified cell and an agent in stimulating a universal immune receptor-mediated immune response to a tumor in a mammal in need thereof, wherein the cell is genetically modified to express an immune receptor comprising a T cell receptor intracellular signaling domain, an intracellular domain of 4-1BB, a transmembrane domain, and an extracellular domain comprising an adaptor molecule, wherein the agent is linked to a reciprocal adaptor molecule, and wherein the agent specifically binds a an antigen expressed by the tumor; wherein an effective amount of the cell is administered to the mammal and an effective amount of the agent is administered to the mammal, and wherein the tumor is pre-determined to express the antigen at an increased level relative to a reference level.

78. Use of a genetically modified cell and an agent in the treatment of a cancer in a mammal in need thereof, wherein the cell is genetically modified to express an immune receptor comprising a T cell receptor intracellular signaling domain, an intracellular domain of 4-1BB, a transmembrane domain, and an extracellular domain comprising an adaptor molecule, wherein the agent is linked to a reciprocal adaptor molecule, and wherein the agent specifically binds a an antigen expressed by the tumor; wherein an effective amount of the cell is administered to the mammal and an effective amount of the agent is administered to the mammal, and wherein the tumor is pre-determined to express the antigen at an increased level relative to a reference level.
